(19) United States
(12) Patent Application Publication
(10) Pub. No.: US 2022/0101992 A1
(43) Pub. Date: Mar. 31, 2022

(54) SENSOR PATCH AND RELATED SMART DEVICE, SYSTEMS, AND METHODS

(71) Applicants: Alexander D. Porter, Sammamish, WA (US); Daniel J. Daoura, Renton, WA (US)

(72) Inventors: Alexander D. Porter, Sammamish, WA (US); Daniel J. Daoura, Renton, WA (US)

(73) Assignee: PB Inc., Issaquah, WA (US)

(21) Appl. No.: 17/492,657

(22) Filed: Oct. 3, 2021

Related U.S. Application Data
(63) Continuation-in-part of application No. PCT/US2020/026744, filed on Apr. 3, 2021.
(60) Provisional application No. 62/828,943, filed on Apr. 3, 2019.

(51) Int. Cl. G16H 40/63; A61B 90/98; A61B 5/00; A61B 5/024; A61B 5/1455; A61B 5/01; G06K 7/10; G06K 19/07

(52) U.S. Cl. CPC G16H 40/63; A61B 90/98; A61B 5/0022; A61B 5/7465; A61B 5/02438; G06K 7/10297; A61B 5/01; A61B 5/7425; G06K 7/10366; G06K 19/0723; A61B 5/14552

(57) ABSTRACT

In an embodiment, an apparatus, such as a sensor package, includes a sensor, an antenna, and a power circuit. The package is configured for attachment to an object or a subject, for example a patient in need of clinical monitoring. The sensor package is configured to sense a condition or conditions related to the object or subject, and configured to generate a sense signal according to the sensed condition. The sensor patch may include a dual power source and means for switching between power sources, such as NFC and battery. The sensor patch may also include dual radios. As practiced, an NFC radio can be supplemented with a Bluetooth or other LAN radio for data logging. Systems for monitoring sensor patches may include methods for monitoring or tracking location of the patches. In one instance, patches worn by patients are used to triage patients and remotely monitor one or more clinical signs and radio proximity; the system notifies caregivers in real time if the clinical data is indicative of a worsening condition and guides the caregivers to the patient by the shortest possible path, even in a busy facility such as an emergency room.

### CROSS-REFERENCE TO RELATED APPLICATIONS

This application is a Continuation-in-Part of U.S. Application PCT/US20/26744, filed Apr. 3, 2020; which claims priority to U.S. Provisional Patent Application No. 62/828,943, filed on Apr. 3, 2019, the contents of which are incorporated herein in their entirety. This application is further related to U.S. patent application Ser. No. 17/163,403, filed Jan. 30, 2021, which is incorporated herein in full by reference.

### SUMMARY

Embodiments of the disclosure relate to an affixable sensor for sensing, determining, monitoring, and reporting of clinical conditions or physical qualities.

The internet of things (IoT) offers a world linked by a highly granular sensor net. It is anticipated that maturation of higher-density information networks will rely on increased miniaturization of wireless sensors (e.g., temperature sensors, humidity sensors, accelerometers, and transducers) for conveying relevant information, in analog or digital form, to user devices, remote workstations, or cloud-based computing machines. It is anticipated that this evolution will be achieved by the convergence of improvements in silicon-based manufacturing, ink printed circuitry, wearable electronics, and radio technologies. An elusive goal has been the capacity of sensors, devices, and systems to operate cordlessly and without battery power. The use of a battery as a substitute for a power cord may not be an optimal solution because a battery occupies space on a hardware device and may have a relatively short lifetime. Thus, expansion of the IoT into daily life has been slowed by the lack of a solution for wirelessly delivering power to a battery-less device (e.g., a sensor) for sensing, monitoring, determining, and reporting one or more physical quantities (e.g., temperature) or one or more conditions (e.g., environmental).

The capacity to power a device, such as a sensor device, passively, via energy harvested wirelessly by the sensor device from near-field emissions, offers a needed solution. Near-field wireless power transfer refers to energy transfer inside a working distance, which may be defined by, or otherwise related to, a wavelength or frequency at which a transmitter transmits a signal from which the sensor device derives power. For example, proximate application of an electromagnetic field from a portable device, such as a smart device (e.g., a smart phone), can be sufficient to power a sensor device located within an approximate range of 0-24 inches of the portable device. Further in example, a sensor device enabled to harvest power from an electromagnetic field is configured to sense, to monitor, and to report sensor quantities and conditions. The reporting may include both wireless “notification” to a proximate smart device and via a non-telemetric reporting means such as a visual display (e.g., one or more light-emitting diodes (LEDs)), acoustic signal, or haptic signal generated by the sensor device so as to draw the attention of a human user and to communicate the sensor output (e.g., temperature value or range) directly to the user. Temperature may be reported, for example, both telemetrically and visually.

Disclosed are one or more embodiments of a NFC (“near-field communication”) sensor-patch device, or sensor patch) for sensing, detecting, measuring, or monitoring a condition of, or a physical quantity related to, an object or a living being. Examples of such a condition including an environmental condition such as a level of ambient light and a level of noise and a living-body condition such as heart rate or respiratory rate. Examples of such a physical quantity include temperature, humidity, linear velocity, angular velocity, altitude, and pressure. Such a sensor patch can be configured to report sensor data wirelessly via an NFC data channel to a smart device such as a smart phone. The sensor patch also can include a “reporter” component (e.g., one or more LEDs) configured to report sensor data directly, in real time, to a human user. The NFC signal over which the smart device and the sensor patch wirelessly exchange data also powers the reporter component and the other components and circuits of the sensor patch The sensor patch's local reporter display can be complementary to a wireless notification of the sensor output made to a proximate smart device, such as a smartphone that generates an NFC signal from which the sensor patch draws power.

In other examples, conditions/quantities that such a sensor patch may sense include temperature, pressure, vibration, moisture, humidity, pH, glucose, chemical composition associated with a living body, a nonliving object, or an environmental state, movement such as acceleration, velocity (linear and angular), and position.

The sensor patch uses the harvested power from an RF field to make one or more sensor measurements, and to report the result(s) locally, to a remote receiver, or both locally and to a remote receiver. Such a sensor patch does not require power from a battery and can be powered at the point of care only when a sensor measurement is desired. The sensor patch can include one or more antennas configured to receive a power-and-data signal from which the sensor device is configured to derive power, and the sensor device can be configured to receive data from a remote device by demodulating the power-and-data signal and can be configured to transmit data to the remote device by modulating the power-and-data signal. The sensor patch can be configured to perform two or more of the power deriving, data receiving, and data transmitting simultaneously. Further, the process of powering the sensor patch and measuring one or more quantities or conditions with the powered sensor patch can be repeated an indefinite number of times. And a remote device, such as a smart device, can be programmed to perform one or more kinds of sensor measurements without the need for specialized instruments dedicated to each kind of sensor, eliminating redundancy by providing a multifunctional sensor patch or a family of sensor patch types all operable from a single smart device.

Sensor data may be useful both at a point of measurement and at a remote host. For example, a sensor patch can be configured to transmit temperature sensor data to a remote workstation for charting, archiving, or making remote notifications (such as for cold-chain verification, fault detection, and temperature monitoring of a living being), but also for local use such as for providing an immediate indication of the result to, for example, a caretaker of a person whose body temperature the sensor patch is configured to sense. As configured for local use in measuring temperature, for example, a visual indicator is integrated into the sensor patch and is configured to display the temperature result parametrically (e.g., a numerical display) or non-parametrically (e.g., a color) to a user.

A sensor patch, according to an embodiment, is remotely powered by electromagnetic (EM) near-field emission from a device, such as a smart device, and a user, other human, or imaging machine can read, directly, a visual reporter display of the sensor patch. As used herein, a “reporter” is a local electronic display or other indicator of the sensor patch, and a “notifier” includes a radio signal that the patch sensor transmits to a user's device (e.g., a smart phone), another remote device, an administrator, or a host.

In an embodiment, a sensor patch has a unique identifier that the sensor patch stores electronically. Data from the sensor can be tagged with the sensor identifier before reporting it to a smart device (e.g., a smart phone) over an NFC carrier signal, and the smart device can be operated to transmit, or otherwise to forward, the data as digitized information representing physical measurements taken by the sensor patch. The transmission can be, for example, via a Bluetooth radioset (e.g., radio or radio circuit), a Wi-Fi radioset, a cellular radioset, an NFC radioset, or over any other frequency band and field. The transmission also can be by wired means. One also can use the sensor patch for temperature monitoring of a product (e.g., a cold-chain application) and for clinical measurements.

In an embodiment, a sensor patch can be configured to sense, to determine, and to provide a clinical temperature. The sensor patch is adhered to skin so as to operate without dermal penetration and is capable of being held in place by an adhesive. The sensor patch can be more comfortable than an invasive device such as an insertable ear, rectal, and sublingual thermometer, or an implantable sensor capsule, all of which require that the measuring device be placed inside an orifice or tissue of the body. In contrast, a conventional insertable thermometer generally has a disposable probe cover so that bodily fluids are not transmitted from one patient to the next, and take significant time (e.g., fifteen seconds to one minute) to use per patient due to the need to install and dispose of the cover and to allow sufficient time for the thermometer reading to settle to a measured value. And the stocking probe covers for a conventional thermometer can require significant storage space, can be costly, and, therefore, can be a nuisance. But with an embodiment of the sensor patch, the sensor element (e.g., temperature sensor) being separate from the read device (e.g., a smart phone) can increase efficiency by reducing use time (e.g., the temperature sensor, once up to temperature, stays up to temperature as long as the sensor patch is affixed to the person or object whose temperature is being measured) and can reduce the instance of cross-user contamination and infection.

In addition, a remote device, such as a smart device, used in combination with a sensor patch, can be configured to send, to the sensor patch, configuration information or program instructions for carrying out one or more sensor measurements simultaneously or in sequence, and for displaying the results to the person whose temperature is being measured or to a caregiver of such person. Measurement data may be stored in memory of the smart device (e.g., smart phone) or of a remote workstation for later retrieval or for real-time plotting to show trends and correlations with earlier results. The sensor patch can be configured to send a measurement requiring attention (e.g., a temperature measurement indicative of a fever) as a notification to a caregiver (e.g., the parent of a sick child) who may or may not be in proximity to the sensor patch. For example, the sensor patch may send such a notification to the smartphone of a caregiver.

Another conventional battery-powered thermometer device is configured for use in the following manner. One contacts the forehead of a subject with the device and then draws the device across the skin of the forehead. The battery-powered thermometer device provides a readout of a value that indicates the presence or absence of a febrile condition or hypothermia. But the device cannot operate passively (e.g., without a battery), and because the device relies on rate of change of heat transfer rather than equilibrium temperature, the device can be less accurate than other types of conventional thermometers, for example, when measuring the temperature of a human body that is in a cold sweat.

In some instances, the sensor patch includes a thermistor for measuring temperature, an RF antenna coil for harvesting energy and for communicating data across an NFC radioset, and the thermistor is disposed on a probe outside the bounds of the antenna coil so that it can be inserted into a cavity in a body while still enabling the antenna to be positioned closed to a smart device that supplies the needed RF excitation energy to power the sensor. In one embodiment, there is also an LED positioned within the bounds of the antenna coil so that all heat generating elements are isolated from the thermistor by a low-thermal conductivity substrate. The LED may be an RGB-LED.

A sensor for pressure also may be of interest clinically, and in a sensor-patch embodiment having a multi-sensor element, a pressure sensor is provided in addition to, or in place of, a temperature sensor.

For example, pressure within one's radial artery and pulse rate are of interest in combination with temperature for determining clinical states associated with endotoxic shock, anaphylactic shock, and hypothermia. Radial blood pressure is also of interest as a measurement of pulse for improving athletic performance and for cardiac rehabilitation, for example.

Sensor patches may be worn and readily checked on the go, so that early detection of fever, as for coronavirus surveillance, is readily achieved without the need to carry anything more than a smart device. The sensor devices also may be operated as part of a system for delivering telemedicine to remote rural locations, and, therefore, an embodiment of a sensor patch can reduce the need for a subject's hospitalization, emergency-room treatment, and travel for physician-office visits.

The sensor patch uses the harvested power to make one or more sensor measurements, and to report the result(s) locally, to a remote receiver, or both locally and to a remote receiver. Such a sensor patch does not require power from a battery and can be powered at the point of care only when a sensor measurement is desired. The sensor patch can include one or more antennas configured to receive a power-and-data signal from which the sensor device is configured to derive power, and the sensor device can be configured to receive data from a remote device by demodulating the power-and-data signal and can be configured to transmit data to the remote device by modulating the power-and-data signal. The sensor patch can be configured to perform two or more of the power deriving, data receiving, and data transmitting simultaneously. Further, the process of powering the sensor patch and measuring one or more quantities or conditions with the powered sensor patch can be repeated an indefinite number of times. And a remote device, such as a smart device, can be programmed to perform one or more kinds of sensor measurements without the need for specialized instruments dedicated to each kind of sensor, eliminating redundancy by providing a multifunctional sensor patch or a family of sensor patch types all operable from a single smart device

In an embodiment, the sensor patches are disposable and inexpensive. The remote devices, such as smart devices, used for powering a sensor patch and for receiving sensor output may not be single-purpose units, but instead can be configured to interact with various classes of sensor patches and sensors on those sensor patches.

In an embodiment, a sensor device, such as a sensor patch, includes multiple sensor elements and an operator need only select the kind of measurement to be made and the smart device is programmed to do the rest, including configuring the sensor device to make the selected measurement(s).

In contrast, current practice typically requires an armamentarium of devices to collect the range of data commonly needed in clinical monitoring of a subject. But one or more sensor patches, according to an embodiment, can slash the required specialized equipment needed to do clinical monitoring of a subject's temperature and other vital signs and makes a commonly available smart device the center of an “ecosystem” of disposable sensor devices (e.g., sensor patches) that require no specialized training to use and are not dedicated to any particular kind of testing. Thus, an embodiment of an NFC-driven system disclosed herein can have a dramatic effect in reducing the cost of delivering medical care and can enable sensor data to be digitized and shared to any remote monitoring site such as a nursing station, or across a continent, or across the world. Once digitized, the data can be stored in a database for later retrieval, and by assigning each sensor patch a unique digital identifier, data automatically is associated with the correct user or subject profile. For example, an RFID subject armband or QR code can be used to associate the sensor patch with the correct subject, and once this is done, the sensor patch is programmed to deliver data to the correct subject file. Such a sensor patch also may be used for home healthcare as an inexpensive disposable “bandage” type sensor patch that is configured to provide sufficient information to monitor a sick child, for example, via the instantaneous local display of temperature or other vital sign(s).

An embodiment of a sensor patch also can be adhered to inanimate objects such as a wine bottle or other container holding perishables where, for example, temperature of the container contents is critical. By supplying each sensor patch with electronic memory, the sensor patch can be configured to store a temperature history of the object (e.g., one temperature per NFC access/scan) and upload the stored temperature history of the object from the sensor-patch memory to a smart device simply by placing the sensor patch within NFC proximity of the smart device and by activating, with a configurable application, the smart device to read and to report the current temperature and temperature history that the sensor patch senses and provides.

In an embodiment, each sensor patch has a unique identifier that the sensor patch is configured to send digitally to a corresponding smart device when the sensor patch makes a measurement, and the user has an option to associate that digital identifier with a particular subject (e.g., patient, child) or inanimate object about which the sensor patch is collecting sensor data. An application program installed on the smart device can provide user profiles to be attached to particular subjects or objects so that the smart device, in response to the application program, can aggregate sensor data over time, and can plot or track trends in the sensor data

For some sensors, power from the NFC/RFID antenna, via the rectifier (e.g., half-wave or full-wave), is sufficient to execute a sensor measurement and make a report that includes a sensor readout. But more generally, for some embodiments, a circuit LDO switching regulator may enable an NFC/RFID transceiver to be placed in proximity to the device so as to initialize a battery power supply to the microcontroller and to establish identifiers, pairing, and other conditions, including private keys, for secure exchange of data. During periods of non-use, the switching regulator may disconnect the battery, so that battery discharge during quiescent periods is limited to self-discharge as dependent on internal battery chemistry alone. Interestingly, a body temperature sensor reporting ambient temperature, as would be consistent with a sensor patch that has been detached from a subject, can trigger a battery disconnect so as to realize highly efficient control of battery shelf life. These features can be accompanied by notifications to the user, and can be monitored by a cloud administrator so that data can be restored if still needed.

### BRIEF DESCRIPTION OF THE DRAWINGS

These and other features of sensor devices, such as sensor patches, and related smart devices, software application, configuration data, systems, and methods are within the scope of the disclosure. The elements, features, steps, and advantages of embodiments of the patches, devices, software applications, configuration data, methods, and systems will be more readily understood upon consideration of the following detailed description, taken in conjunction with the accompanying drawings, in which embodiments are illustrated by way of example.

It is to be expressly understood, however, that the drawings are for illustration and description only and are not intended as a definition of the scope and limits of the disclosed devices, software applications, configuration data, systems, and methods. The various elements, features, steps, and combinations thereof that characterize embodiments of the subject matter disclosed herein are pointed out with particularity in the claims annexed to, and forming part of, this disclosure.

The teachings of the present disclosure are more readily understood by considering the drawings, in which:

FIG. 1 is a circuit block diagram of an NFC-powered sensor patch configured for deriving operating power from an NFC signal generated by a proximate NFC-capable smart device, for reporting one or more sensor conditions in a local display mode, and for exchanging data with a proximate NFC-enabled smart device by modulating and demodulating the NFC signal, according to an embodiment.

FIG. 2A is a schematic diagram of a temperature sensor suitable for use in the sensor patch of FIG. 1, according to an embodiment.

FIG. 2B is a schematic diagram of a pressure sensor suitable for use in the sensor patch of FIG. 1, according to an embodiment.

FIG. 2C is a schematic diagram of a red-green-blue (RGB) light-emitting-diode (LED) array suitable for use as a sensed-value reporter in the sensor patch of FIG. 1, according to an embodiment.

FIG. 3 is a diagram of a sensor patch including an antenna, a controller circuit, a communication circuit, one or more sensors, and one or more electronic reporter components, according to an embodiment.

FIG. 4A is a schematic diagram of the circuitry of the sensor patch of FIG. 1 according to an embodiment, the circuitry including a controller and a temperature sensor with a local reporter display (here an RGB LED array).

FIG. 4B is a schematic diagram of a circuitry of the sensor patch of FIG. 1, according to another embodiment, the circuitry including a controller circuit, a temperature sensor, and a pressure sensor.

FIG. 5A is a plan view of a sensor patch measuring less than 2 centimeters (cm) in diameter and less than 1 millimeter (mm) in thickness, according to an embodiment. FIG. 5B is a cross-section view of the device taken at section A-A to show a printed bridge over the antenna coils.

FIG. 6 is a circuit diagram of a sensor patch having multiple banks of sensors and a battery configured to store energy to power the sensor patch during extended measurement cycles, according to an embodiment.

FIG. 7A is an exploded view of a multi-layer adhesive sensor patch that can include the circuitry of one or more of FIGS. 1-6, and that includes a visual indicator (LED), a microcontroller, and a thermistor (temperature-sensing element) optionally integrated within the microcontroller, according to an embodiment.

FIG. 7B is an exploded cross-sectional view, along a midline, of the multi-layer adhesive sensor patch of FIG. 7A, according to an embodiment.

FIG. 8 is a plan view of a sensor patch with alternate form factor.

FIG. 9 is a diagram of a system configured for making sensor measurements and for networking sensor data to a local-area network (LAN) or a wide-area network (WAN) via Wi-Fi, to one or more cellular networks, or to a combination or sub-combination of any of the aforementioned networks and other networks, according to an embodiment.

FIG. 10 is a flow diagram of a method for making a sensor measurement on a human body, for example, with a sensor patch of one or more FIGS. 1 through 8, according to an embodiment.

FIG. 11 is a diagram of a caregiver taking a temperature of a subject using the sensor patch of FIGS. 1 through 8 and a smartphone running a sensor-patch software application, according to an embodiment.

FIG. 12 is a circuit diagram of a sensor patch configured for deriving operating power from an NFC signal generated by a proximate NFC smart device, for reporting one or more sensed quantities and conditions in a local display mode, and for exchanging information with a proximate NFC smart device by modulating and demodulating the NFC signal, according to an embodiment.

FIG. 13 is an exploded view of a five-layer adhesive sensor patch that can include the circuit of FIG. 12, according to an embodiment.

FIG. 14 is a plan view of a smartphone displaying, on a display screen, a user interface of a sensor-patch software application that configures the smartphone for use with the sensor patch of FIGS. 12-13, according to an embodiment.

FIG. 15 is a flow diagram of a method for downloading, installing, and setting up a sensor-patch software application for use with the sensor patch of FIGS. 12-13, according to an embodiment.

FIG. 16 is a flow diagram of a method for measuring a condition, such as temperature, of an object, such as the child of FIG. 11, using a smart device and the sensor patch of FIGS. 12-13, according to an embodiment.

FIG. 17A is a schematic of a circuit for measuring corrected body temperature using a pair of thermometric sensors separated by a thermally conductive mass of known heat conductance.

FIGS. 17B and 17C are perspective drawings of a plug-in thermistor for use in a smartphone to measure ambient temperature.

FIG. 18 is a circuit diagram of a smart device, such as the smartphone of FIG. 11, that can be configured for use with a sensor patch, such as one or more of the sensor patches of FIGS. 1-8 and 12-13, according to an embodiment.

FIGS. 19A, 19B, 19C, 19D, 19E, 19F, 19G, and 19H are views of screenshots on a graphical user interface for operation of a method of measuring a temperature according to software installed as described in FIG. 15.

FIG. 20 is a schematic of a dual-powered sensor patch device.

FIG. 21 is a system schematic of an IOT network with sensor patch, hub, personal smart device, and cloud host forming a cellular/Bluetooth global area network as used in patient clinical monitoring and related services.

FIG. 22 is a schematic of a dual-powered, dual-radio sensor patch device.

FIG. 23 illustrates a global area network combining a dual-radio dual-powered sensor patch with cellular link to a cloud host.

FIG. 24 is a schematic of an alternative dual-powered, dual-radio sensor patch device functional as a data logger.

FIG. 25 is a schematic of an integrated dual-powered, dual-radio sensor patch device functional as a data logger.

FIGS. 26A and 26B show a plan and section schematic view of a dual radio, dual power sensor patch with two-piece assembly details.

FIGS. 27A and 27B are front and back views of a sublingual thermometer having circuitry of the preceding drawings.

FIGS. 28A and 28B are exploded views of a clamshell construction of the sublingual thermometer of the preceding figure.

FIGS. 29A, 29B, 29C, 29D, 29E and 29F are design views of the sublingual thermometer of FIGS. 27A through 28B.

FIG. 30 is a printed circuit schematic of a sensor patch for topical use.

FIGS. 31A, 31B, 31C, 31D, 31E and 31F are design views of sensor patches with NFC radio circuitry.

FIG. 32 illustrates a sensor patch used in the context of a gate or portal for screening entrants to a facility.

FIGS. 33A and 33B are circuit diagram schematics of a sensor patch with battery circuit and clip on bridge, according to another embodiment.

illustrates a sensor patch used in the context of a gate or portal for screening entrants to a facility.

FIG. 34 is a schematic view of a patch with thermistor disposed outside the NFC coil.

FIG. 35 illustrates an alternate sensor patch for use with an NFC/RFID gateway or portal.

FIGS. 36A and 36B are circuit diagrams of a band-shaped sensor patch powered by NFC.

FIG. 37 is a view of a circuit 3900 with switching regulator for dual power supply to a processor and sensor, the processor having an NFC/RFID radioset and communications circuit and a Bluetooth radiocircuit in the processor core. The circuit includes flash memory and proprietary encryption and security features for protecting data.

The drawings are not necessarily to scale. Certain features or components herein may be shown in somewhat schematic form and some details of conventional elements may not be shown in the interest of clarity, explanation, and conciseness.

### Glossary

Certain terms are used throughout the following description to refer to particular features, steps or components, and are used as terms of description and not of limitation. As one skilled in the art will appreciate, different persons may refer to the same feature, step, or component by different names. Components, steps, or features that differ in name but not in structure, function, or action are considered equivalent, and may be substituted herein without departure from the scope of the disclosure. Certain meanings may be defined here as intended by the inventors, i.e., they are intrinsic meanings. Other words and phrases used herein may take their meaning as consistent with usage as would be apparent to one skilled in the relevant arts. One can interpret the meaning of a term listed in the Glossary based on the Glossary description of the term, on the use of the term elsewhere in this disclosure, or on a combination of the Glossary description of the term and the use of the term elsewhere in this disclosure.

Unless otherwise described herein, all technical and scientific terms used herein have the same meaning as commonly understood by one of ordinary skill in the art to which this subject matter belongs.

A “sensor device” can be a device or apparatus configured for making, collecting, and reporting one or more measurements of a condition (e.g., fever) or a physical quantity (e.g., temperature) while attached (e.g., with an adhesive) to a living being (e.g., a human) or to an inanimate object (e.g., a bottle of wine, a food-serving platter). A “sensor patch” can be a type or category of a “sensor device.”

“Exemplary” and “embodiment” can mean “serving as an example, instance, configuration, version, implementation, or illustration.” Unless stated otherwise herein, no embodiment or example is considered to be preferred over any other embodiment.

“Wireless power” can mean any form of energy associated with electric fields, magnetic fields, electromagnetic fields, electric signals, electromagnetic signals, or otherwise that is transmitted from a transmitter to a receiver without the use of physical electromagnetic conductors. For example, the following non-limiting list of devices can be powered, charged, or recharged wirelessly: mobile phones, cordless phones, iPods, MP3 players, Bluetooth® and other wireless headsets. A type of wireless energy transfer includes magnetic coupling, such as magnetic-coupled resonance, using frequencies, for example, below 30 MHz. But various frequencies may be employed including frequencies where license-exempt operation at relatively high radiation levels is permitted, for example, at either below 135 kHz or at 13.56 MHz. At these frequencies, which are normally used by Radio Frequency Identification (RFID) systems, systems typically comply with interference and safety standards such as EN 300330 in Europe or FCC Part 15 norm in the United States.

“Smart device” can mean any of the class of devices (e.g., a smart phone, a tablet computer) that derived from pagers and cellphones and are miniature computers with radiosets capable of addressing, for example, local-area networks and broad-area networks. These devices are typically programmable (e.g., by downloading and installing a software application often called an “app”) or are otherwise software, firmware, or data-stream configurable for a wide variety of uses. Individual device permissions can limit access, and specialized encoding may be applied to data to prevent unauthorized parties from recovering that data. Smart devices are well known in a variety of communications technologies, including Wi-Fi®, direct Wi-Fi®, Bluetooth®, and NFC/RFID protocols, but also have antennas that may be tuned to resonate, or otherwise to operate, at frequencies compatible with powering contactless sensor elements. A smart device may be configured, by an “app,” to receive or to transfer power wirelessly according to, for example, an NFC or RFID protocol. Furthermore, “smart devices” that are Wi-Fi®, direct Wi-Fi®, or Bluetooth® enabled may also be NFC or RFID enabled, and, therefore, may include one or more NFC or RFID antennas that may be configured to emit radio energy at frequencies compatible with powering contactless sensor devices such as a sensor patch. An “app” installed on a smart device may configure, or otherwise enable, the smart device to discharge radio energy according to an NFC protocol to power a battery-less temperature sensor device such as a temperature sensor patch.

“Passive NFC mode” can mean an operational mode in which only one or more, but not all, NFC devices in a system are active in that it/they each generate a respective power signal. The remaining one or more devices are passive in that it/they may receive, but do not generate, a power signal. In a “passive NFC mode,” any NFC device in the system can receive and transfer data via a power signal by demodulating and modulating, respectively, the power signal.

“Active power mode” relates to an operational mode in which the devices in a system are autonomously powered, and are capable of generating a radio signal using on-board power. In some instances, devices can toggle between passive NFC and active power modes.

“Computer” can mean a virtual or physical computing machine that includes computing circuitry, that accepts information in analog or digital form, and that manipulates the information (typically after conversion into digital form if not already in digital form) for a specific result based on a sequence of program instructions or based on circuitry (e.g., a field-programmable gate array (FPGA)) that is configured to implement an algorithm. Examples of a “computer” include a desktop computer, a laptop computer, a tablet computer, a smart device such as a smart phone, and a server computer.

“Computing machine” can mean an electronic apparatus that includes logic circuitry having one or more processor circuits or controller or control circuits (e.g., a microprocessor or microcontroller), programmable memory or firmware-configurable circuitry (e.g., an FPGA), volatile memory, non-volatile memory, and one or more ports to I/O devices such as a pointer, a keypad, a sensor, imaging circuitry, a radio or wired communications link, and so forth. A computing machine can be networked with other computing machines via conventional wireless or wired connections. Controllers are generally supported by volatile and non-volatile memory, a timing clock or clocks, and digital input and output circuits, as well as one or more communications protocols. Furthermore, computers are frequently formed into networks, and a network of computers may be referred to by the term “computing machine.” In one instance, one or more computing machines “in the cloud” can form a “cloud” computing machine.

“Server” can mean a computing machine which executes software and which provides one or more services to a virtual client (e.g., a software program running on the server) or to an actual client (e.g., another computer or computing machine directly connected to the server, or connected to the server via a network such as the internet). A client typically has a user interface and performs some or all of the processing on data or files received from the server, but the server typically maintains the data and files and processes the data requests. A “client-server model” divides processing between one or more clients and one or more servers, and refers to an architecture of the system that can be co-localized on a single computing machine or can be distributed throughout a network or the “cloud.”

“Processor” can mean a digital device, such as a digital integrated circuit (e.g., a microprocessor or microcontroller) that processes information in digital form and manipulates the information for a specific result based on a sequence of programmed instructions, or can mean a hardwired pipeline that can be configured with a stream of configuration values. Processors are used as parts of digital circuits generally including a clock, volatile memory and non-volatile memory (e.g., containing programming instructions), and may interface with other digital devices or with analog devices through Input/Output (I/O) ports, for example. Other names for “processor” include “controller,” “control circuit,” “controller circuit,” “microprocessor,” and “microcontroller.”

General connection terms including, but not limited to “connected,” “attached,” “conjoined,” “secured,” “coupled,” and “affixed” are not meant to be limiting, such that structures so “associated” may have more than one way of being associated. For example, there may be zero, one, or more than one component disposed (e.g., in series) between two components that are described as being “coupled” or “connected” to one another. “Fluidly connected” indicates a connection for conveying a fluid therethrough. “Digitally connected” indicates a connection in which digital data may be conveyed therethrough. “Electrically connected” indicates a connection in which units of electrical charge are conveyed therethrough.

Relative terms should be construed as such. For example, “front” can be relative to “back,” “upper” can be relative to “lower,” “vertical” can be relative to “horizontal,” “top” can be relative to “bottom,” “inside” can be relative to “outside,” and so forth. Unless specifically stated otherwise, ordinals such as “first,” “second,” “third,” and “fourth” can be for purposes of designation and not for order or for limitation. Reference to “one embodiment,” “an embodiment,” or an “aspect,” means that a particular feature, structure, step, combination or characteristic described in connection with the embodiment or aspect is included in at least one realization of the present teachings. Thus, the appearances of the phrases “in one embodiment” or “in an embodiment” in various places throughout this specification are not necessarily all referring to the same embodiment and may apply to multiple embodiments. Furthermore, particular features, structures, or characteristics of two or more of the embodiments may be combined in any suitable manner in one or more other embodiments, even if such a combination or other embodiment is not expressly described herein.

Claims not including a specific limitation should not be construed to include that limitation. For example, “a” or “an” as used in the claims does not exclude a plurality.

“Conventional” refers to a term or method designating that which is known and commonly understood in the technology to which these teachings relate at the time of the earliest priority date of the patent application.

Unless the context requires otherwise, throughout the specification and claims that follow, “comprise” and variations thereof, such as, “comprises” and “comprising,” and other open-ended terms such as “including,” are to be construed in an open, inclusive sense—as in “including, but not limited to.”

A “method” as disclosed herein refers to one or more steps or actions for achieving the described end. Unless a specific order of steps or actions is required for proper operation of the embodiment, the order and/or use of specific steps and/or actions may be modified without departing from the scope of the present disclosure. And the order of steps or actions in a method claim does not limit the method claim to that order unless expressly stated or unless the method is inoperable if the steps are performed in any other order.

“Approximately,” “substantially,” and similar words, as used herein, indicate that a given quantity b can be within a range b±10% of b, or b±1 if |10% of b|<1. “Approximately,” “substantially,” and similar words, as used herein, also indicate that a range |b−c| can be from |b−0.10|(c−b)| to c+0.10|(c−b)∥. Regarding the planarity of a surface or other region, “approximately,” “substantially,” and similar words, as used herein, indicate that a difference in thickness between a highest point and a lowest point of the surface/region does not exceed 0.20 millimeters (mm).

### DETAILED DESCRIPTION

The detailed description set forth below in connection with the appended drawings is intended as a description of exemplary embodiments and is not intended to represent the only embodiments in which the present subject matter can be practiced. The term “exemplary” used throughout this description means “serving as an example, instance, or illustration,” and should not necessarily be construed as preferred or advantageous over other exemplary embodiments. The detailed description includes specific details for the purpose of providing a thorough understanding of exemplary embodiments of the disclosure but may include other details for purposes of clarity. It will be apparent to those skilled in the art that at least in some instances, embodiments may be practiced without these specific details. And in some instances, well-known structures and devices are shown in block-diagram form in order to avoid obscuring the novelty of the exemplary embodiments presented herein.

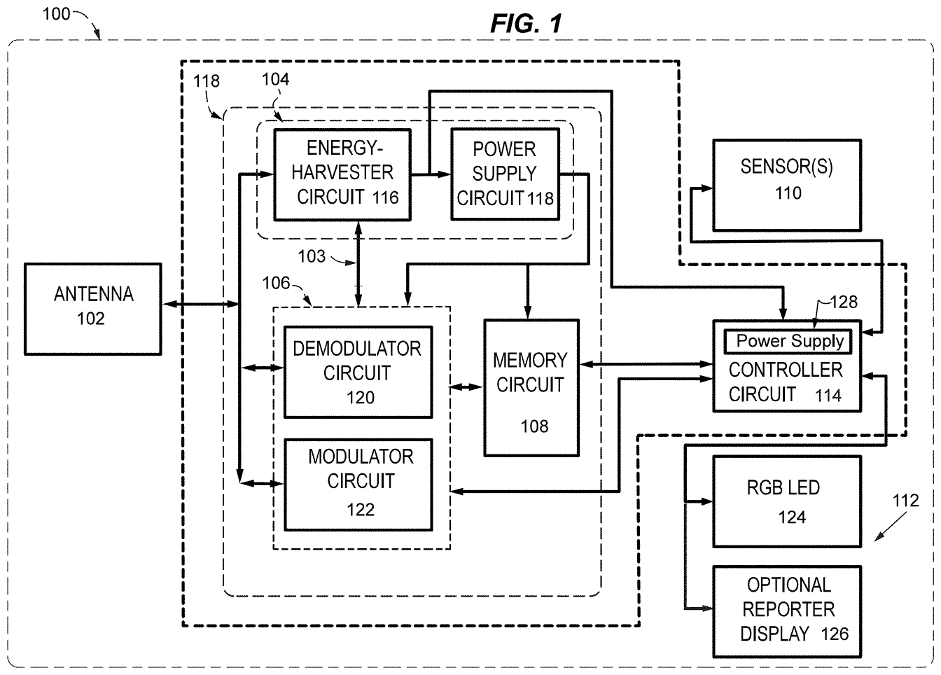

FIG. 1 is a circuit diagram of a sensor device, here an NFC-powered sensor patch 100, configured for passive operation, for reporting one or more sensor quantities or conditions by a local display mode, and for data exchange with a proximate smart device by demodulating and modulating an NFC field or signal, according to an embodiment.

NFC (near-field communication) is a technology for exchanging information (e.g., data) and transferring power between two devices via an electromagnetic field or signal, hereinafter an “NFC signal” or “signal.” An example of an NFC system is a smart card and a smart-card reader. The smart-card reader is configured to transmit an NFC signal, to modulate the NFC signal with another signal that represents data or one or more commands, and to demodulate the NFC signal to recover data or one or more commands that the smart card transmits. The smart card is configured to derive operational power from the NFC signal, to demodulate the NFC signal to recover data and commands transmitted by the smart-card reader, and to modulate the NFC signal with another signal that represents data or one or more commands. In an embodiment, the smart-card reader and the smart card can be configured to modulate and demodulate the NFC signal at respective times (e.g., time-division multiplexing) or simultaneously (e.g., frequency-division multiplexing). The antennas of the reader and the smart card can be configured to resonate at the frequency of the NFC signal to facilitate power transfer. The resonant frequency of the reader and card antennas, while coupled in the near field, can be related to the respective inductance and capacitance of each antenna and the respective conductance and capacitance that each of the antennas presents to the other antenna.

Embodiments include a device to couple power to another device via the device antennas, which are in the near fields of each other.

A smart device (not shown in FIG. 1) includes dual functionality for emitting wireless power on a carrier wave via an NFC wireless power transmitter and for engaging in bidirectional NFC with the sensor patch 100. The smart device includes an NFC transceiver configured to emit an active electromagnetic or magnetic field and to transmit and receive data to and from the NFC passive sensor patch 100 while the smart device is in proximity to (e.g., within approximately 0-12 inches of) the NFC antenna 102.

In addition to the antenna 102, the patch sensor 100 includes a power circuit 104, a communication circuit 106, a memory circuit 108, a sensor circuit 110, an indicator 112, and a controller circuit 114.

The antenna 102 can be any type of antenna that is suitable for NFC, and that is suitable to generate, from an NFC source signal received from a remote device (e.g., a smart phone), a receive signal of a strength sufficient to power the patch sensor 100 (e.g., typically, the larger the cross-sectional area of the antenna, the more power the energy-harvester circuit 116 can extract from the NFC source signal) with a sufficiently high supply voltage (e.g., typically the more turns/windings/loops that the antenna has, the higher the voltage of the receive signal that the antenna can generate across its nodes). Furthermore, the antenna 102 can be a group of multiple antennas that cooperate to receive an NFC source signal and to generate a receive signal in response to the source signal. Moreover, in an embodiment where the antenna 102 is an inductive antenna, the circuit nodes, e.g., of the energy-harvester circuit 116, of the communications circuit 106, or of any other circuitry to which the antenna is coupled, present, to the antenna, a capacitance having a value that effectively forms, with the inductive antenna, a parallel LC, or tank, circuit having a resonant frequency

$$\frac{1}{2 π \sqrt{LC}} ∼ 13.56 megahertz ( MHz ) ,$$

where L is the inductance of the antenna, and C is the capacitance across the circuit nodes. The circuitry coupled to these circuit nodes typically is, or includes, an impedance-matching network that, at least ideally, allows maximum, or near-maximum, power transfer from the antenna 102 to the power circuit 104 and the communications circuit 106. For example, such an impedance-matching network may be disposed between the antenna and the power and communication circuits 104 and 106.

The power circuit 104 is configured to harvest power from the NFC receive signal generated by the antenna 102, to provide the harvested power to other circuits and components of the sensor patch 100, and includes an energy-harvester circuit 116 and a power-supply circuit 118. The energy-harvester circuit 116 is configured to convert the receive signal from the antenna 102 into a raw power signal. For example, the energy-harvester circuit 116 includes a conventional half-wave or full-wave rectifier (not shown in FIG. 1) that is configured to generate a rectified signal, and one or more low-pass filters (not shown in FIG. 1) configured to generate the raw power signal by reducing the ripple superimposed on the rectified signal. And the power-supply circuit 118 is configured to convert the raw power signal into a regulated power signal having a regulated voltage, for example, in an approximate range of 1.8 Volts (V) to 2.5 V. For example, the power-supply circuit 118 can be any suitable type of voltage regulator, such as a linear regulator, a buck converter, a boost converter, a buck-boost converter, or a flyback converter.

The communication circuit 106 is configured to recover source information from the NFC source (e.g., a smart phone, not shown in FIG. 1) that generates the NFC source signal, is configured to transfer sensor-patch information from the sensor patch 100 to the source, and includes a demodulator circuit 120 and a modulator circuit 122. The NFC source is configured to transmit source information, such as source commands or other source data, to the sensor patch 100 by modulating a carrier signal with the source information to generate the source signal; that is, the source signal is configured to provide power, and to carry source information, to the sensor patch 100. For example, the source is configured to generate the source signal by amplitude, frequency, or phase modulating a sinusoidal carrier signal with a source information signal that represents the source information; further in example, the source is configured to generate the source signal by amplitude-shift-key (ASK) modulating a sinusoidal carrier signal with a source information signal that represents the source information. The demodulator circuit 120 is configured to recover the source information from the receive signal (received from the antenna 102) by demodulating the receive signal to recover the source information signal, by further processing (e.g., error decoding) the recovered source information signal, and by digitizing the recovered and further processed source information signal. Alternatively, if further processing of the recovered source information signal is not needed (e.g., the source did not error code the source information signal), then the demodulator circuit 120 can be configured to omit the further processing of the recovered source information signal. Furthermore, the modulator circuit 122 is configured to send sensor-patch information, such as a sensor-measurement value (e.g., temperature), sensor-patch status, or other sensor-patch data, to the source by modulating the source signal with the sensor-patch information; that is, in addition to providing power and carrying source information from the source to the sensor patch 100, the source signal also carries sensor-patch information from the sensor patch to the source. For example, the controller circuit 114 is configured to generate a digital sensor-patch-information signal that represents the sensor-patch information, and the modulator circuit 122 is configured to amplitude, frequency, or phase modulate the source signal with the sensor-patch information signal via the antenna 102; said another way, the modulator circuit 122 effectively modulates the carrier signal generated by the source with the sensor-patch information signal. For example, the modulator circuit 122 is configured to amplitude-load-modulate (ALM) the carrier signal generated by the source with the sensor-patch information signal. The controller circuit 114 or the modulator circuit 122 also can be configured to further process the sensor-patch information signal by, for example, error-encoding the sensor-patch information signal before the modulator circuit modulates the source signal. The source typically includes a demodulator circuit, which may be similar to the demodulator circuit 120, configured to recover the sensor-patch information by demodulating the source signal to recover the sensor-patch information signal, by further processing (e.g., error decoding) the recovered sensor-patch information signal, and by digitizing the recovered and further processed sensor-patch information signal. Alternatively, if further processing of the recovered sensor-patch information signal is not needed, then the source can be configured to omit the further processing of the recovered sensor-patch information signal. Therefore, the above-described configurations of the source and the sensor-patch 100 allow the source and sensor patch to communication with one another bidirectionally over a carrier signal that the source generates. To prevent the source and sensor-patch information signals from interfering with one another, the source and the sensor patch 100 can be configured to implement one or more conventional interference-prevention techniques. For example, the source and sensor patch 100 can be configured to implement time-division multiplexing such that the sensor patch 100 does not modulate the source signal with the sensor-patch information signal while the source is modulating the source signal with the source information signal, and the source does not modulate the source signal with the source information signal while the sensor patch is modulating the source signal with the sensor-patch information signal. In such an embodiment, the NFC source (e.g., a smart device such as a smart phone) may be the master and indicate, via the source information signal, when the sensor patch 100 can, and cannot, modulate the source signal with the sensor-patch information signal. Or, if the source and the sensor patch 100 are configured to implement frequency modulation, then the source and the sensor patch each can be configured to frequency modulate the carrier signal (which is generated by the source) at significantly different respective modulation frequencies. In addition, the communication circuit 106 is configured to receive, and to be powered by, the regulated supply voltage generated by the power-supply circuit 118.

The memory circuit 108 may include one or both of volatile and non-volatile memory, and is configured to receive, and to be powered by, the regulated supply voltage generated by the power-supply circuit 118. For example, the non-volatile memory can be configured to store circuit-configuration data for configuring one or more circuits of the sensor patch 100, or can be configured to store a set of program instructions that, when executed by the controller circuit 114, cause the controller circuit to operate as described herein for one or more embodiments. For example, while executing some or all of the stored instructions, the controller circuit 112 is configured to execute a sensor measurement and a reporting and notification cycle. The volatile memory may include registers and buffers configured for storing source information recovered by the demodulator circuit 120, and configured for storing sensor-patch information that the controller circuit 114 previously generated for transmission to the source via the modulator circuit 122 (i.e., the modulator circuit is configured to modulate, via the antenna 102, the source signal with the sensor-patch information as described above).

Each of the one or more sensors 110 is configured to sense a respective physical quantity or condition such as temperature of an object (e.g., a human forehead) to which the sensor patch 100 is attached, a level of ambient light to which the sensor patch is exposed, a level of humidity to which the sensor patch is exposed, or a linear movement (e.g., acceleration), angular movement (e.g., angular velocity), or a vibration (e.g., sound) that the sensor patch experiences, and to generate a respective analog or digital sense signal that represents a value of the sensed quantity or condition. For example, a sensor 110 can include a conventional thermistor circuit (not shown in FIG. 1) configured to sense a temperature and to generate a voltage or current having a magnitude, phase, or frequency that represents, or that is otherwise related to (e.g., proportional to, inversely proportional to), the value of the sensed temperature. The one or more sensors 110 can be configured to receive, and can be configured to be powered by, the regulated power signal that the power-supply circuit 118 is configured to generate, or can be configured to be powered by a signal generated by the controller circuit 114 (e.g., by a power-supply circuit 128 onboard the controller circuit).

The reporter, or indicator circuit 112 (also “indicator”) is configured to indicate locally, in response to the one or more sense signals generated by the one or sensors 110, a value of a physical condition or quantity that the one or more sensors 110 sense, or a range in which the value is located. The reporter circuit 112 includes one or more light-emitting diodes (LEDs), such as a red-green-blue (RGB) LEDs display 124, and can also include another reporter circuit 126, such as an alphanumeric display (e.g., a liquid-crystal display (LCD)), a sound generator, chromogenic ink, or a vibration (haptic) generator. The RGB LED display 124 is configured to generate a light having a color indicative of a range in which the value of the sensed physical condition or quantity is located. For example, one of the sensors 110 is a temperature sensor configured to sense a temperature of a human body while the sensor patch 100 is attached to a region (e.g., forehead) of the body. Considering that 98.6° Fahrenheit (° F.) is considered to be the normal body temperature of a healthy human, the RGB LED display 124 can be configured to generate blue light if the temperature that the sensor 110 senses is below 97.6° F., to generate a green light if the temperature that the sensor senses is within the range 97.6° F.-99.6° F. inclusive, and to generate a red light if the temperature that the sensor senses is greater than 99.6° F. Further to this example, the other reporter 126 can be an alphanumeric display configured to display the sensed temperature, for example, “97° F.” Or, the other indicator 126 can be a piezoelectric crystal configured to generate a sequence of sounds or vibrations that is indicative of the sensed temperature. For example, the piezoelectric crystal can be configured to generate a single sound or vibration if the temperature that the sensor 110 senses is below 97.6° F., to generate two sounds or vibrations if the temperature that the sensor senses is within the range 97.6° F.-99.6° F. inclusive, and to generate three sounds or vibrations if the temperature that the sensor senses is greater than 99.6° F. Or, the piezoelectric crystal can be configured to “play” a first tune if the temperature that the sensor 110 senses is below 97.6° F., to play a second tune if the temperature that the sensor senses is within the range 97.6° F.-99.6° F. inclusive, and to play a third tune if the temperature that the sensor senses is greater than 99.6° F. And it is understood that the LED display 124 and the other reporter 126 being “configured to” perform a respective function includes the controller circuit 114 being configured to cause the LED display and the other reporter to perform the respective function. The reporter circuit 112 can be configured to receive, and to be powered by, the regulated power signal that the power-supply circuit 118 is configured to generate, or can be configured to be powered by a regulated voltage signal generated by the controller circuit 114.

The controller circuit 114 is configured to communicate with, and to control, one or more other circuits and components of the patch sensor 100, in response to being configured with configuration data stored in the memory circuit 108 (e.g., in a non-volatile-memory section of the memory circuit), in response to executing program instructions stored in the memory circuit (e.g., in a non-volatile-memory section of the memory circuit), or in response to both being configured with configuration data and executing program instructions. The controller circuit 114 can include, for example, one or more microprocessors or microcontrollers, and also can include a power-supply circuit 128 disposed internal to the controller circuit and configured to generate a regulated power-supply signal for the controller circuit and for one or more other circuits and components of the sensor patch 100. For example, the power-supply circuit 128, which can be similar to the power-supply circuit 118, can be configured to receive the raw power signal from the energy-harvester circuit 116, and to convert the raw power signal into a regulated power-supply voltage.

Furthermore, the controller circuit 114 can be configured to “wake up” in response to receiving, from the energy-harvester circuit 116, the raw power signal having a steady voltage level that exceeds a threshold value, such as 1.8 V. For example, the controller circuit 114 can include a wake-up circuit (e.g., a power-on-reset (POR) circuit) configured to receive power from the power-supply circuit 128, which activates automatically in response to receiving the raw power signal having a steady voltage level that exceeds a threshold value.

After the power-supply circuit 128 begins to generate a regulated power-supply voltage signal, the controller circuit 114 is configured to activate other circuits within the controller circuit. For example, the POR circuit onboard the controller circuit can generate a reset signal having an enable value (e.g., a logic 1 or a logic 0) in response to the power-supply circuit 128 generating a regulated power-supply voltage signal having a voltage level above a threshold, and the other circuits can be configured to receive the reset signal and to commence operations in response to the reset signal having the enable value.

After activation of its circuitry, the controller circuit 114 is configured to load configuration data (if applicable) from the memory circuit 108, to configure itself in response to the configuration data, and to commence executing program instructions stored in the memory circuit.

The controller circuit 114 is configured next to receive, from the demodulator circuit 120, any source information that the demodulator circuit 120 recovered from the receive signal from the antenna 102, and to act on the received source information.

For example, if the source information includes a command to measure and to display a temperature, then the controller circuit 114 first causes at least one of the one or more sensors 110 to sense a temperature and to generate a corresponding sense signal.

Next, the control circuit 114 receives and processes the sense signal (e.g., the control circuit 114 may include an analog-to-digital converter (ADC), and may cause the ADC to convert the sense signal from an analog signal to a digital signal if the sense signal is not already in digital form), and, in response to the sense signal, determines a value of the temperature sensed by the at least one sensor 110.

Then, the control circuit 114 causes the indicator circuit 112 to generate an indication of the determined value of the sensed temperature. For example, the control circuit 114 may cause the reporter display 126 to display a numeric, or alphanumeric, determined value of the sensed temperature, or may cause the LED 124 to generate a color indicative of a range within which the determined value of the sensed temperature lies. Further in example, if the determined value of the sensed temperature is 99° F., then the controller circuit 114 causes the display 126 to display “99° F.,” or causes the LED 124 to generate a green light to indicate that the value of the sensed temperature lies within a range from 97.6° F. to 99.6° F. inclusive.

The controller circuit 114 also may generate a sensor-patch-information signal that represents the determined value of the sensed temperature, send the sensor-patch-information signal to the modulator circuit 122, and cause the modulator circuit to send the determined value of the sensed temperature to the source smart device (not shown in FIG. 1) via the antenna 102 by modulating the source signal with the sensor-patch-information signal. Consequently, the source smart device receives, and can display, store, and otherwise process the determined value of the sensed temperature.

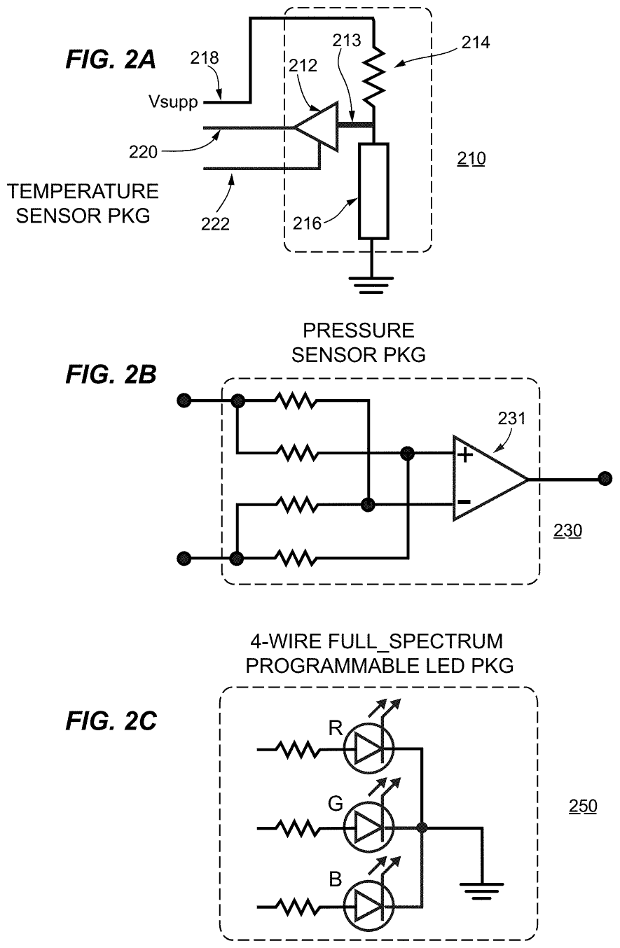

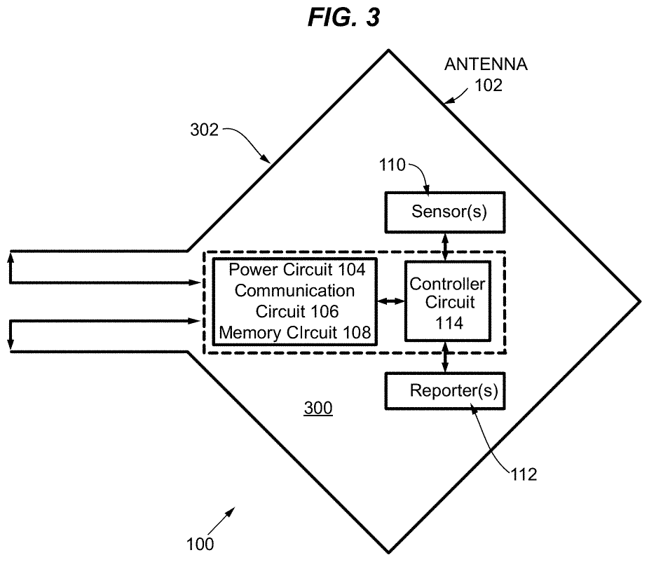

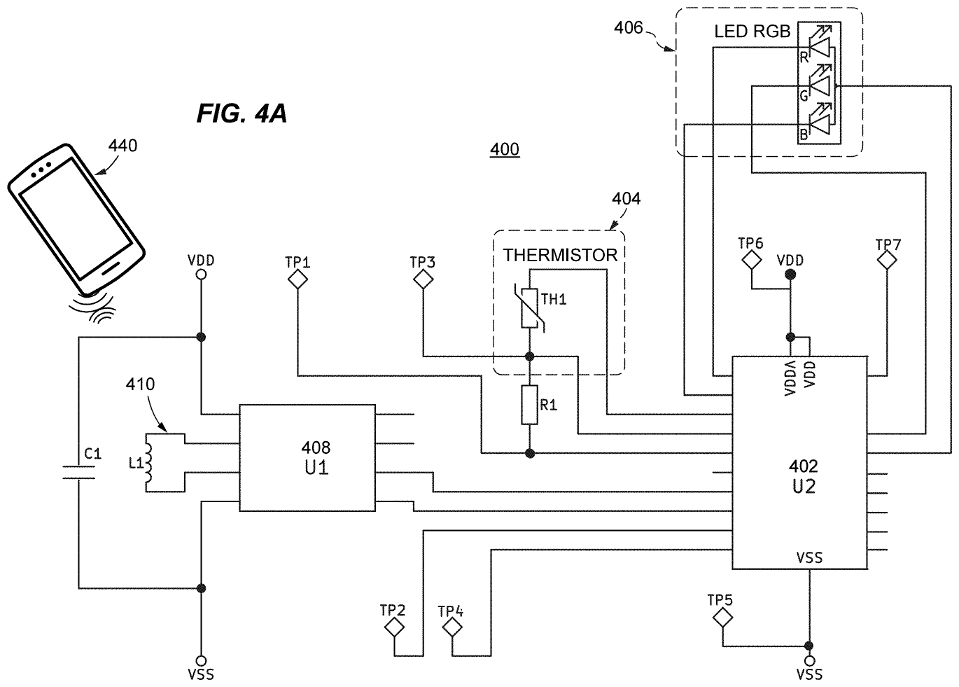

Still referring to FIG. 1, alternate embodiments of the sensor patch 100 are contemplated. For example, although described as being separate, the circuits 104, 106, 108, and 114, the sensors 110, the reporter circuit 112, and the controller circuit 114 can be disposed on a single integrated circuit 130 such as a system on a chip (SOC), or can be disposed on two or more integrated circuits. Further in example, where the controller circuit 114 is disposed on a separate integrated circuit, then the controller circuit may need to have firmware “flashed” to internal memory during manufacture and test, and may call for a respective bypass capacitor (not shown in FIG. 1) between each of its one or more power-supply nodes and ground; but where the controller circuit, power circuit 104, and communication circuit 106 are integrated on a same integrated circuit, then such integrated circuit may be configured to allow omission of the firmware flash to internal memory during manufacture and test and of one or more of the bypass capacitors. Furthermore, the sensor patch 100 can include fewer or more circuits or components than those described above. Moreover, although one of the one or more sensors 110 is described as being a temperature sensor, the one or more of the sensors can be any suitable type of sensor, such as acoustic (e.g., piezoelectric, microphone), optical (e.g., photocell for reflected light, CMOS pixel array), and chemical (e.g., to detect substances in sweat) sensors, multi-axis accelerometers, multi-axis gyroscopes, and microelectromechanical (MEMs) devices such as MEMs cantilevers. For example, a displacement sensor can be configured to sense a heartbeat in a peripheral artery of a human by detecting a transient increase in displacement. The controller circuit 114 can readily analyze characteristics of the displacement to calculate heart rate, but also can analyze other characteristics to determine cardiac output such as left-ventricular ejection volume. And in extreme cases of congestive heart failure, the sensor patch 100 can be configured to report a local alarm state and to notify a remote dispatcher, a nursing station, or a bedside monitor that assistance is required. In addition, although described as being a microcontroller or microprocessor, the controller circuit 114 can be, or can otherwise include, any suitable circuit, such as a digital signal processor (DSP), an application specific integrated circuit (ASIC), a field-programmable gate array (FPGA) or other programmable logic device, discrete gate or transistor logic, discrete hardware components, or any combination thereof designed to perform the functions described herein. Furthermore, although described as extracting power from, and communicating via, an NFC signal, the sensor patch 100 can be configured to extract power from, and to communicate via, a radio-frequency-identification (RFID) signal. Moreover, the LED 124 may be omitted, the reporter display 126 may be a chromogenic ink or a similar device that has a color corresponding to a temperature of an object to which the sensor patch 100 is in contact, and the sensor patch is configured to send a value of a sensed or determined temperature to the smart device generating a wireless source signal for numerical display of the value; therefore, the sensor patch 100 is configured to indicate a sensed or determined temperature with little or no power draw by the reporter display. In addition, the following references, which disclose NFC and RFID circuitry and techniques that may be suitable for use in, or in conjunction with, the sensor patch 100, are incorporated by reference: A 13.56 MHz Passive NFC Tag IC in 0.18-μm CMOS Process for Biomedical Applications, Lu et al., 978-1-4673-9498-7/16, IEEE 2016; Near Field Communication (NFC) Technology and Measurements, Minihold, version 1MA182_5e, June 2011; TN1216 Technical Note ST25 NFC Guide, DocID027940 Rev 2, October 2016; Near Field Communication (NFC)—A Technical Overview, Motlagh, University of Vaasa, 5 Nov. 2015; AN11755, PN7150 Antenna Design and Matching Guide, NXP, Rev. 17, 10 Jul. 2019; NFC Reader Design: How to build your own reader, NXP MobileKnowledge, February 2015; U.S. Patent App. Pub. 2018/0110018; U.S. Patent App. Pub. 2014/0349572; U.S. Pat. Nos. 8,983,374; 9,014,734; 9,345,050; 9,379,778; 9,613,747; 8,818,267; 8,914,061; 8,018,344; 8,050,651; U.S. Patent Pub. 2012/0083205; U.S. Pat. Nos. 9,225,372; 9,236,658; U.S. Patent Pub. 2007/0026825; U.S. Pat. No. 9,496,925; U.S. Patent Pub. 2009/0011706; and U.S. Pat. No. 9,782,082. Moreover, features of embodiments described in conjunction with FIGS. 2A-37 may be applicable to the patch sensor 100 of FIG. 1.

FIG. 2A is a schematic diagram of a temperature sensor 210, which is suitable for use as at least one of the one or more sensors 110 of the sensor patch 100 of FIG. 1, according to an embodiment. The temperature sensor 210 includes an amplifier 212 having an input node 213, a resistor 214, a thermistor 216, a supply node 218 configured to receive a supply voltage Vₛᵤₚₚ (e.g., from the controller circuit 114 of FIG. 1), an output node 220 coupled to a sense-signal input node of the controller circuit, and a control node 222.

In operation, the controller circuit 114 of FIG. 1 generates, on the supply node 218, the supply voltage Vₛᵤₚₚ, and generates, on the control node 222, a control signal having an enable value (e.g., a logic 1) that enables the amplifier 212 to amplify (e.g., with unity gain) a voltage on the input node 213 and to generate an output voltage on the output node 220; alternatively, the input node may be coupled to receive the regulated supply voltage Vₛᵤₚₚ from the power-supply circuit 118 of FIG. 1. The resistor 214 and the thermistor 216 form a voltage divider that generates, at the input node 213, a temperature-dependent sense voltage

$${}_{sense} ( ) = \frac{{R}_{TR} ( T )}{{R}_{R} + {R}_{TR} ( T )} \cdot {}_{supp} ,$$

where R_{TR}(T) is the temperature-dependent resistance of the thermistor 216, R_{R} is the resistance of the resistor 214, and T is temperature in units of Kelvin; for example, the resistance R_{TR}(T) of the thermistor increases or decreases with increasing temperature, and decreases or increases with decreasing temperature. Because R_{TR}(T) depends on, i.e., is a function of, the temperature T of the thermistor 216, the sense voltage Vₛₑₙₛₑ(T) also is a function of the temperature T of the thermistor. That is, the sense voltage Vₛₑₙₛₑ(T) is related to the temperature that the thermistor 216 experiences, or senses; for example, the sense voltage Vₛₑₙₛₑ(T) increases or decreases with increasing thermistor temperature, and decreases or increases with decreasing thermistor temperature. The amplifier 212 amplifies Vₛₑₙₛₑ(T), for example with a gain of approximately unity (i.e., the amplifier 212 operates as a buffer), and provides the amplified Vₛₑₙₛₑ(T) to the controller circuit 114 via the output node 220.

Because the relationship between the resistance of, and the temperature experienced by, the thermistor 216 may change from thermistor to thermistor, and because the resistance of the resistor 214 may change from resistor to resistor and with temperature, during manufacture of the sensor patch 100 (FIG. 1), a technician or automatic-testing setup can calibrate Vₛₑₙₛₑ(T) to temperature sensed by the thermistor 216, and can store, in the memory circuit 108 (FIG. 1), a calibration algorithm or look-up table (LUT) that relates Vₛₑₙₛₑ(T) to the sensed temperature. An example calibration algorithm is x·Vₛₑₙₛₑ(T)=temperature in degrees Fahrenheit (F), where x can be a scalar, a function of temperature T, or other type of factor, the value of which is determined during a calibration procedure and is stored in the memory circuit 108. Further in example, if x=100 and Vₛₑₙₛₑ(T) equals 1.0 V, then the controller circuit 114 determines that the value of the temperature experienced, and thus sensed, by the temperature sensor 210, is equal to 100·1.0 V=100° F. Alternatively, the memory circuit 108 can store an LUT having a respective value of temperature for each of a number of values of Vₛₑₙₛₑ(T). For example, if Vₛₑₙₛₑ(T)=1.0 V, then the controller circuit 114 retrieves, from the LUT in the memory 108, the determined value of the sensed temperature corresponding to 1.0 V. If Vₛₑₙₛₑ(T) lies between two voltage values for which the LUT stores respective values of sensed temperature, then the controller circuit 114 can determine the value of the sensed temperature according to any suitable interpolation algorithm. In addition, the controller circuit 114 can determine the value of temperature that the sensor 210 senses by both using an LUT and an algorithm.

Still referring to FIG. 2A, alternate embodiments of the temperature sensor 210, the calibration procedure, the calibration algorithm, and the LUT are contemplated. For example, although described as a constant, x can be a variable that is a function of temperature or on another quantity or condition. Furthermore, although described as being an analog temperature sensor, the temperature sensor 210 can be a digital temperature sensor, which can eliminate the thermistor 216 and calibration during manufacture or test. Moreover, features of embodiments described in conjunction with FIGS. 1 and 2B-37 may be applicable to the temperature sensor 210 of FIG. 2A.

FIG. 2B is a schematic diagram of a solid-state resistor array configured as a pressure sensor 230, which is suitable for use as at least one of the one or more sensors 110 of the sensor patch 100 of FIG. 1, according to an embodiment. The resistor network disclosed herein is configured to respond to local deformation by outputting a differential voltage from an amplifier 231, which can be or include, for example, an operational amplifier. By positioning the resistor network over a pulse such as radial or a carotid pulse, the pulsatile deformations caused by a beating heart can be sensed and a related sense signal can be generated by the pressure sensor 230 and sent to the controller circuit 114.

Sensor devices, such as the sensor patch 100 (FIG. 1) having a solid-state pressure sensor, such as the pressure sensor 230, may give an indication of barometric pressure but not a pressure differential resulting from compression or deformation of the solid-state device. In other embodiments, sensors that respond to deformation are known to include capacitive and inductive pressure sensors having deformable proximity between two opposing electrically interactive surfaces or elements. Magnetic Hall effect sensors also can be miniaturized to generate a voltage according to minute changes in the separation of two magnetically responsive elements. MEMs sensors built using etch methods derived from nanotechnology also can be used to generate a sensor signal from a deformation.

An embodiment has a strain gauge or a load cell. Thus, a variety of sensors can be adapted for use as at least one sensor of the one or more sensors 110 of the sensor patch 100 of FIG. 1.

Still referring to FIG. 2B, alternate embodiments of the pressure sensor 230 are contemplated. For example, although described as being an analog pressure sensor, the pressure sensor 230 can be a digital pressure sensor. Furthermore, features of embodiments described in conjunction with FIGS. 1, 2A, and 2C-37 may be applicable to the pressure sensor 230 of FIG. 2B.

FIG. 2C is a schematic diagram of an RGB LED circuit 250, which is suitable for use as the RGB LED 124 of FIG. 1, according to an embodiment. Three LEDs, each of a different color (e.g., red (R)/green (G)/blue (B)) are assembled in a package having four leads, a respective input lead for each LED and a common reference, e.g., ground, lead. By varying the respective voltage on each of the input leads, and, therefore, varying the respective current through, and intensity (e.g., brightness) of, each of the diodes while active, the controller circuit 114 (FIG. 1) can cause the circuit 250 to generate a rainbow of colors. Alternatively, the controller circuit 114 can be configured to activate fewer than all of the LEDs at one time. For example, as described above in conjunction with FIG. 1, the controller circuit 114 can be configured to activate the red LED, and to deactivate the green and blue LEDS, to indicate a determined temperature that is greater than 99.6° F., to activate the green LED, and to deactivate the red and blue LEDs, to indicate a determined temperature that is within a range of 97.6° F.-99.6° F. inclusive, and to activate the blue LED, and to deactivate the red and green LEDs, to indicate a determined temperature that is less than 97.6° F.

The LED circuit 250 may be obtained as a wire-bonded package, as a solder-bump package, or as a flip-chip package, and may have an orientation normal to a substrate surface, lateral to a substrate surface, or even inverted such that the LED emission is directed through the substrate surface (assuming the substrate surface is translucent or transparent).

Still referring to FIG. 2C, alternate embodiments of the RGB LED 250 are contemplated. For example, although described as allowing the active/inactive status and the respective brightness of each LED to be controlled in an analog manner, the RGB LED 250 can be configured to allow the active/inactive status and the respective intensity of each LED to be controlled in a digital manner.

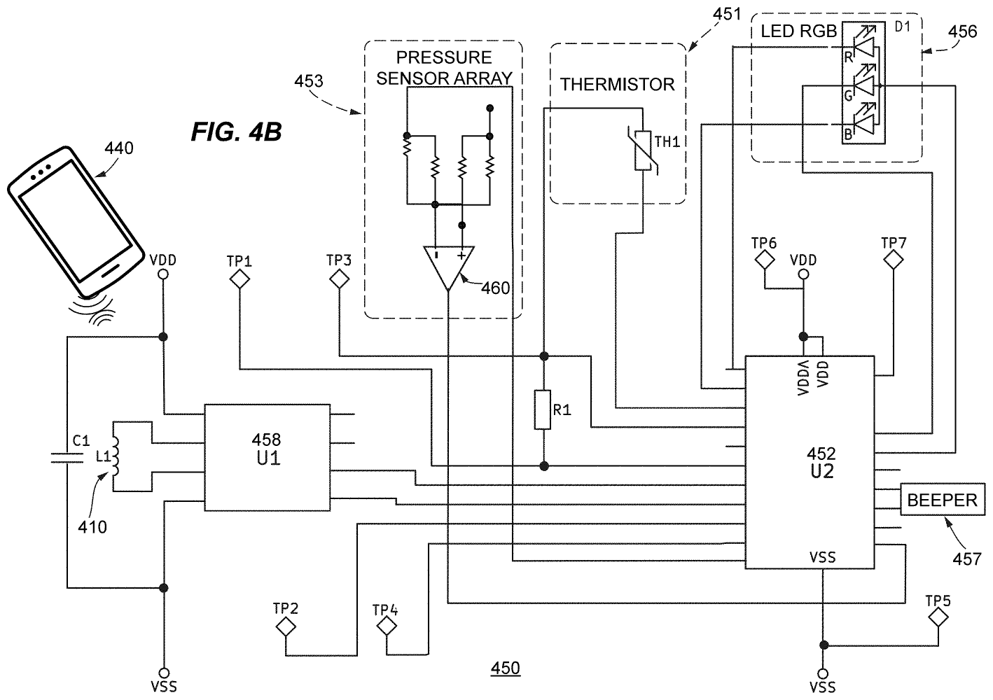

FIG. 3 is a plan view of the patch sensor 100 of FIG. 1, according to an embodiment in which the antenna 102 is, or otherwise includes, a loop antenna, and in which the power circuit 104, communication circuit 106, memory 108, the one or more sensors 110, the reporter circuit 112, and the controller circuit 114 are disposed within a region (e.g., area, volume) 300 bounded by at least one loop 302 of the antenna. Such an efficient layout allows the antenna 102 to have a size (e.g., loop diameter or loop area) sufficiently large for extracting power from the source signal from the NFC source smart device (not shown in FIG. 3) and also allows the overall size (e.g., diameter) of the sensor patch 100 to be sufficiently small for attachment to an object such as a child's forehead.

Still referring to FIG. 3, alternate embodiments of the sensor patch 100 are contemplated. For example, features of embodiments described in conjunction with FIGS. 1-2C and 4-37 may be applicable to the sensor patch 100 of FIG. 3.

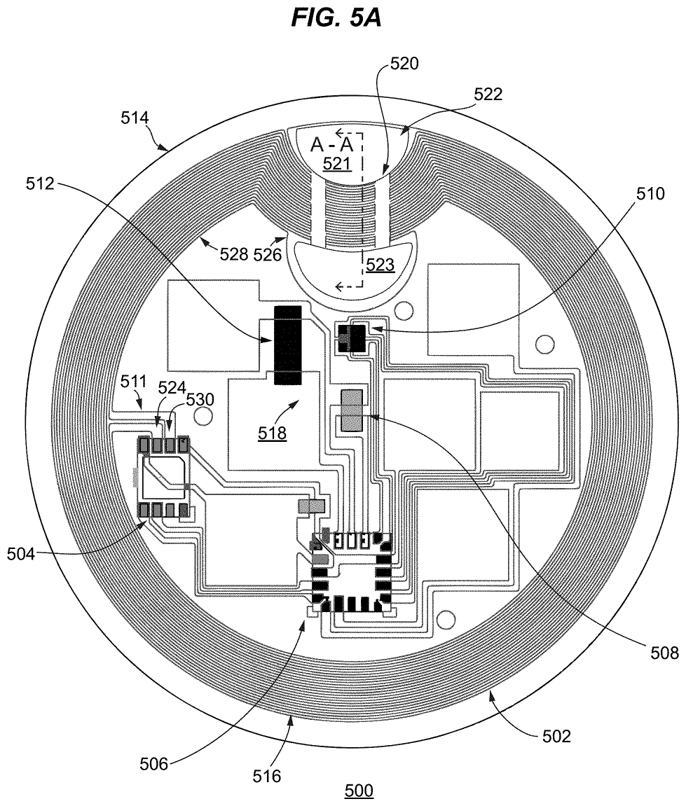

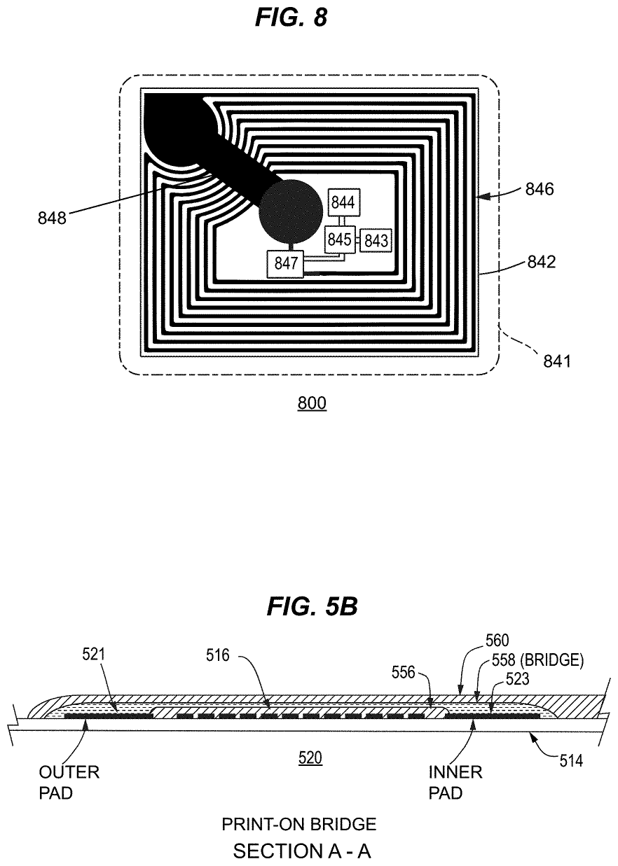

FIG. 4A is a schematic diagram of system including a sensor patch 400 and a smart device 440 configured to power, communicate with, and control the sensor patch, according to an embodiment. The sensor patch 400 includes an integrated controller circuit 402 (U2) and a peripheral temperature sensor 404 that includes a thermistor TH1) with a local display 406 (here an RGB LED). The sensor patch 400 also includes a communication-and-power circuit 408 (U1) with an antenna 410, represented as an inductor having an inductance L1 configured for NFC power harvesting and bidirectional exchange of data between the sensor patch 400 and the smart device 440, and includes a power-supply bypass capacitor C. As described above, information is transmitted by modulation and demodulation of a carrier wave in the NFC signal transmitted by the smart device 440.

The antenna 410 is a loop antenna, and the communication-and-power circuit 408 includes circuitry for power harvesting and generation of a regulated power signal having a stable (e.g., regulated) voltage level. In an embodiment, the controller circuit 402 includes a POR circuit (not shown in FIG. 4A) configured to enable the controller circuit in response to the voltage level of the regulated power signal equaling or exceeding a threshold voltage; the communication-and-power circuit 408 also may include such a POR circuit. Or the controller circuit 402 may include a power-supply circuit (not shown in FIG. 4A) configured to generate a regulated supply voltage in response to a raw power signal from the communication-and-power circuit 408, and may include a POR circuit configured to enable the controller circuit in response to the voltage level of this internally generated regulated supply voltage equaling or exceeding a threshold voltage.

The communication-and-power circuit 408 also includes circuits for modulation and demodulation as described above in conjunction with FIG. 1. And one or both of the communication-and-power circuit 408 and the controller circuit 402 include a clock, random-access (RAM) memory, non-volatile memory such as electrically erasable and programmable read-only memory (EEPROM), and pin-out connections to a sensor module or modules 404, optionally multiplexed or on an I²C bus to permit sequential measurements with different classes of sensors including analog and digital sensor outputs.

Antennas and circuitry for wireless power transfer by harvesting near-field RF power are designed so that the antenna size and impedance are sufficient to power the sensor patch 400 from the near-field source signal transmitted by the smartphone 440. Wireless power transfer using capacitive coupling, inductive coupling, or magnetic resonant coupling are known, but near-field wireless power transfer to a sensor circuit embedded in an adhesive patch has not been combined so that the sensor patch reports sensor data directly via a local reporter display and also can notify at least one remote device (such as the smart device 440, which powers the sensor circuit) wirelessly, with the option of forwarding the notification to a broad area network or a remote workstation. For example, the programmable smart device 440 having a suitable NFC antenna and signal generator is set up with a software application (e.g., an “app”) that when run, renders the smart device suitable to energize the sensor patch 400 and to collect data from the sensor patch via the communication-and-power circuit 408. Generally, power is provided, and data are exchanged simultaneously, by a near-field source signal generated by the smartphone 440, having a suitable center frequency such as 13.56 MHz, and, when amplitude modulated with a data signal, having two sidebands at suitable frequencies such as 12.71 MHz and 14.41 MHz, respectively.

The sensor patch 400 is configured to keep power consumption at, or less than, 300 microwatts (μW) in an embodiment. With the thermistor 404, the sensor patch 400 is configured to achieve a temperature accuracy in an approximate range of 0.05° C. to 0.1° C. in the approximate temperature range of 35° C. to 40° C. For measuring body temperature in a clinical environment, this accuracy and reproducibility is comparable with conventional ear- and forehead-temperature-measurement systems and is better than the conventional wet-bulb thermometers.

For inanimate objects, in an embodiment where the monitored parameter is temperature, the smart device 440 can be configured to compare a history of temperatures encountered in earlier measurements during storage or transmit, to a previously established range of acceptable values. The device 440 then can store selected data, such as data representative of (1) the occurrence of excursions outside of the acceptable range, (2) times of occurrence of the first cross-over and last cross-over from acceptable values to overage and/or underage (measured with respect to the acceptable value range) for excursions outside the acceptable range, (3) times of occurrence of and magnitude of the extreme values during excursions outside the acceptable range, and (4) the number of out-of-range excursions. Generally, the information may be retrieved in two ways: 1) visual display upon user-activation of a display device; or 2) up-loading to an external computer device. The sensor patch 400 also may be configured to report a temperature excursion by making a direct display to the user (not shown in FIG. 4A). The display device can be a buzzer, an LED light, a haptic display, or an electrochromic label with color-coded reporter, for example.

The sensor patch 400 is a passive device with no power source of its own. Accordingly, to use the sensor patch 400, a user brings the NFC antenna of the smart device 440 into proximity with the antenna 410 of the sensor patch 400. Power is extracted from an NFC receive signal generated by the antenna 410 in response to receiving an NFC source signal from the smart device 440. That is, the sensor patch 400 harvests, from the smart device 400, power to operate the sensor-patch electronics.

In NFC data transfer by standard interfaces, the underlying layers of NFC technology follow the normal ISO standards. For example, the data-transfer rate may be either 106, 212, or 424 kbps. The software application that the smart device 440 runs sets up the initial communication speed, but the speed may be changed later depending upon the communication environment and the requirements. Compatibility between the smart device 440 and the sensor patch 400 is established during initial inductive coupling of the antenna 410 and the antenna (not shown in FIG. 4A) of the smart device. For example, the smart device 440 generates a source magnetic field (e.g., a source signal) with a loop antenna (not shown in FIG. 4A), and one places the smart-device antenna close enough to the sensor-patch antenna 410 so that the smart-device and sensor-patch antennas are inductively (e.g., magnetically) coupled and the source magnetic field induces a receive current signal i in the sensor-patch antenna. The receive current induces, across nodes 460 and 462 of the antenna 410, a voltage V_{receive}=Ldi/dt. For example, if di/dt is sinusoidal, then V_{receive} also is sinusoidal, and if di/dt is a sinusoidal signal that is modulated with an information signal, then V_{receive} also is a sinusoidal signal that is modulated with an information signal. Said another way, V_{receive} differs from di/dt only by the scalar value L of the inductance of the antenna 410. The sensor patch 400 includes a memory circuit, the size of which may be sufficient to load a measurement from an analog-to-digital converter (ADC) or from a digital sensor chip and deliver that measurement to an encoder for broadcast through the NFC communications circuit 408.

The communications circuit 408 is configured to broadcast (effectively via modulation of the source signal) an NFC signal with a radio device identifier (UID) that uniquely identifies the sensor patch 400. Messages may range from 8 bits to 128 bits for most applications, but for higher accuracy, such as for more complex pressure data, serial frames may be broadcast.

Still referring to FIG. 4A, alternate embodiments of the sensor patch 400 are contemplated. For example, features of embodiments described in conjunction with FIGS. 1-3 and 4B-37 may be applicable to the sensor patch 400 of FIG. 4A.

FIG. 4B is a schematic diagram of a system that includes a sensor patch 450 and the smart device (e.g., a smart phone) 440, according to an embodiment. The sensor patch 450 includes an integrated controller circuit 452, a temperature sensor 451, a pressure-sensor array 453, and a communication-and-power circuit 458. Temperature or pressure is selected for reporting according to the programming or according to commands from the smart device 440 to the controller circuit 452. Local display of sensor output is enabled using an RGB LED 456 or an optional beeper 457. The communication-and-power circuit 408 (U1) and antenna circuit 410 are configured for power harvesting and for bidirectional exchange of data by modulation and demodulation of an NFC signal transmitted by the smart device 440.

The two sensors 451 and 453, temperature and pressure, are configured for independent operation. Both are analog sensors but may be supplied with an integrated digital-conversion capacity. The pressure sensor 453 is positioned on a flexible circuit membrane so that deformation of the circuit membrane and resistor network of the pressure sensor array is sufficient to generate a voltage difference, amplified by the op-amp and then processed by the controller circuit 452.

Still referring to FIG. 4B, alternate embodiments of the sensor patch 450 are contemplated. For example, features of embodiments described in conjunction with FIGS. 1-4A and 5-37 may be applicable to the sensor patch 450 of FIG. 4B.

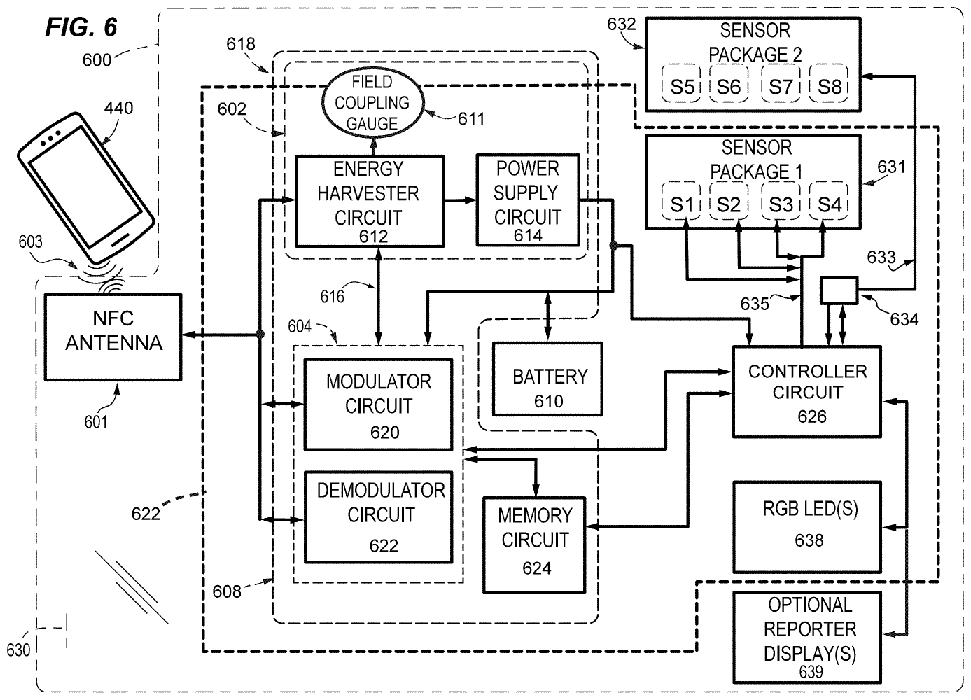

FIG. 5A is a plan view of a sensor patch 500, which can include circuitry similar to the circuitry of the sensor patch 100 of FIG. 1, according to an embodiment. The sensor patch 500 measures less than 2.0 centimeters (cm) in diameter and less than 1.0 millimeters (mm) in thickness, and includes an antenna 502, communication-and-power circuit 504, controller circuit 506, RGB LED 508, thermistor 510, thermal-sensor resistor 512, and a flexible substrate 514.

The antenna 502 includes conductive loops 516 disposed around a periphery of the flexible substrate 514.

The communication-and-power circuit 504 includes a power circuit that can be similar to the power circuit 104 of the sensor patch 100 of FIG. 1, includes a communication circuit that can be similar to the communication circuit 106 of the sensor patch 100 of FIG. 1, and may include a memory circuit similar to the memory circuit 108 of the sensor patch 100 of FIG. 1.

The controller circuit 506 can be similar to the controller circuit 114 of the sensor patch 100 of FIG. 1.

The RGB LED 508 can be similar to the RGB LED 124 of the sensor patch 100 of FIG. 1.

The thermistor 510 and the thermal-sensor resistor 512 form a temperature sensor 518, which can be similar to the temperature sensor 210 of FIG. 2A.

The flexible substrate 514 is formed from a translucent and flexible plastic such as polyethylene terephthalate (PET), polycarbonate (PC), nylon, a fluoropolymer, polyimide (e.g., KAPTON®), or other higher dielectric plastic. The substrate 514 is coated with an aluminum or copper film, and then is masked, etched, and otherwise processed in a conventional manner to form the antenna loops 516, other conductive traces, conductive pads, and conductive connection points that interconnect the components of the sensor patch 500.

The substrate 514 includes a crossover or bridge 520 over which a portion of the antenna 502 is disposed. An exemplary “print-on bridge” is shown in FIG. 5B as described below, referring to section A-A. The bridge 520 allows a connection of inner connection pad 523 to an outer node 522 of the antenna to cross under or over the antenna loops 516 without “short circuiting” any two or more of the antenna loops together. A first inner node of the antenna is coupled to a first node 524 of the communication circuit 504. From the first node 524, the continuous conductive trace that forms the loops 516 of the antenna 502 winds around the periphery of the substrate 514 to the outer node 522 of the antenna, and the bridge 520 includes an outer conductive landing 521 coupled to the outer node 522, and an inner conductive landing 523 coupled to a second inner node 526 of the antenna 502. A conductor (not visible in FIG. 5A, see FIG. 5B) coupled to the outer landing 521 traverses the bridge 520 over (or under) the lines of the antenna loops 516 to the inner conductive landing 523; the bridge is configured to insulate, electrically, the conductor from the antenna loops over or under which the conductor traverses. And a portion (528:511) of an antenna loop 516 electrically couples the second inner node 526 to a node 530 of the communication circuit 504. FIG. 5B is a cross-section of the bridge 520 taken along lines A-A.

A “chip” side (i.e., the side on which the components are mounted (the chip side is facing out of the page of FIG. 5A) of the substrate 514 can be sealed from water, salt, corrosion, and other impurities, contamination, and degradation by a liquid-impermeable protection film or layer, such as a passivation layer, formed over the chip side of the substrate.

And over the film is disposed a non-conductive adhesive layer (e.g., polyacrylate, methacrylate, acrylamide, or a silastic gel layer).

A release backing may also be applied over the adhesive layer for protecting the adhesive from contaminants and for removal just prior to adhering the sensor patch 500 to an object like a forehead of a human subject.

Furthermore, the sensor patch 500 can be disposable and, as described above in conjunction with FIG. 1, needs no battery or other onboard power source because the sensor patch is configured to be powered entirely by an NFC emission from a smart device, such as a smart phone, while an NFC antenna of the smart device is held in proximity (e.g., 0-12 inches) with the antenna 502.

Still referring to FIG. 5A, alternate embodiments of the sensor patch 500 are contemplated. For example, one or more of the protection layer, adhesive layer, and release backing may be disposed over a non-chip side of the substrate 514. Furthermore, although shown as having two terminals, the antenna 502 can have three or more terminals; for example, the antenna can have a center tap (in the center of the inner and outer terminals, where the center is located at one half the linear spiral distance between the inner and outer terminals). Moreover, features of embodiments described in conjunction with FIGS. 1-4B and 6-37 may be applicable to the sensor patch 500 of FIG. 5A.

FIG. 5B is a cutaway side view of the bridge 520 of FIG. 5A taken along lines A-A in FIG. 5A, according to an embodiment. The bridge 520 is configured so that the patch sensor 500 can be constructed with components and one or more layers of metal printed on only one side of the substrate 514. Such a single-side construction can be less complex and less expensive than a dual-side construction in which components and metal layers are on both sides of the substrate 514.

The antenna loops 516, the outer conductive landing 521 (“outer pad”), and the inner conductive landing 523 (“inner pad”) are disposed over the substrate 514 in a first conductive layer (solid black) of, for example, a metal such as copper.

A first electrical insulator layer 556 is disposed over the antenna loops 516 and respective portions of the outer and inner landings 521 and 523. The insulator layer 556 may be made from any suitably flexible dielectric material such as polyimide.

A second electrically conductive layer 558 is disposed over the outer conductive landing 521, the inner conductive landing 523, and the first insulator layer 556, and electrically couples the outer conductive landing to the inner conductive landing. The second conductive layer 558 may be made from any suitable electrically conductive material such as silver.

And a outer electrical insulator layer 560 is disposed over the second conductive layer 558 and the outer and inner conductive landings 521 and 523. The insulator layer 560 can be made from any suitably flexible dielectric material such as an epoxy, polyimide, or Al₂O₃, for example.

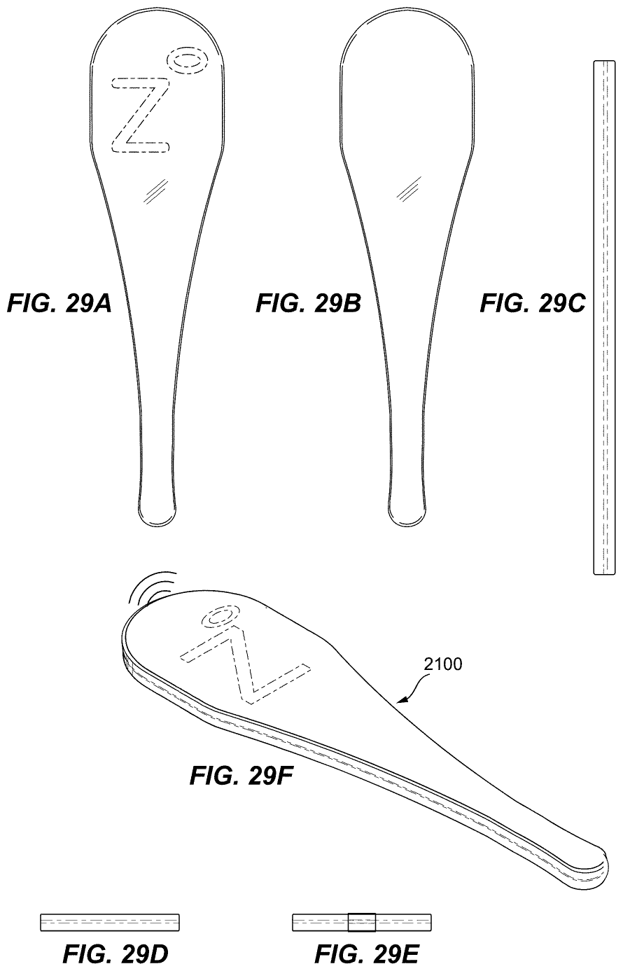

Still referring to FIG. 29, a method for forming the sensor patch 500, including the bridge 520, according to an embodiment, includes steps for:

First, the conductive layer (solid black) is formed over the substrate 514. For example, a layer of copper is conventionally formed over the substrate 514 to a thickness in an approximate range of 1-20 thousandths of an inch (mils).

Next, the conductive layer is conventionally patterned and etched to form the antenna loops 516, the outer and inner landings 521 and 523, and the traces and component pads (see FIG. 5A).

Then, the insulator layer 556 is conventionally formed over the antenna traces so as to leave respective portions of the outer and inner landings 521 and 523 exposed. For example, a suitable insulator material such as an epoxy, polyimide, or Al₂O₃, is printed over the antenna loops.

Next, the second conductive layer 558 is conventionally formed over the insulator layer 2902 and respective exposed portions of the outer and inner landings 521 and 523 such that the second conductive layer electrically couples the outer and inner landings to one another. This is the bridge 558. For example, a suitable metal such as silver, in a suitable form such as flakes, is printed over the insulator layer 556 and the respective exposed portions of the other and inner landings 521 and 523.

Then, the outer insulator layer 560 is conventionally formed over the second conductive layer 558 and respective exposed portions of the outer and inner landings 521 and 523, and protects the second conductive layer and the covered portions of the outer and inner landings. For example, a suitable insulator material such as an epoxy, polyimide, or Al₂O₃ is printed over the second conductive layer 558.

Referring to FIGS. 5A and 5B, alternate embodiments of the sensor patch 500 are contemplated. For example, the loops 516 of the antenna may be disposed over the conductive layer 558 in the bridge 520. Furthermore, features of embodiments described in conjunction with FIGS. 1-4B and 6-37 may be applicable to the sensor patch 500 of FIG. 29.

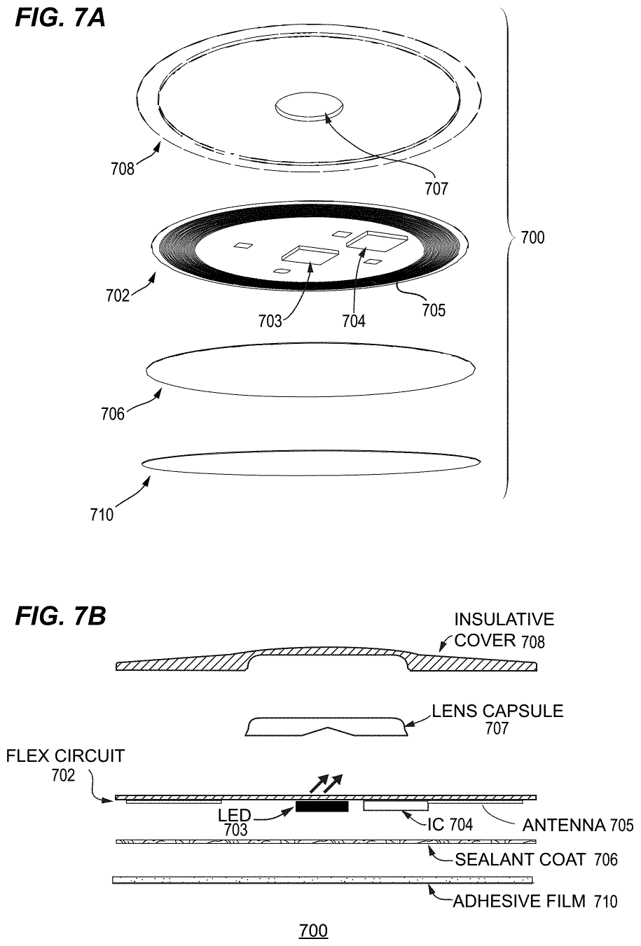

FIG. 6 is a diagram of a system that includes a smart device, such as a smartphone 440, and a sensor patch 600, according to an embodiment. The sensor patch 600 is configured for passive or active operation: that is, in passive mode, the sensor patch is inactive until the smartphone 440 transmits a source NFC signal 603, an NFC antenna 601 of the sensor patch 600 receives the source NFC signal and converts the source NFC signal into a receive NFC signal, and a power circuit 602 extracts, from the receive NFC signal, power to operate the sensor patch. In active mode, the sensor patch is powered by battery 610.

The NFC antenna 601 is configured to couple the receive NFC signal to a power circuit 602, which is configured to extract power from the receive NFC signal, and a communication circuit 604, which is configured to perform bidirectional NFC communications between the smart device 440 and the sensor patch 600. Power harvesting and data exchange can occur simultaneously. While the ability to exchange data can be dependent on an ongoing power supply from the source NFC signal generated and transmitted by the smart device 440, the sensor patch 600 includes a battery 610 (a capacitor, a rechargeable laminar battery, a printed battery, for example), which is configured, at least under some operating conditions, to allow the sensor patch 600 to operate for a period during which the antenna 601 does not receive the source NFC signal 603. Power harvested is split so that at least a part of the available energy is directed to battery so that it can be discharged if the source signal 603 is interrupted. The battery 610 is configured to power some or all of the circuits and other components of the sensor patch 600, for a duration related to the charge capacity or capacitance, in units of Amperes (A) or Farads (F), and the load that the circuits and other components of the sensor patch present to the battery.

The power circuit 602 includes an energy-harvester circuit 612 and a power-supply circuit 614. Together, the circuits 612 and 614 include a half- or full-wave rectifier, a voltage regulator, and a circuit (e.g., a POR circuit) configured to indicate when a supply voltage generated by the power-supply circuit 614 equals or exceeds a threshold voltage (e.g., 1.1 V). The power-supply circuit 614 is configured to power, with the supply voltage, at least the power circuit 602, the communication circuit 604, and a controller circuit 626 (e.g., a microprocessor or microcontroller that can include one or more of a memory cache, configurable (e.g., with configuration data such as firmware) logic circuitry, and a clock circuit). In some instances the power supply circuit can include a switching regulator, as will be described below, for dual power use.

The power circuit 602 may include a field-coupling gauge 611, which is configured to determine and report a position of the smart device 440 relative to the sensor patch 600 at which NFC power transfer from the smart device to the sensor patch is suitable, or even is highest. The field-coupling gauge 611 can cause the sensor patch 600 to signal suitable positioning of the smart device 440 by generating a steady tone or by other means to guide a user to maintain the indicated position. Note that the energy-harvester circuit 612 can include a bidirectional link 616 with the communication circuit 604, and the link may be useful for initial setup of a radio link during power up of the sensor patch 600 in passive mode.

As stated above, the energy-harvester circuit 612 and power-supply circuit 614 supply power to the communication circuit 604, which includes a modulator circuit 620 and a demodulator circuit 622. The demodulator circuit 622 is configured to recover information, such as commands or data, that the smart device 440 transmits and the antenna 601 receives and provides to the communications circuit 604. And the modulator circuit 620 is configured, effectively, to modulate the source NFC signal, via the antenna 601, with information, such as status or measurement data, generated by the controller circuit 626 for transmission to the smart device 440.

A memory circuit 624 includes one or both of volatile and non-volatile memory. For example, the non-volatile memory can be configured to store configuration data for configuring one or more of the circuits of the sensor patch 600, and a set of software instructions that, when executed by the controller circuit 626, cause the controller circuit, or one or more circuits under the control of the controller circuit, to execute a sensor measuring, reporting, and notifying cycle or cycles. The volatile memory of the memory circuit 624 can include registers and buffers configured for storing data received from the smart device 440 via the demodulator circuit 622 and data received by the controller circuit 626 for transmission to the smart device 440 via modulator circuit 620. In active power mode, the device can function as a data logger, using flash registers of memory circuit 624 to store data. In this embodiment, data from memory is read to the smart device 440 using the modulator 620 and NFC antenna 601.

Together the modulator and demodulator circuits 620 and 622 and any data-storage, decoding, and encoding circuitry that the modulator and demodulator include, the power circuit 602, and the memory circuit 624, form a wireless-communication circuit 618, which includes both the power-harvesting and information-exchange circuits of the sensor patch 600.

Wireless-communication circuit 618 has an integrated-circuit architecture. The integrated circuit includes the power circuit 602 and the communication circuit 604. The communication circuit 604 is configured to configure and to control the energy-harvester circuit 612 by loading configuration data from the memory circuit 624 into the power circuit 602, and also may obtain information from the power circuit 602 via link 616 (hence the bidirectional arrow/coupling between the energy-harvester circuit 612 and the communication circuit 604). For example, the wireless-communication circuit 618 is a single-chip component and is configured to operate with some level of independent functionality from the controller circuit 626. The circuit 618 is configured to generate an onboard voltage sufficient to wake up the controller circuit 626, for example, before the controller circuit can take a sensor measurement in passive NFC mode.

In an embodiment, the controller circuit 626 is configured to perform at least two functions. Sensor banks 631 and 632, which may be mounted on a flexible circuit support membrane, e.g., a substrate, 630 with the other circuits and components of the sensor patch 600, are in digital communication with the controller circuit 626. The sensors may be analog or digital, but the sensor package(s) for sensors S5, S6, S7 and S8, if these sensors are analog, can include an ADC to render these sensors compatible with a bus, such as an I²C bus 635.

To access sensor readouts for sensors S1, S2, S3, and S4, a serial bus 633 joined to a first bank 631 of these sensors may include a multiplexer 634 or multiple sensors joined to an I²C bus may include an I²C multiplexer switch on the bus. The controller circuit 626 is configured to receive sensor output and to process the sensor output for several purposes.

In a first executable step, the controller circuit 626 may process the sensor output according to programmable rules established by the manufacturer or programmable by the user, and transmit a command to a reporter component such as the RGB LED 638.

In a second executable step, the controller circuit 626 may include several data fields in a message and cause the data string to be transmitted via the modulator circuit 620 through antenna 601 to a proximate compatible smart device 440. The message includes a digital unique identifier UID that is assigned to the sensor patch 600. A timestamp may also be included.

From the smart device 440, the “notification” may be forwarded to LAN or WAN network components (not shown in FIG. 6) and to any remote workstation (not shown in FIG. 6) by a Bluetooth signal, by a cellular signal, by Wi-Fi, or by any wireless or wired system. The smart device 440, or a remote workstation in receipt of data may also present display tables and trendlines that show the latest sensor measurement in the context of past sensor measurements. By storing the data in a folder dedicated to the unique identifier UID of the sensor patch 600 and associating that folder with a particular target of the measurements (e.g., a patient, a sick child, a champagne bottle, a refrigerator, a package, a truck trailer, a runner in a race, a soldier, a blood bag, and so forth, without limitation) so that a permanent tracking history of the measurement is incorporated into the folder history, then that folder can be copied to other parties having an interest according to permissions and rules associated with the UID by the user or by a system administrator.

The controller circuit 626 also may command any optional reporter component 639 (such as a buzzer or vibrator) to call attention to the data. The reporter circuit 638, which can include one or more RGB LEDs, or the optional one or more reporter displays 639, can serve as an alarm if there is a critical sensor result, or can signal an “all clear” if the data is within expected limits.

As embodied in the sensor patch 600, sensor data is (A) sent as a wireless notification and (B) reported by some physical manifestation that is manifested in the sensor patch by reporter components 638 or 639, or can be an electrochromic patch that changes color or shape according to the sensor value as interpreted by the controller circuit 626. The wireless notification can include the UID of the sensor patch 600, but the direct display onboard the sensor patch need not display the UID because the reporter component is co-located with the patch sensor.

In variants of this architecture, the sensor patch 600 is fitted with an array of sensors, such as force sensors. The result is that by addressing each sensor by a particular address, such as on an I²C bus, and by timestamping each sensor output, a temporal and spatial map of deformations in the sensor patch 600 can be constructed. Such a force-sensing operation may have interest in clinical applications where heart pulse rate and pulse characteristics are studied at a peripheral artery. The sensor array also permits a user to place the sensor patch 600 on top of an artery, such as on the wrist, without exactly knowing where the radial artery is—the sensor patch 600 can be configured to detect the strongest signal and assess pulse rate accordingly. Similarly, the sensor patch 600 may be configured to assess peroneal, brachial, or carotid pulse without detailed palpation to determine the precise anatomy of the strongest signal. And by comparing pulse deformation along a series of sensors that follow the artery, the sensor patch 600 can integrate the size of the pulse wave. The integration, when placed in the context of a database of measurements made of patients in various stages of heart pathology or in treatment for heart pathology, can be used to make predictions about diagnosis, about prognosis, about the response to therapy, and can be used to alert the user (via the patch reporter) or a caregiver or administrator (via a patch notification) that something is amiss, that some new event is occurring, or that a series of measurements over time shows a steady improvement or a worsening condition. When confined with electrophysiology of the heart by use of an electrocardiogram (EKG), measurement of peripheral pulse volume can provide a convincing indication of cardiac ejection volume, a major predictor for morbidity and mortality in congestive heart failure. Thus, use of the sensor patch 600 in combination with a smart device for a daily home examination, when transmitted to an experienced clinician or to a cloud facility for making computerized evaluations, can result in improved outcomes by getting people to the emergency room when needed and by giving them the peace of mind to stay home when no intervention is called for.

Similarly, for athletes the sensor patch 600 can be configured to evaluate an athlete's response to training using cardiac output as a parameter. A sensor patch 600 with force sensors also may be adapted to monitor breathing rate and lung vital volume, factors of interest to pulmonologists and for athletes in training. An inexpensive disposable version of the sensor patch 600 that an athlete can use to record and to store key data after a workout offers not only improved individual training, but also can be used to compare training regimens across large groups, an application of big data made possible by easy access to physiological measurements.

The sensor patch 600 could facilitate further learning about real-time hematological indicia as well. For example, the science of blood oximetry is little studied in the general population. By assembling large cohorts of individuals and obtaining periodic measurements of blood oxygenation using a suitably configured sensor patch 600 with a simple photo-oximeter sensor (“pulse oximeter”), epidemiological studies of air quality, clinical studies of exposure to toxic pollutants, chronic occupational conditions, correlations with age and underlying conditions, and so forth, large volumes of data can be accumulated. Related big-data studies can be undertaken for diabetics and by looking at other blood markers, a broad range of human conditions in health and disease. The devices and systems disclosed here offer significant advances in telemedicine that may reduce costs of medical care while improving outcomes and providing safer environments and working conditions.

Still referring to FIG. 6, alternate embodiments of the sensor patch 600 are contemplated. For example, the sensor patch 600 can have an NFC/RFID tag architecture that includes the integrated controller circuit 626 and the integrated communications circuit including the modulator 620 and the demodulator 622, which interface with the NFC antenna 601. The communication circuit 604 and the controller circuit 626 can be on separate chips or on a same chip. Or, the one or more integrated circuits can be flexible chips such as supplied by American Semiconductor, Inc., of Boise, Id. Furthermore, features of embodiments described in conjunction with FIGS. 1-5B and 7A-37 may be applicable to the sensor patch 600 of FIG. 6.

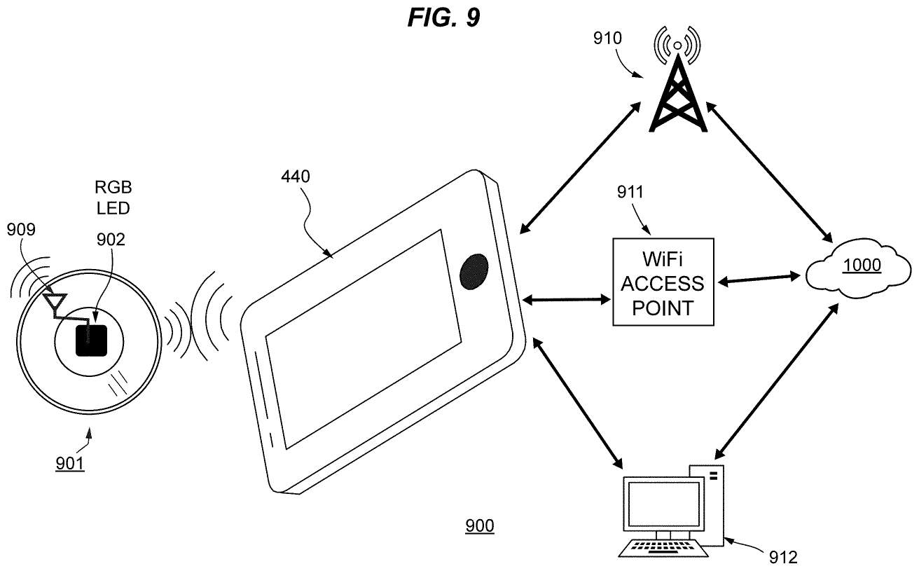

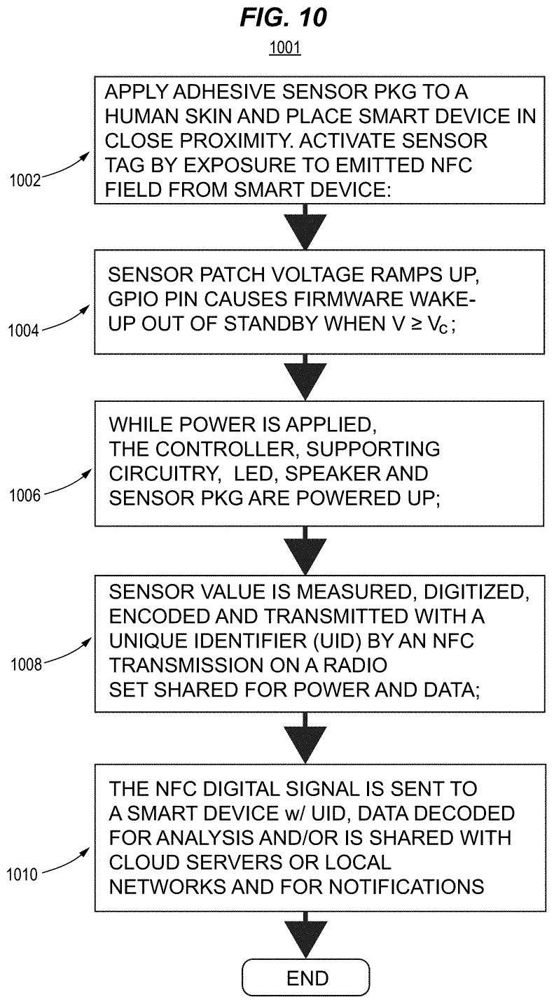

FIG. 7A is an exploded isometric view of a multi-layer sensor patch 700 according to an embodiment in which the sensor patch includes the circuitry of, and is otherwise similar to, the sensor patch 100 of FIG. 1. A middle layer 702 is a flexible circuit backing or substrate, for example a plastic that resists folding but bends easily. The conductive circuit-interconnection traces and pads are layered on the bottom face of the flexible substrate 702. A conductive glue is then used to attach the integrated-circuit (IC) chips and components to the conductive pads of the substrate 702. The sensor patch 700 includes a surface-mounted LED device 703 (e.g., an RGB LED) configured for bottom illumination (emission direction is “up”, see FIG. 7B) and a controller IC 704 (e.g., such as the controller circuits of FIGS. 1 and 6). A nested set of conductive loops forming an NFC antenna 705 are formed over a bottom (same side to which the components are mounted) of the substrate 702. Conductive test pads are also included on a same side of the substrate 702 as the conductive traces, and facilitate calibrating, loading of software and configuration data into, and testing of, the sensor patch 700 during manufacture and test. One or more other circuits and components may be omitted from FIG. 7A for clarity. Furthermore, in an embodiment, no “vias” through the substrate 702 are present because there are no circuits or components disposed on the other side (top side in FIG. 7A) of the substrate. Layer 704 includes a compliant film of a non-conductive adhesive. The adhesive may be selected to be biocompatible and adhere to an object, for example, to the skin of a human subject, or may be an adhesive such as used in packaging labels or industry. For example, the adhesive may be a nonconductive gel such as polyacrylate. The adhesive is applied over the circuitry and components. A removable release backing may be disposed over the adhesive layer 710 to prevent contamination of the adhesive layer before attachment of the sensor patch 700 to a subject or object, and may be peeled off of the adhesive layer just before attaching the sensor patch.

Alternatively, conductive traces and pads of the flexible circuit 702 are formed over a plastic flexible substrate and components are soldered to the conductive pads by remelt of solder balls to ensure good conductivity; a drop of dielectric epoxy flowed under each component may be used to strengthen the attachment of the component to the substrate. Alternatively, the components may be attached to the conductive pads with an electrically conductive glue. Overlayer 708 is a protective cushion of a translucent foam and bandage-like material that is configured to insulate, thermally, the circuit so as to prevent interference in temperature measurements when ambient temperature is low or high relative to the temperature of an object to which the sensor patch 700 is attached; the overlayer may also function to block water capillarity under the circuit backing.

Components may be electrically connected to the circuit leads using conventional wire bonding, solder bead-bump-on pad fusion, flip-chip fabrication technologies, or electrically conductive glue. For flip-chip fabrication, solder balls on the base of the chip are contacted with pads on the face of the circuit membrane, then re-melted (typically using hot-air reflow, or using a thermosonic bonding, see “reflow soldering”). The mounted chip is then “underfilled” using an electrically insulating adhesive of the desired stiffness. Thermal bonding can be a challenge if the two surfaces to be joined do not have matched thermal expansion. The underfill step prevents breaking the chip electrical connections under flex and prevents the die from being cracked and broken. Because these dies are on the order of 1 mm², the mechanical stress is manageable. A common-base die can be used to make “piggyback” or hybrid flip chips having the sensor built on a separate die and then stacked onto the flip chip before soldering. In this way, any one of multiple sensor types can be attached without the need to individualize the basic package.

Alternatively, clusters of chips can be wired together using connectors and pads lithographed in or on the flexible substrate. Flip chips also may be unique in that after soldering is completed, a layer of adhesive, such as an epoxy, is flowed under the chip and around the solder beads. This provides rigidity that can be localized specifically to the area of the chip and helps to insulate the connections while not restricting flexibility of the carrier or the bandage package.

Because even a non-conductive adhesive layer 704 may become conductive after exposure to moisture and salt, such as sweat on human skin, and because a conductive adhesive layer may “short out,” electrically, one or more conductive traces, such as the antenna loops, disposed over the substrate 702, the sensor patch 700 also can include an optional liquid-proof sealing layer 706 disposed between the substrate 702 and the adhesive layer 710. For example, the sealing layer 706 can be, or otherwise can include, a dielectric such as a potting material. Moreover, the non-conductive sealing layer 706 may allow the adhesive layer 710 to include an electrically conductive adhesive. The materials from which the adhesive layer 710 and optional sealing layer 706 are formed, and the thicknesses of these layers, render the adhesive layer and, if included, the sealing layer, thermally conductive so that a temperature sensor disposed over (e.g., on the bottom in FIG. 7A) the substrate 702 is thermally coupled to a surface for measurement of temperature.

The loops of the antenna 705 form a raised circumferential segment, and the antenna can be made (e.g., by etching) from a deposited layer of aluminum or other conductive metal (e.g., copper, an alloy) that has a bending modulus similar to the bending modulus of the substrate 702.

FIG. 7B is an exploded cross-sectional side view of the sensor patch 700 of FIG. 7A, according to an embodiment. The sensor patch 700 includes an RGB LED 703 and an IC control circuit 704, which can be similar to the RGB LED 124 and the controller circuit 114, respectively, of the sensor patch 100 of FIG. 1. A temperature sensor (not shown in FIG. 7B) can be integrated as part of the integrated circuit 704. The LED 703 is configured so that light exits the LED through the back (top in FIG. 7B) of the transparent substrate 702 and away from the adhesive layer 704. The color of the LED can have significance as described in printed instructions on the patch or on accompanying labelling. The gel capsule 707, also called a lens capsule, serves as a light pipe so that the LED color is displayed through the external insulative cover, according to an embodiment. Overlayer 708 is a protective cushion of a translucent foam and bandage-like material.

Because the controller circuit 704 is configured to actuate the LED 703 only after a temperature measurement is completed, and because the substrate 702 is less than a millimeter in thickness, the sensor patch 700 can omit a heat sink for the LED. The colored glow of the LED, while active, is readily visible through the transparent lens 707 in cover layer 708, and graphics may be applied to the exterior side (upper side of the insulative cover 708) of the substrate without interfering with the visibility of the LED.

As described above in conjunction with FIG. 7A, the adhesive layer 710 is disposed on the component side of the substrate 702, the component side being the side of the sensor patch 700 that contacts and adheres to skin or another surface while in use.

Referring to FIGS. 7A-7B, alternate embodiments of the sensor patch 700 are contemplated. For example, the substrate 702, the adhesive layer 710, and, if included, the sealing layer 706, can be formed from one or more “breathable” materials so that the sensor patch 700 can be attached to a human body (e.g., to human skin) for an extended period of time (e.g., hours, days) without irritating the skin or causing one or more other problems. Furthermore, in addition to the substrate 702 being flexible, one or more integrated circuits mounted to the substrate can be flexible integrated circuits such as available from American Semiconductor, Inc., of Boise, Id. In addition, features of embodiments described in conjunction with FIGS. 1-6 and 8-37 may be applicable to the sensor patch 700 of FIGS. 7A-7B.

The IC processor circuit (e.g., a microprocessor or microcontroller) 704, when used with this device, can be provided with software or firmware so as to enable display of pulse rate, blood pressure, temperature in Celsius or Fahrenheit, and so forth, using sensor outputs from an array of one or more sensors. For non-clinical applications, the display can summarize the historical temperature high and low, or any shock delivered to the system. In alternative embodiments, the pressure sensor can be substituted by or supplemented with an accelerometer in order to improve shock sensitivity and to permit better resolution of pulse beats in the human circulation, for example.

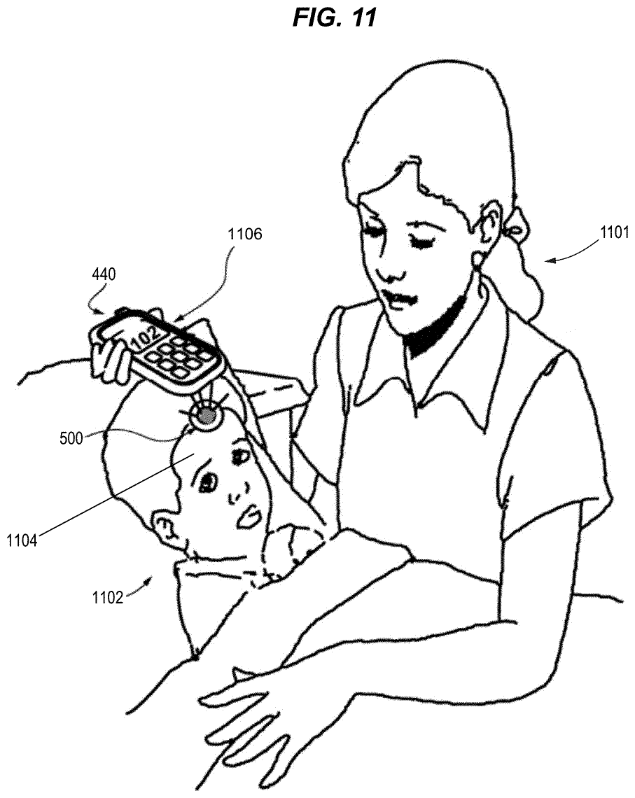

FIG. 8 is a plan view of a temperature sensor patch 800 having an alternate form factor and with a cover layer 841, according to an embodiment. The sensor patch 800 includes a flexible substrate 842, a thermistor 843, an RGB LED 844, a controller circuit 845 including a clock and cache memory, an antenna 846 having multiple loops or turns, and an NFC integrated circuit 847 having power (energy)-harvester, modulator, and demodulator circuits.

The antenna 846 includes a bridge 848 for coupling an outside end or node of the antenna to the NFC circuit 847; the inside end or node of the antenna is coupled to the NFC circuit directly. That is, the bridge 848 is a conductor that bridges the inner loops of the antenna 846 and that is electrically connected to the outer loop of the antenna at its outer node. The bridge 848 is electrically insulated from the inner loops of the antenna 846 by a dielectric or other non-electrically conductive material in a conventional manner. Alternative antennas that can be used in the sensor patch 800 instead of the loop antenna 846 include RFID, monopole, dipole, patch, microstrip, and fractal antennas such as proposed in U.S. Pat. Nos. 6,452,553 and 7,256,751, which are incorporated by reference, and at www.fractenna.com, the contents of which are incorporated by reference.

Still referring to FIG. 8, alternate embodiments of the sensor patch 800 are contemplated. For example, features of embodiments described in conjunction with FIGS. 1-7B and 9-37 may be applicable to the sensor patch of FIG. 8.

Referring to FIGS. 1-8, alternate embodiments of a sensor patch are contemplated. For example, a sensor patch can be part of a system for making sensor measurements using NFC wireless power transfer that powers both a local reporter display and a wireless remote notification. The sensor patch is typically passive but may contain charge-storage components (battery, capacitor) for extended use. The sensor patch receives power from an energy-harvesting circuit that generates a power signal in response to the sensor patch being exposed to an NFC field emitted by a smart device (e.g., a smartphone 440) that is placed in close proximity to the sensor patch and actuated. Generally, the smart device is programmable and operates with an installable application and a graphical user interface (GUI). Different programming and GUIs may be used for different measurements, making the combination of sensor patch and smart device a flexible and robust system for sensing, monitoring, and reporting sensed quantities and conditions.

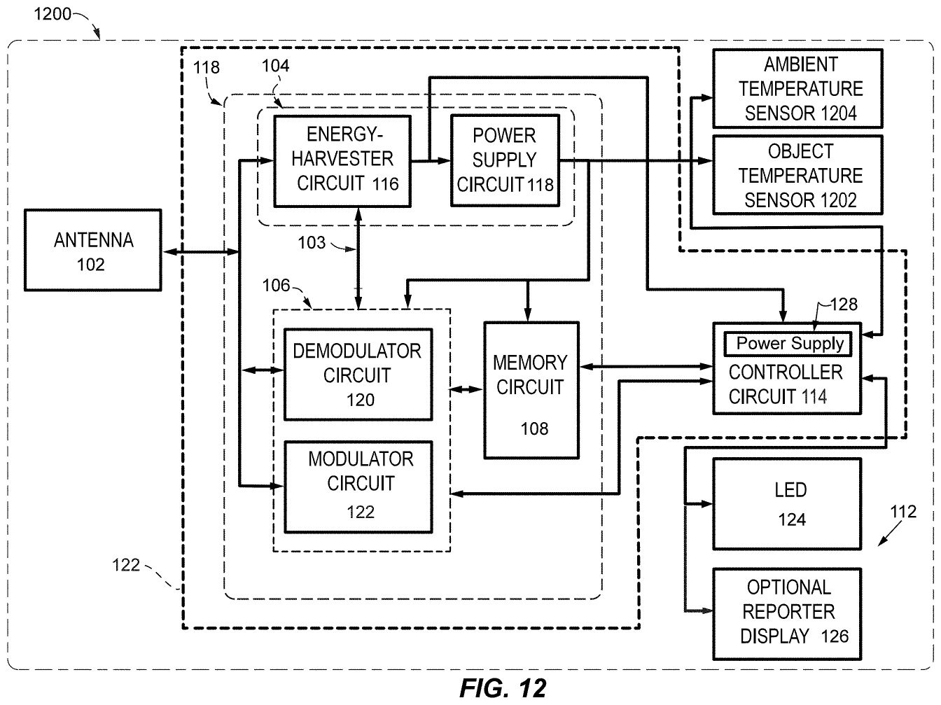

FIG. 9 is a diagram of a system 900 configured to make sensor measurements and to network sensor data to a local area network (LAN) 912, wide area network (WAN) via Wi-Fi 911, one or more cellular 910 networks, or a sub-combination or combination of any of the preceding networks and other networks, according to an embodiment. Micro-networks, such as Manet, and LAN networks, such as Bluetooth, may also be used to connect the smart device 440 to other computing components of the system. As will be described in more detail below, the sensor patches are not limited to NFC-modulation for data exchange and can include a variety of low-energy actively powered antennas such a BT antenna 909.

The smart device 440 (e.g., a smart phone) is configured to communicate with, and to power, a sensor patch 901 via electrical, magnetic, or inductive coupling between antennas of the smart device and the sensor patch, for example according to an NFC protocol or an RFID protocol. Operational distance between the smart device 440 and the sensor patch 901 can be, for example, in a range of approximately 30 cm or less, or 20 cm or less, or 1 cm or less.

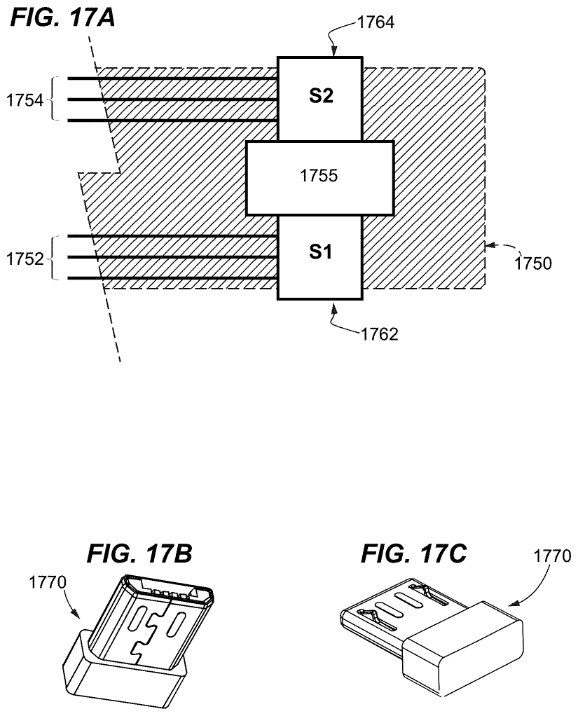

The sensor patch 901 can be configured to provide an indication of a measured quantity (e.g., human temperature within or not within normal range). The sensor patch 901 can be configured to send a value of the sensed quantity to the smart device 440, which can be configured to communicate with a remote device via a Wi-Fi access point 911, a cell tower, or cellular network 1410, with another device (e.g., a computer or workstation 912), or with an administrative host (not shown in FIG. 14). All of these intermediate devices can be configured to communicate with a “cloud host” 1000 or with a remote device via the Internet. The data can be analyzed by, e.g., a medical professional so as to enable applications for telemedicine. The data can also be programmed and used in a household or business to enable better monitoring and notifications of sensor conditions that require attention. Furthermore, the sensor patch 901 can be considered an internet-of-things (IoT) device.

The sensor patch 901 and smart device 440 are configured to work together to share data with the IoT on the cloud host 1000 or on local terminals. One or more network links and portals may be used to forward data from the sensor patch 901 to a higher-level data-processing system. Once data is captured by the smart device 440, it may be stored or manipulated locally, including for display and for making rules-based notifications that are associated with thresholds or states of sensor output.

Data shared with the larger system networks can be stored and archived, displayed as needed, and notifications can be sent to remote devices for action or for tracking. Each sensor patch 901 in the system 900 (the system also can be configured to include multiple sensor patches) is provided with a unique identifier that is sent digitally to a smart device 440 in response to a measurement being made and the user has the option to associate that digital identifier with a particular patient, child, or inanimate object for which sensor data is being collected. The program on the smart device 440 can provide user profiles so that the sensor-patch UID can be attached to a particular patient or object. Data that is identified by UID and timestamp can be aggregated over time and trends plotted or tracked.

Notifications can be issued according to the trends in the data and in compliance with rules and permissions established by the end user or a system administrator, for example an administrative host associated with an IP address in cloud host 1000.

For example, a software application can configure any suitable smart device 440 as a thermometer that can read a temperature from the sensor patch. The smart device 440 includes a controller circuit (e.g., a microprocessor or microcontroller) with NFC transmission capability and is in communication with an administrative host server on the cloud host 1000. The local smart device 440 or a cloud-hosted administrative server may be configured to provide temperature charting, and remote notifications as needed, and to display enhanced plots or annotations either on the small screen of the smart device or on a desktop monitor such as at a nursing station.

The charting and display functions are part of an electronic medical record and are automatic once the measurement cycle is activated by bringing the smart device 440 into NFC proximity with the sensor patch 901. During measurement, data exchange can be bidirectional (e.g., time-division multiplexed or frequency-division multiplexed) as described, for example, so that sensor parameters can be updated while sensor data is being collected or sequentially. The smart device 440 also can be adapted to make other sensor measurements by providing sensor patches 901 with suitable sensors and a suitable software application installable on the smart device.

Still referring to FIG. 9, alternate embodiments of the system 900 are contemplated. For example, features of embodiments described in conjunction with FIGS. 1-8 and 15-37 may be applicable to the system 900 of FIG. 9.

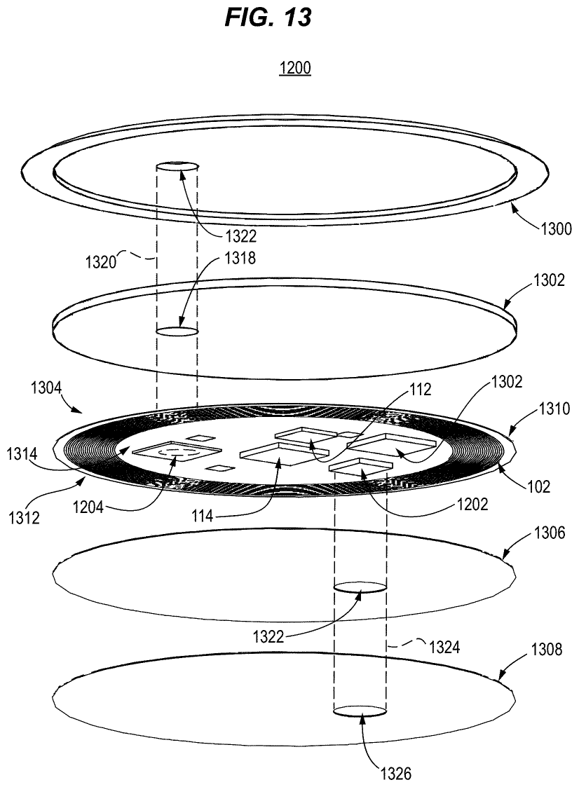

FIG. 10 is a flow diagram of a method 1001 for making a sensor measurement using a sensor patch such as described above in conjunction with FIGS. 1-9, according to an embodiment. The nature of the sensor patch is agnostic and temperature sensors, pressure sensors, and accelerometers have been illustrated as exemplary sensors, while not limiting the applicability to other sensors wired in series or in parallel to the controller circuit or operated as multiplexed sensor modules.

At a first step 1002, a user applies the adhesive sensor patch to a surface to be monitored and places a smart device that has been programmed for operating with the sensor patch in close proximity to the sensor patch. The sensor patch activates in response to a source power-and-information signal from a nearby (e.g., within a range of one foot or less) smart device such as a smart phone; the active sensor patch harvests energy from the source signal.

At a step 1004, a supply voltage that the sensor patch generates in response to the harvested energy ramps up, and, at a step 1006, in response to the supply voltage equaling or exceeding a threshold voltage, the controller circuit wakes up and may cause one or more other components (e.g., sensor, speaker) of the sensor patch to power up, or otherwise to wake up, out of a standby or sleep mode. For example, a logic level on a general-purpose input-output (GPIO) pin of a power circuit that includes an energy-harvester circuit transitions to an enable state in response to the supply voltage equaling or exceeding a threshold voltage. Or the controller circuit otherwise can be configured to wake up in response to the supply voltage equaling or exceeding a threshold voltage.

At a step 1008, the sensor patch initiates a measurement, for example, in response to a command from the smart device, and a sensor of the sensor patch generates a sense signal that is digital, that an ADC converts into a digital sense signal, or that the sensor patch digitizes by pulse counting if the sensor is, for example, a capacitive sensor. The sensor patch then encodes the digital signal with a unique identifier (UID) and transmits the encoded digital signal to the smart device by modulating a carrier wave of the source signal via the sensor-patch antenna. For example, the smart device modulates the carrier wave at a center frequency of 13.6 GHz with an information message, the sensor patch extracts the information message by demodulating the modulated carrier wave and harvests power from the carrier wave, and then the sensor patch modulates the carrier wave with a patch-information message such that unidirectional or bidirectional communication between the smart device and the sensor patch, and power harvesting by the sensor patch, can occur simultaneously. Alternately, communications can be time-division multiplexed so that at any one time the information is flowing in only one direction between the smart device and the sensor patch.

At a step 1010, the sensor patch sends a sensor-patch information signal to the smart device, and the sensor-patch information signal includes a UID of the sensor patch, and the smart device demodulates, decodes, and recovers the sensor-patch information from the sensor-patch information signal, and processes the recovered information. For example, the smart device may send or share the recovered information to or with local computing resources, remote system resources, and cloud servers or any local radio links that are authorized to receive the sensor-patch information. Following analysis at any level in the system, notifications and commands may be sent to one or more of the smart device and the sensor patch. The smart device also may archive the sensor information along with a timestamp and any other relevant information derived from the UID. A profile that has been set up for a particular sensor patch and associated with a particular patient or object is updated with current information. Security and other properties in the profile are typically set by the user or by a system administrator.

Next, the sensor patch shuts down when power (e.g., the source signal generated by the smart device) is withdrawn or when the measurement is completed. In some instances, onboard power will be stored so that an extended function such as a speaker or a series of sensors can be operated.

For passive operation, during the initial step 1002, an operator brings the smart device into close proximity (e.g., within one foot) with the sensor patch and initiates a program or “application” on the smart device. The program causes the device, while executing instructions of the program, to emit an NFC radio signal, for example, at 13.56 MHz with an amplitude sufficient to power the sensor patch. The controller circuit of the sensor patch wakes up when the supply voltage that a power circuit onboard the sensor patch generates equals or exceeds a defined threshold voltage.

The controller circuit onboard the sensor patch, as configured by configuration data (e.g., firmware) or while executing software, causes a temperature sensor to be powered and enabled so that the sensor can measure a temperature.

In step 1008, a sense signal generated by the sensor is converted into a digital signal and a return transmission of the sensed temperature is made from the sensor patch NFC radioset to the smart device. The transmission is generally encoded by modulating the carrier frequency of the source signal generated by the smart device. Once the sensed temperature value is received by the smart device, the smart device causes the data to be distributed through a local or wide area network where other programming may be used to archive and to display the sensor output in context of previous measurements and to send out an alarm if the temperature is at variance with expected normal results.

In addition, the skin-adhesive sensor patch is provided with an LED or LEDs and is configured so that a visual indication of the temperature as high or low accompanies a measurement. The LED may be a 3-pin RGB LED for multicolor use or a pair of LEDs, one red and one green, can be provided so that the method permits the user to rapidly determine whether the skin temperature is elevated by applying NFC energy, typically by bringing a smart device into proximity of the sensor patch. Other visual indicators may be used. Alternatively, a vibration or a sound can serve as an indicator of the temperature at the bedside, without reliance on an indirect readout from the smart device. These and other features can be controlled by firmware that configures one or more of the circuits embedded in the skin-adhesive package of the sensor patch and by software installed on the smart device, such software as is typically termed an “app”.

The method also can include a local display that is indicative of the sensor output, either a parametric output or a qualitative output. The visual indicator may blink, change output frequency, color, and the sensor patch may also send data to memory. The sensor patch need not transmit data but can instead operate to display the temperature only visually on a visual display or can both visually display the sensed temperate and transmit the temperature to the smart device for display or other action.

For example a user, the sensor-patch manufacturer, or other provider of the sensor patch, can set a high temperature threshold and a low temperature threshold that defines a fever (or chills) and a sensor signal that meets the required criteria will result in a display identifiable as significant, such as a RED color LED to indicate an abnormal high temperature or a BLUE color LED to indicate a chill. A GREEN color LED can indicate a temperature within a normal range, as a practical illustration. The smart device also can look up previous temperatures associated with a profile and analyze the data for trends. The smart device, if a trend is detected, can issue, to the sensor patch, a command by which a unique display is initiated. For example, if a fever is continuing to go up, the controller circuit of the sensor patch can cause the LED to flash a rapid series of pulses, and if the fever has broken and is starting down, the controller circuit can cause the LED to flash RED and GREEN in alternation. An indicator, such as an RGB LED, on the sensor patch, while typically having four leads and hence more controller complexity, has an advantage that there is an immediate, real time, point-of-care notification that does not require viewing the smart device or pressing a button to have an understanding of the measurement result.

A user, such as a caregiver or subject under care, can consult the smart device's display for more detail, for plotting functions, and for later access, but is not needed to get the information that is immediately presented by the sensor-patch LED or by some other reporter component such as a buzzer, beeper, vibrator, or other direct sensory input. By programming the smart device to issue commands to the sensor patch, or by supplying firmware or software with the sensor patch, a method is achieved by which a user can receive a local and a remote notification, and the remote notification can be shared with others or with a remote network. The inclusion of a local reporter structure, as exemplified by a variable color LED, is an advance in the art and provides a solution to a longstanding problem of rapidly assessing temperature (or some other sensor output) by an easy-to-understand symbolic language (color, pulsation, tone, and so forth) without the need to squint at a thermometer or angle an LCD screen so that the result is visible with backlighting.

And all the features described here can be achieved with a battery-less sensor patch that is disposable. By incorporating all the electronics, the antenna, and the IC components on a single layer, a simple device results that is inexpensive to make and simple to use. The ability to command the sensor patch according to the kind of measurement being made and to add programming so that the UID can be associated with a profile unique to an individual or an object, a robust and powerful means for accessing sensor data and sharing it with the IoT is achieved.

Still referring to FIG. 10, alternate embodiments of the described method are contemplated. For example, features of embodiments described in conjunction with FIGS. 1-9 and 11-37 may be applicable to the method described in conjunction with FIG. 10.

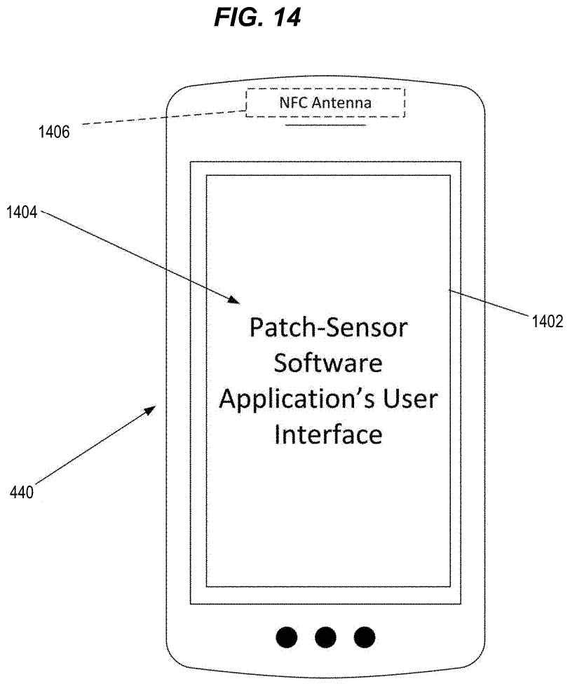

FIG. 11 is a view of a caregiver 1101 taking a temperature of a child 1102 using the sensor patch 500 and the smartphone 440, according to an embodiment. FIG. 11 illustrates context of use of a sensor patch 500 configured to sense temperature of an object to which the sensor patch is attached, according to an embodiment. For example, the sensor patch 500 may be the same as, or similar to, any one or more of the sensor patches described above in conjunction with FIGS. 1-9 and the figures below. In this example, the sensor patch 500 has been applied to the skin of a forehead 1104 of a child 1102, and a caregiver, such as a parent 1101, is using a smartphone 440 to take a temperature reading (shown here as 102° F. in a display window 1106 of the smartphone). The temperature reading may be analyzed by specialized tools on the smart phone, optionally with added tools available through a WAN (such as for telemedicine, for example, where the data is shared with a clinician). However, the temperature will also be displayed to the parent in a directly accessible color that communicates the significance of the sensor result. Normal and warning indications are reported immediately at the point of care (e.g., flash LED green if temp in range, flash LED red if temp too high, flash LED blue if temp too low). After transmission to higher system resources, any result of further analysis may be used to select or to modify the nature of the local display, or there can be rules based decision making directly in the sensor patch or directly in the smart device that determines how the report will be displayed. By permitting users to program their own rules for interpreting temperature measurements (e.g., a respective rule for interpreting temperature for each of multiple temperature-measurement locations on a body, such as forehead or armpit), a powerful apparatus for home, hospital, and other use is achieved. The devices may be stored in the same way that bandages are stored and have a shelf life that can be measured in years. Software updates to the software application installed on the smartphone 440 are handled by the smartphone and require no special reconditioning of the sensor patches. And software and firmware updates to the sensor patch 500 also can be accomplished via the smartphone 440.

For the temperature of a human body, the temperature sensor onboard the sensor patch 500 can include a thermistor such as the Murata NCP18XH103D03RB, which has a resistance of 10 kΩ and linearity of 3380K±0.7% from 25 to 50° C., and offers low power consumption (e.g., low enough to be part of a device that is powered by an NFC signal) and suitable accuracy in a clinical range.

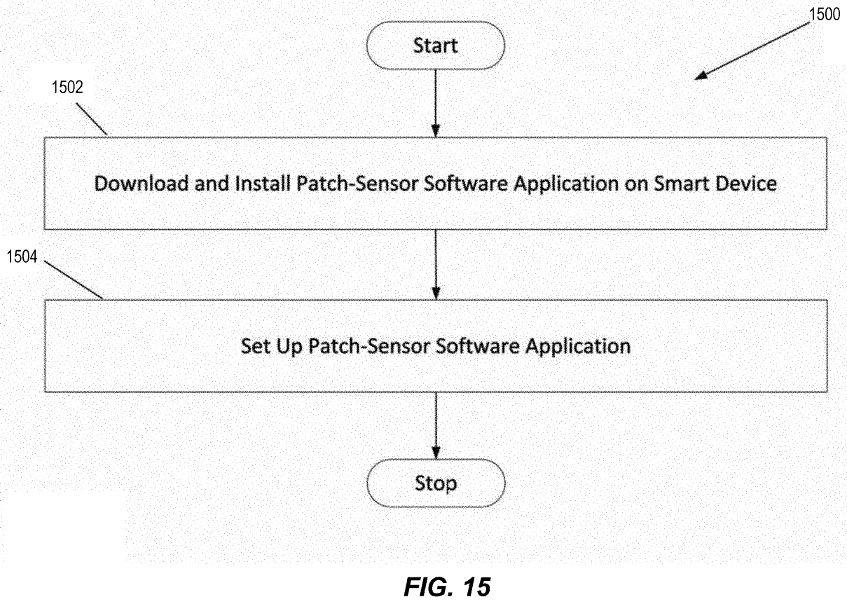

FIG. 12 is a circuit diagram of a sensor patch 1200, according to an alternate embodiment in which the sensor patch 1200 is similar in circuit topology, structure, and operation to the sensor patch 100 of FIG. 1, and where like numbers reference components common to FIGS. 1 and 12. Differences between the sensor patch 1200 and the sensor patch 100 include that in the sensor patch 1200, the sensor(s) 110 (FIG. 1) include, or are replaced with, an object-temperature sensor 1202 and an ambient-temperature sensor 1204, and that the object- and ambient-temperature sensors, the RGB LED 124, and the reporter display 126 each receive a regulated supply voltage directly from the power-supply circuit 118 instead of, or in addition to, receiving a regulated supply voltage from the controller circuit power supply 128.

The antenna 102, power circuit 104, communication circuit 106, memory circuit 108, reporter circuit 112, and controller circuit 114 each are the same as, or similar to, the corresponding component as described above in conjunction with FIG. 1.

The object-temperature sensor 1202 is configured to sense the temperature of an object (e.g., human forehead, bottle of baby's milk) to which the sensor patch 1200 is attached, to generate a sense signal that is related to a value of the sensed temperature, and to provide the sense signal to the controller circuit 114. For example, the object-temperature sensor 1202 can include the analog, thermistor-based temperature sensor 210 of FIG. 2A, or can include a digital temperature sensor such as part STTS22H from STMicroelectronics, Inc. of Carrollton, Tex. An analog object-temperature sensor can be calibrated during manufacture and test of the sensor patch 1200 in a manner similar to that described above in conjunction with the sensor patch 100 of FIG. 1, and a digital object-temperature sensor can be calibrated, if needed, in any suitable manner.

The ambient-temperature sensor 1204 is configured to sense the ambient temperature, i.e., the temperature of the environment, or substance (e.g., air) in which the sensor patch 1200 is immersed. The environmental temperature may cause an error in the temperature of the object as sensed by the object-temperature sensor 1202, and, therefore, the controller circuit 114, or a smart device (not shown in FIG. 12) in communication with the sensor patch 1200, can correct the error in response to the ambient temperature sensed by the ambient-temperature sensor or cause an alert to be displayed if the required correction is out of range. For example, if a subject is lying out in the sun on a hot day, a region of the subject's skin (e.g., the skin over the subject's forehead) may be significantly warmer than the subject's actual body temperature; or, the hot sun may “heat up” the sensor patch 1200, and, therefore, may heat up the object-temperature sensor 1202, such that the object-temperature sensor senses a temperature that is higher than the subject's actual body temperature. Or, if a subject is skiing on a cold, cloudy day, a region of the subject's skin (e.g., the skin over the subject's forehead) may be cooler than the subject's actual body temperature; or, the cold air may “cool down” the sensor patch 1200, and, therefore, may cool down the object-temperature sensor 1202, such that the object-temperature sensor senses a temperature that is lower than the subject's actual body temperature. To correct an error in the temperature sensed by the object-temperature sensor 1202, one can develop an algorithm in which the resulting (accurate) temperature of the object is a function of the object temperature sensed by the object-temperature sensor 1202 and the ambient temperature sensed by the ambient-temperature sensor 1204. Coefficients and constants of one or more equations representing the algorithm can be stored in the memory circuit 108 or in a memory of a smart device in communication with the sensor patch 1200, and the controller 114, or the smart device, can solve the one or more equations using the sensed values of the ambient and object temperatures to obtain an actual temperature of the object to which the patch sensor 1200 is attached.

Furthermore, the ambient-temperature sensor 1204 also can include the analog, thermistor-based temperature sensor 210 of FIG. 2A. An analog ambient-temperature sensor can be calibrated during manufacture and test of the sensor patch 1200 in a manner similar to that described above in conjunction with the sensor patch 100 of FIG. 1, and a digital ambient-temperature sensor can be calibrated, if needed, in any suitable manner.

Still referring to FIG. 12, alternate embodiments of the described method are contemplated. For example, instead of or in addition to being configured for operating according to an NFC communication and power-transfer protocol, the sensor patch 1900 can be configured for operating according to an RFID communication and power-transfer protocol. Furthermore, features of embodiments described in conjunction with FIGS. 1-11 and 13-37 may be applicable to the sensor patch 1200 of FIG. 12.

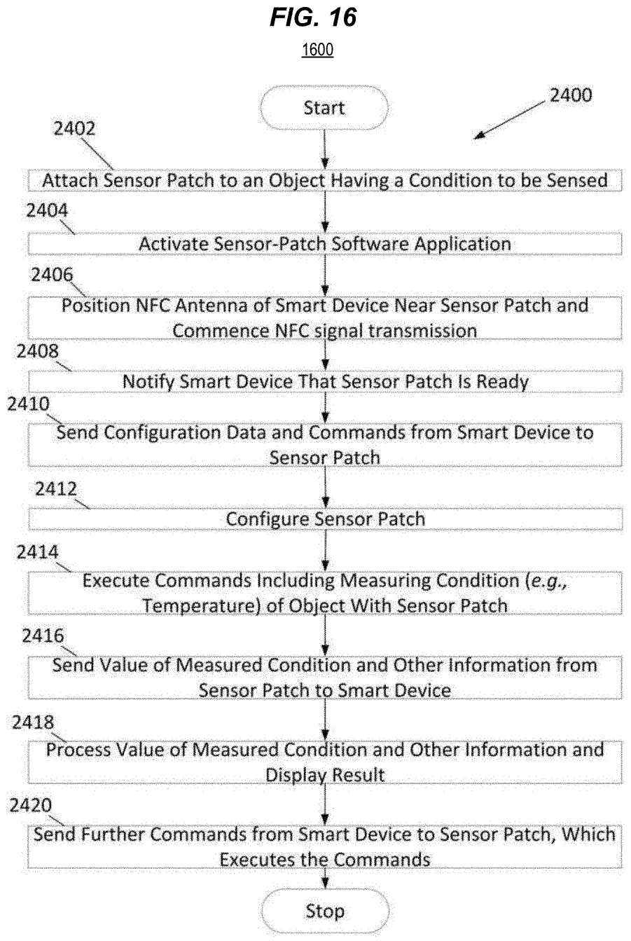

FIG. 13 is an exploded view of the patch sensor 1200 of FIG. 12, according to an embodiment. In addition to the components described above in conjunction with FIG. 12, the patch sensor 1200 includes a cover 1300, an insulator layer 1302, a flexible and transparent substrate 1310 with circuitry 1304, a protective sealing layer 1306, and an adhesive layer 1308.

Referring to FIG. 13, the antenna 102 is looped around a perimeter of substrate 1310, and disposed over an underside surface 1312, of the substrate 1310, and conductive circuit-interconnection traces and conductive component-attachment pads are disposed over the surface within an inner region 1314 of the substrate bounded by the antenna.

Leads of an integrated circuit 1316, which includes the power circuit 104, communication circuit 106, and memory circuit 108 of FIG. 12, are connected to respective ones of the conductive pads within the region 1314 with a conductive glue. The connection of the leads can serve to mount the integrated circuit 1316 to the substrate 1304, although the package of the integrated circuit may be attached to the surface 1312 of the substrate 1304 with a nonconductive adhesive such as epoxy.

Leads of the reporter circuit 112, controller circuit 114, object-temperature sensor 1202, and ambient-temperature sensor 1204 also are connected, with a conductive glue, to respective pads formed on the surface 2012 of the substrate 2004 within the region 2014. The connection of the leads can serve to mount these components to the substrate 1304, although the packages of these components may be attached to the surface 1314 of the substrate 1304 with a nonconductive adhesive such as epoxy.

The insulator layer 1302 is configured to provide a thermal barrier between the object-temperature sensor 1202 and the environment (e.g., the atmosphere) in which the sensor patch 1200 is immersed, can be made from any suitable thermally insulating material such as foam, and includes a portion 1322 of an ambient-temperature-sensor “heat pipe” 1324 that is aligned with the ambient-temperature sensor 1204. The heat pipe 1320 is a conduit that is configured to couple, thermally, the ambient-temperature sensor 1204 to the environment (e.g., the atmosphere) in which the sensor patch 1200 is immersed so that the ambient-temperature sensor can sense the ambient temperature, e.g., the temperature of the environment, more accurately as compared to a sensor patch without the heat pipe. The portion 1318 of the heat pipe 1320 is an opening formed in the layer 1302 and aligned with the ambient-temperature sensor 1204.

The cover 1300 is configured to protect the insulator layer 1302, can be configured to provide a further thermal barrier between the object-temperature sensor 1202 and the environment (e.g., the atmosphere) in which the sensor patch 1200 is immersed, can be made from any suitable material such as foam with a liquid-impervious (e.g., plastic) outer “skin,” and includes a portion 1322 of the ambient-temperature-sensor heat pipe 1320. The portion 1322 of the heat pipe 1320 is an opening formed in the cover 1300 and is aligned with the portion 1318 formed in the layer 1302 and with the ambient-temperature sensor 1204.

The protective sealing layer 1306 is configured to shield the circuit components and conductive traces disposed on the surface 1314 of the substrate 1304 from contaminants, such as from a conductive liquid such as water, and from problems caused by such contaminants, such as short circuiting. For example, the adhesive layer 1308 may absorb a conductive liquid, such as sweat from the skin of a subject to which the sensor patch 1200 is attached and condensation from another object (e.g., chilled bottle), and this absorbed liquid may migrate to the substrate surface 1312 and cause one or more problems such as the aforementioned short circuiting. The layer 1306 can be made from any suitable liquid-impervious material; for example, the layer can be a thin coating of sealant epoxy that is applied over the components and conductive traces disposed on the substrate surface 1312, and over exposed portions of the substrate surface, before application of an adhesive to form the adhesive layer 1308. Furthermore, the layer 1306 includes a portion 1322 of an object-temperature-sensor heat pipe 1324 that is aligned with the object-temperature sensor 1202. The heat pipe 1324 can be similar to the heat pipe 1320; for example, the heat pipe 1324 is a conduit that is configured to couple, thermally, the object-temperature sensor 1202 to the object (e.g., skin region of a human subject) to which the sensor patch 1200 is attached so that the object-temperature sensor can sense the object temperature, e.g., the temperature of a human subject's forehead, more accurately as compared to a sensor patch without the heat pipe 1324. The portion 1322 of the heat pipe 1324 is an opening formed in the layer 1306 and aligned with the object-temperature sensor 1202.

The adhesive layer 1308 is configured to attach the sensor patch 1200 to an object, can be made from any suitable material such as a non-electrically conducting adhesive, and includes a portion 1326 of the object-temperature-sensor heat pipe 1324. The portion 1326 of the heat pipe 1324 is an opening formed in the cover layer 1308 and aligned with the portion 1322 formed in the layer 2006 and with the object-temperature sensor 1202. Alternatively, due to the presence of the protective sealing layer 1306, the layer 1308 may include an electrically conducting adhesive, or an adhesive that may become electrically conductive due to, e.g., absorption of water such as found in sweat.

In one embodiment, the sensor patch 1200 operates with a smart device 440 that transmits and receives data from the sensor patch. Many of the calculations and notifications to a user are made by the smart device. Software installed on the smart device is designed to control and operate the sensor patch.

FIG. 14 is a plan view of a smartphone 440 on which is installed a sensor-patch software application, or “app,” that, when executed by the smart phone, allows a user of the smartphone to use a sensor patch such as the sensor patch 1200 of FIG. 12, according to an embodiment. The smartphone 440 includes a display screen 1402 on which the smartphone is configured to display a sensor-patch user interface 1404 in response to executing the software application. Furthermore, the smartphone 440 includes an NFC antenna 1406.

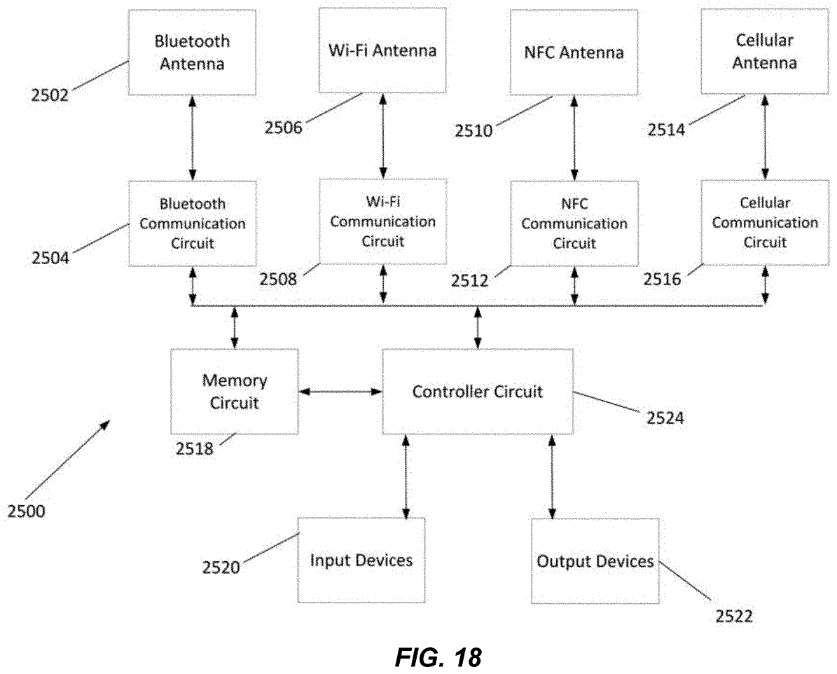

FIG. 15 is a flow diagram 1500 of a method for downloading, installing, and setting up a sensor-patch software application on the smartphone 440 of FIG. 14, according to an embodiment.

Referring to FIGS. 14-15, described is a method for downloading, installing, and setting up a sensor-patch software application on the smartphone 2100, according to an embodiment.

At a step 1502, a user first causes the smartphone 440 to download, e.g., from the internet, and install, in a conventional manner, the sensor-patch software application. For example, the smartphone 440 can download the sensor-patch software application from a website run by the sensor-patch provider, Google Play®, or the Apple Store®.

Next, at a step 1504, the user sets up the software app via the sensor-patch user interface 2104. For example, the smartphone 440 runs a setup routine of the software app, and in response to the setup routine, generates and displays, on the display 1402, a menu or questionnaire that requests the user to enter one or more pieces of information, such as the user's name and the type of the smart phone. A reason for the latter request is because each make and model of the smartphone 440 may include an NFC antenna in a respective different relative location, and the software app, during at least a temperature-sensing mode, may cause the smartphone to display the location of the smart phone's NFC antenna to a user so that the user can position the smart phone's NFC antenna sufficiently proximate to the sensor patch to activate the sensor. For example, the smartphone 440 may store a look-up table (LUT) that relates phone make and model to NFC antenna location, or the smart phone, as part of the setup protocol, may obtain the NFC antenna location via a database or other location accessible via the internet. Moreover, the menu may “walk” the user through setting up an internet or “cloud” account for storing information such as temperature trends and other data for one or more subjects.

After installation, the smartphone 440 may run the sensor-patch software application in the background while not being used or otherwise activated, or the smartphone may render the sensor-patch software application inactive until started by a user or otherwise.

Still referring to FIGS. 14-15, alternate embodiments of the method represented by the flow diagram 1500 are contemplated. For example, the smartphone 440 may sense at least some of the setup information (e.g., the make, model, and serial number of the smart phone) automatically. Furthermore, features of embodiments described in conjunction with FIGS. 1-13 and 16-37 may be applicable to the smartphone 440 of FIG. 14 and the method represented by the flow diagram 1500 of FIG. 15.

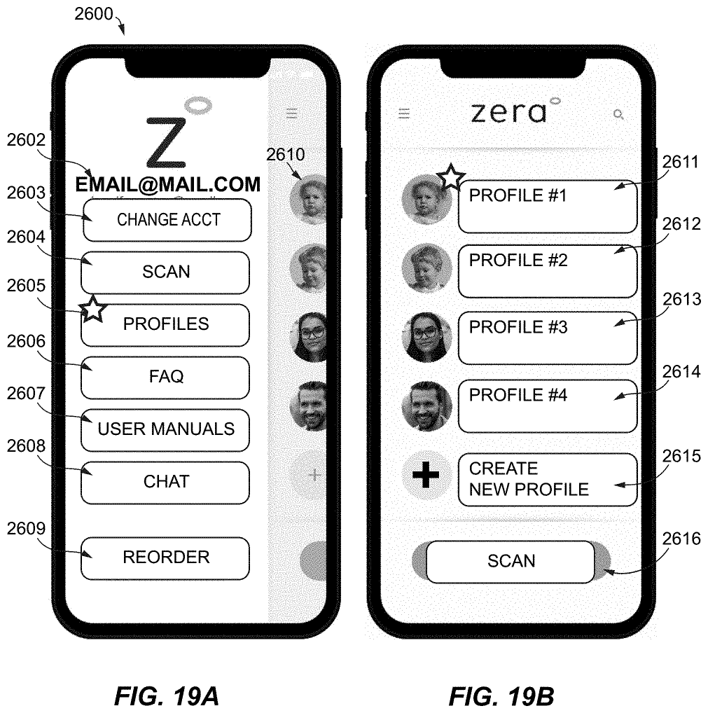

FIG. 16 is a flow diagram 1600 of a method for taking a temperature of the child 1102 of FIG. 11 using the sensor patch 1200 (FIGS. 12-13) and the smartphone 440 (FIGS. 14 and 15), according to an embodiment.

At a step 2402 of the flow diagram 2400, the caregiver 1101 attaches the sensor patch 1200 to a region, here a forehead 1104, of the child 1102. For example, the caregiver peels, from the adhesive layer 1308, a plastic release backing. The plastic protector can be similar to the plastic protector that one peels from the “sticky” part of an adhesive bandage before applying the bandage), places the sensor patch 1200 on the skin of the child 1102 such that the adhesive layer is against the skin, and presses the sensor patch against the child's skin (here the skin of the forehead 1104) such that the adhesive layer affixes the sensor patch to the child's forehead. Before placing the sensor patch 1200 on the forehead 1104 of the child 1102, the caregiver 1101 may wipe, or otherwise clean, the child's forehead to remove, from the child's skin, contaminants such as dirt, body oil, and sweat that may reduce the adhesion between the child's skin and the adhesive layer 1308 as compared to the adhesion with fewer contaminants present.

Next, at a step 2404 of the flow diagram 1600, the caregiver 1101 activates the sensor-patch software application installed on the smartphone 440. For example, the caregiver 1101 navigates to a menu (e.g., by “swiping” her finger) that the smartphone 440 displays on the display 2102 and that includes an icon corresponding to the sensor-patch software application, and touches with her finger, or otherwise selects, the icon. In response to the caregiver 1101 selecting the icon, the smartphone 440 loads the application into working (typically volatile) memory of the smart phone, and runs the application by fetching, from the working memory, and executing, one or more instructions that the application includes. The running application initially causes the smartphone 440 to generate, on the display 2102, a user interface 1404, which provides information to the caregiver 1101. For example, the user interface 1404 shows the caregiver 1101 the relative location of the smart phone's NFC antenna 1406, instructs the caregiver to position the smartphone 440 near the sensor patch 1200 and includes an illustration as to how the caregiver should hold the smartphone relative to the sensor patch. Further in example, the user interface 1404 shows the caregiver 1101 a suitable distance, or suitable range of distances, between the smartphone and the sensor patch, and shows a suitable position, or a suitable range of positions, of the smartphone relative to the sensor patch, so as to facilitate suitable transferring of power and information from the smartphone to the sensor patch, and to facilitate suitable transferring of information from the sensor patch to the smart phone. Yet further in example, the user interface 1404 may indicate as a suitable distance and position holding the top (the location of the NFC antenna 1406) of the smartphone 440 no more than approximately six inches from the sensor patch 1200.

Then, at a step 2406, the caregiver 1101 positions the smartphone 440 relative to the sensor patch 1200 as indicated by the user interface 1404 and causes the smartphone to begin transmitting an NFC source signal that includes a carrier, or power, signal. For example, to cause the smartphone 440 to begin transmitting the NFC source signal, the caregiver 1101 touches a “start” button of the user interface 1404 or issues a corresponding voice command.

Next, at a step 2408, in response to receiving the NFC source signal from the smartphone 440, the sensor patch 1200 sends, to the smart phone, a “ready” or other acknowledgement signal (e.g., a handshake signal) to indicate that the sensor patch is powered and is ready to receive configuration data and one or more commands from the smart phone. For example, in response to receiving the source signal, the antenna 102 generates a corresponding receive signal. And in response to the receive signal, the energy harvester circuit 116 automatically begins generating a raw power signal, the power-supply circuit 128 automatically begins generating a regulated supply voltage in response to the raw power signal, and a POR circuit (not shown in FIG. 16) onboard the controller circuit 114, or elsewhere onboard the sensor patch 1200, automatically generates a POR signal having an enable level in response to the regulated supply voltage attaining a threshold level. Then, in response to the enable level of the POR signal, the controller circuit 114 generates an acknowledgement message and stores the message in the memory 108 or provides the message directly to the communication circuit 106. The modulator circuit 122 modulates the receive signal with the acknowledgement message, and the antenna 102 transmits the modulated receive signal; said another way, the modulator circuit effectively modulates the NFC source signal with the acknowledgement message from the controller circuit 114. In summary, in response to the antenna 102 receiving the NFC source signal from the smartphone 440, the circuitry onboard the sensor patch 1200 powers up and sends, effectively via modulation of the NFC source signal, an acknowledgement/ready message or handshake to the smartphone so that the smartphone “knows” that the sensor patch is ready for the next step.

Then, at a step 2410 of the flow diagram 1600, the sensor-patch software application causes the smartphone 440 to transmit configuration data and commands to the sensor patch 1200. For example, the configuration data may configure the sensor patch 1200 to activate a blue one of the RGB LEDs 124 in response to a sensed temperature being below a temperature threshold Th₁, to activate a green one of the RGB LEDs in response to the sensed temperature being between Th₁ and another temperature threshold Th₂ inclusive, and to activate a red one of the RGB LEDs in response to the sensed temperature being greater than Th₂. Or, if the sensor patch 1200 has a numerical display as the reporter 126, then the configuration data configures the sensor patch 1200 to display the sensed temperature in ° C. or ° F. The caregiver 1101 may be able to enter or to select such sensor-patch behavior (and also one or more of the temperature thresholds Th₁ and Th₂) via a menu that the software application causes the smartphone 440 to generate on the display 2102 while the application is running or during the setup procedure described above in conjunction with FIG. 15. In response to the sensor-patch behavior entered or selected by the caregiver 1101, the software application causes the smartphone 440 to generate corresponding configuration data for the sensor patch 1200. And the commands may include respective commands to sense temperature, to provide an indication of the sensed temperature locally (e.g., via the RGB LED 124 per the above-described sensor-patch behavior), and to transmit the sensed temperature to the smartphone 440 for, e.g., display via the display screen 2102. To transmit the configuration data and commands to the sensor patch 1200, the smartphone generates one or more data and command signals and modulates the carrier of the NFC source signal with the data and command signals.

Next, at a step 2412, the sensor patch 1200 configures itself with the configuration data that the smartphone transmits per step 2410. The demodulator circuit 120 recovers the configuration data from the receive NFC receive signal from the antenna 102 by demodulating the NFC receive signal, digitizes the recovered configuration data, and provides the digital configuration data to the controller circuit 114 directly or via the memory circuit 108. In response to the configuration data and in a conventional manner, the controller circuit 114 configures itself and any of the other circuits that are configurable and that are to be configured in response to the configuration data. For example, the controller circuit 114, in response to the configuration data, sets the level of the regulated supply voltage that one or both of the power-supply circuit 118 and power-supply circuit 128 generate, sets the voltage level of the power signal that the energy-harvester circuit 116 generates, and sets the intensities of the RGB LEDs 124, the clock rate of the controller circuit 114 and other circuits onboard the sensor patch 1200, and the precision of one or both of the temperature sensors 1202 and 1204, and may disable one or more circuits and components of the sensor patch 1200 such as the ambient temperature sensor 1204. The controller circuit 114 can perform such configuration by loading the configuration data into one or more configuration registers of the controller circuit, of the memory circuit 108, or of any other circuit of the sensor patch 1200. And, if needed, the controller circuit 114 “reboots” or “restarts” itself, and possibly one or more other circuits of the sensor patch 1200, after loading of the configuration data.

Then, at a step 2414, the sensor patch 1200 executes any commands that the smartphone 440 transmitted and that the demodulator circuit 120 recovered. For example, a recovered command instructs the controller circuit 114 to execute a temperature-sensing routine. Further in example, such a routine includes the controller circuit 114 causing the object-temperature sensor 1902 to generate an object-temperature-sense signal, determining the sensed object temperature in response to the object-temperature-sense signal, and activating the RGB LEDs 124 in a pattern corresponding to the determined object temperature. In another example, such a routine includes the controller circuit 114 causing the object-temperature sensor 1202 to generate an object-temperature-sense signal and determining the sensed object temperature without activating the RGB LEDs 124 or the display 128 until hearing back from the smartphone 440 (see steps 2416-2418 below). In yet another example, such a temperature-sensing routine may include the controller circuit 114 causing both of the object- and ambient-temperature sensors 1202 and 1204 to generate respective object- and ambient-temperature-sense signals and determining the sensed object and ambient temperatures in response to the object- and ambient-temperature-sense signals, respectively, without activating the RGB LEDs 124 or the display 128 until hearing back from the smartphone 440 (see steps 2416-2418 below).

Next, at a step 2416, the controller circuit 114 sends the determined value of the sensed object temperature to the smartphone 440 via the modulator circuit 122 and the antenna 102 as described above in conjunction with FIGS. 1 and 12; if the controller circuit also determined a value of the sensed ambient temperature, then the controller circuit also sends the determined value of the sensed ambient temperature to the smartphone via the modulator circuit.

Then, at a step 2418, the smartphone 440 processes the values of the sensed object temperature and sensed ambient temperature received from the sensor patch 1200. Further to an example in which the controller circuit 114 determined the sensed object temperature and activated the RGB LEDs 124 in a pattern corresponding to the determined object temperature, the smartphone 440 displays the determined object temperature on the display 1402. Further to an example in which the sensor patch 1200 determined the sensed object temperature but did not activate a local display, the smartphone 440 determines, in response to an algorithm executed by the smart phone, the temperature of the child 1102 in response to the sensed object temperature, and displays the child's temperature on the display 1402; for example, the smartphone 440 determines that the child's temperature equals the value of the sensed object temperature as determined by, and received from, the sensor patch 1200, or determines that the child's temperature equals the value of the determined object temperature multiplied by a scalar or temperature-dependent factor. And further to an example in which the controller circuit 114 determined and sent values of both the sensed ambient and sensed object temperatures, the smartphone 440 determines, in response to an algorithm executed by the smart phone, a temperature of the child 1102 in response to the determined ambient and determined object temperatures, and displays the determined temperature of the child on the display 1402. In the above examples, the algorithm executed by the smartphone 440 may be coded as part of the software application as described above in conjunction with FIG. 15. And in addition to determining and displaying the child's temperature on the display 1106, the smartphone 440 also may save the child's temperature and a time at which the child's temperature was determined (the smartphone may provide the time or may receive a timestamp from the sensor patch 1200), and may generate and save a plot of the child's temperature over time. Alternatively, the smartphone 440 uploads the determined object and determined ambient temperature values, or the determined child's temperature, to the “cloud” for performance of any one or more of the above-described determinations attributed to the smart phone. Furthermore, such uploading of this information to the “cloud” may facilitate remote diagnosis and treatment (e.g., telemedicine) by the child's pediatrician or other medical professional.

Next, at a step 2420, the smartphone 440 sends information and one or more commands to the sensor patch 1200. Further to an example in which the sensor 1200 did not activate a local display at the time that it determined one or both of the object and ambient temperatures, the smartphone 440 sends the temperature of the child 1102 (as determined by the smart phone) to the sensor patch 1200 along with a command for the sensor patch to activate one or both of the RGB LEDs 124 and the display 126 to generate an indication of the child's temperature. Further in example, in response to the command, the controller circuit 114 causes the RGB LEDs 124 to radiate blue light if the determined child's temperature is below a first temperature threshold, to radiate a green light if the determined child's temperature is between the first temperature threshold and a second temperature threshold inclusive, and to radiate a red light if the child's temperature is greater than the second temperature threshold. The controller circuit 114 also may cause the display 126 to render the child's temperature numerically instead of, or in addition to, activating the RGB LEDs per the preceding sentence.

Referring to FIG. 16, alternate embodiments of the method represented by the flow diagram 1600 are contemplated. For example, the controller circuit 114 (FIG. 12) of the sensor patch 1200 can be configured to perform some or all of the operations attributed to the smartphone 440. Furthermore, the smartphone 440 and the sensor patch 1200 may error-code communications between them so that errors in a message can be detected or detected and corrected.

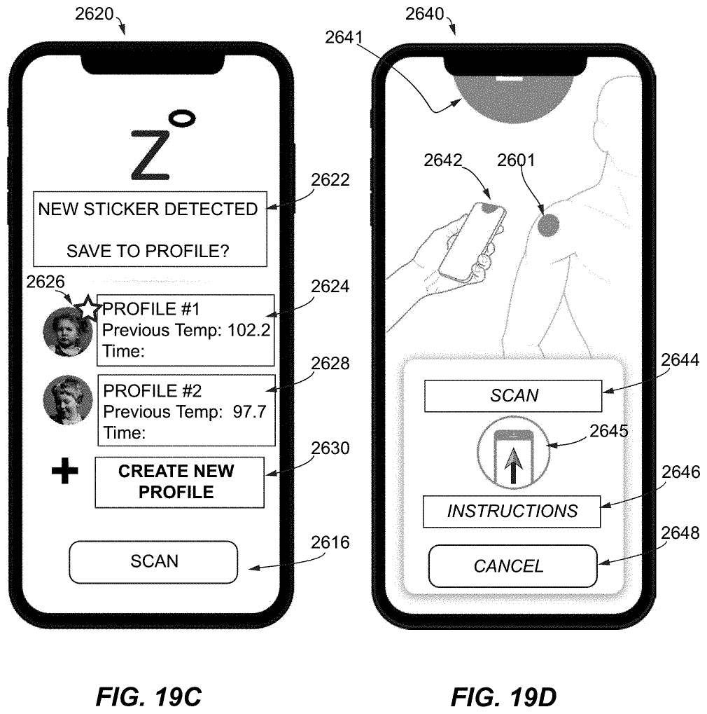

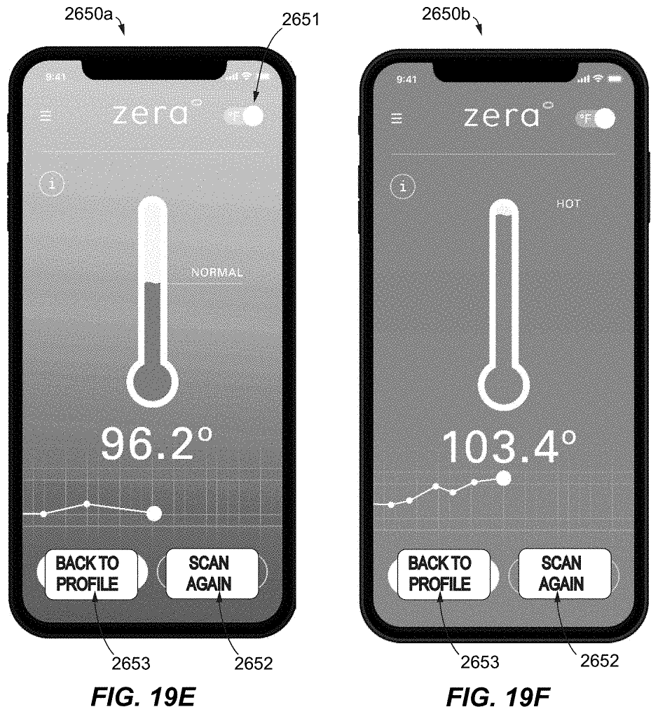

Moreover, while running the sensor software application, the smartphone 440 can use an onboard temperature sensor to determine the ambient temperature, in which embodiment the ambient temperature sensor 1204 may be omitted from the sensor patch 1200. In alternate embodiments (FIG. 17A), a patch sensor may include a duplex sensor having a thermal mass 1755 interposed between two thermistors S1 and S2, one in immediate thermal contact with the skin surface 1762 and another in thermal contact with a heat sink surface 1764 such as ambient air. The first temperature sensor S1 measures skin temperature, the second temperature sensor S2 measures the temperature at the heat sink interface. Each sensor is shown having three leads 1752,1754 as corresponds to the schematic of FIG. 2A. By measuring the two temperatures across a mass having known thermal conductivity, the temperature gradient ΔT in the thermal mass is more accurately indicative of the skin temperature when the thermistor temperature at S2 is known, and can be calculated by application of Fourier's Law for bodies at thermal equilibrium. As known, dQ/dθ=−kA(dt/dx) where dQ/dθ is the heat flux rate in any convenient units, A is a cross-sectional area of mass 1555, k is the thermal conductivity of the mass, and dt/dx is the temperature gradient across the mass (i.e., between sensor S1 and S2). Using the approach, a processor can be programmed to calculate skin temperature from the ΔT for a calibrated system where the thermal conductivity of mass 1755 is well characterized. A suitable algorithm to make the calculation of corrected temperature based on the differential temperature across a thermal mass of known resistivity is included in an application installed on the smart device or in a system server that receives the temperature data from the sensor patch.

Because the sensor patch is worn for extended periods and rapidly reaches thermal equilibrium, the equation can be resolved to a single variable (skin temperature) when ambient temperature is known and the heat transfer characteristics of the patch layers between a skin-contact thermistor and the outside air. While there may be convective differences that are not modelled completely, conductive heat loss characteristics permit calculation of a corrected body temperature at the skin side if ambient temperature is known and the heat transfer characteristics of the patch layers are well understood. A suitable algorithm to make the calculation of corrected temperature based on the differential temperature between the sensor patch and ambient temperature may be included in an application installed on the smartphone. A temperature sensor mounted in the smart device or in a radio hub provides the needed measurement of ambient temperature. In another embodiment, a second sensor patch may be placed away from the body, such as on a bedframe, and monitored by radio so that ambient temperature is entered when making a temperature correction. In a thermostated building, the room temperature may be known, and may be entered automatically or may be manually entered.

In yet another embodiment (FIGS. 17B, 17C), a secondary temperature sensor device 1770 is inserted into a USB port of the smartphone and software is installed to read the ambient temperature from the insertable sensor. The plug-in device 1770 is supplied as a kit with the patch sensor and is re-usable. The software will check the secondary temperature sensor reading periodically so that any body temperature readings from the patch sensor can be corrected for ambient temperature. When the plug-in device 1770 is used, the data and power ports of the USB port are converted into a circuit for measuring ambient temperature. No radio is required. The algorithm to make the calculation of corrected temperature based on the differential temperature between the skin temperature measured by the first sensor and the ambient temperature measured by the second temperature sensor are included in an application installed on the smartphone.

Users are advised not to perform temperature readings in direct sunlight or when the device is covered by clothing or other insulation, and a logic routine is included that detects and cautions against reliance on readings that are likely erroneous due to extreme external conditions.

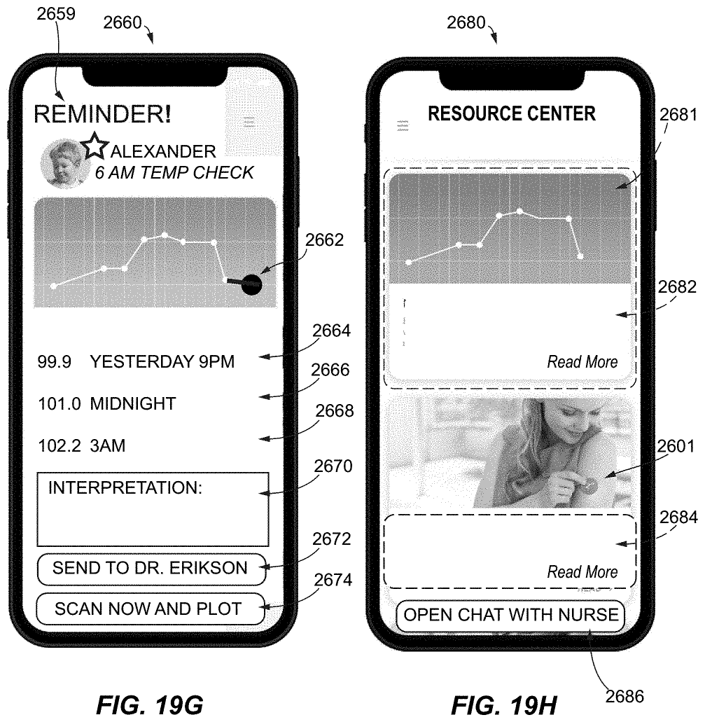

FIG. 18 is a diagram of smartphone circuitry 2500 of the smartphone 440 of FIG. 14, according to an embodiment. The smartphone circuitry 2500 includes a Bluetooth antenna 2502, a Bluetooth communications circuit 2504, a Wi-Fi antenna 2506, a Wi-Fi communications circuit 2508, an NFC antenna 2510, an NFC communications circuit 2512, a cellular antenna 2514, a cellular communications circuit 2516, a memory circuit 2518, one or more input devices 2520, one or more output devices 2522, and a controller circuit 2524.

The Bluetooth antenna 2502 can be a conventional antenna that is configured to transmit and to receive information or other signals that are compatible with the Bluetooth protocol.

The Bluetooth communications circuit 2504 can be a conventional communications circuit that is configured to convert information (e.g., data, commands, acknowledgements, and status in the form of a message or packet) from the controller circuit 2524 into one or more information or other signals that are compatible with the Bluetooth protocol and to provide these signals to the Bluetooth antenna 2502 for transmission; the Bluetooth communications circuit also is configured to recover information (e.g., data, commands, acknowledgements, and status in the form of a message or packet) from information and other signals received from the Bluetooth antenna and to provide the recovered information to the controller circuit. The Bluetooth communications circuit 2504 may include modulation circuitry, demodulation circuitry, error-coding circuitry, error-decoding circuitry, encrypting circuitry, and decrypting circuitry respectively configured to modulate a carrier signal with an information or other signal, to demodulate a carrier signal that is modulated with an information or other signal, to error code an information or other signal to be provided to the Bluetooth antenna 2502 for transmission, to error decode an information or other signal received from the Bluetooth antenna, to encrypt an information or other signal to be provided to the Bluetooth antenna for transmission, and to decrypt an information or other signal received from the Bluetooth antenna.

The Wi-Fi antenna 2506 can be a conventional antenna that is configured to transmit and to receive information or other signals that are compatible with the Wi-Fi protocol.

The W-Fi communications circuit 2508 can be a conventional communications circuit that is configured to convert information (e.g., data, commands, acknowledgements, and status in the form of a message or packet) from the controller circuit 2524 into one or more information or other signals that are compatible with the Wi-Fi protocol and to provide these signals to the Wi-Fi antenna 2506 for transmission; the Wi-Fi communications circuit also is configured to recover information (e.g., data, commands, acknowledgements, and status in the form of a message or packet) from information or other signals received from the Wi-Fi antenna and to provide the recovered information to the controller circuit. The Wi-Fi communications circuit 2504 may include modulation circuitry, demodulation circuitry, error-coding circuitry, error-decoding circuitry, encrypting circuitry, and decrypting circuitry respectively configured to modulate a carrier signal with an information or other signal, to demodulate a carrier signal that is modulated with an information or other signal, to error code an information or other signal to be provided to the Wi-Fi antenna 2506 for transmission, to error decode an information or other signal received from the Wi-Fi antenna, to encrypt an information or other signal to be provided to the Wi-Fi antenna for transmission, and to decrypt an information or other signal received from the Wi-Fi antenna.

The NFC antenna 2510 can be a conventional antenna that is configured to transmit and to receive information or other signals that are compatible with the NFC protocol; for example, the NFC antenna 2510 may be similar to the NFC antenna 102 of FIG. 13.

The NFC communications circuit 2512 can be a conventional communications circuit that is configured to convert information (e.g., data, commands, acknowledgements, and status in the form of a message or packet) from the controller circuit 2524 into one or more information or other signals that are compatible with the NFC protocol and to provide these signals to the NFC antenna 2510 for transmission; for example, the NFC communications circuit may be similar to the NFC communications circuit described above in conjunction with the smartphone 440 and in conjunction with FIGS. 1 and 12, and is configured to provide power to, and to communicate with, a sensor patch such as the sensor patch 1200 of FIG. 12 and any of the other sensor patches described herein. The NFC communications circuit 2512 also is configured to recover information (e.g., data, commands, acknowledgements, and status in the form of a message or packet) from information or other signals received from the NFC antenna 2510 and to provide the recovered information to the controller circuit 2524. The NFC communications circuit 2512 may include modulation circuitry, demodulation circuitry, error-coding circuitry, error-decoding circuitry, encrypting circuitry, and decrypting circuitry respectively configured to modulate a carrier signal with an information or other signal, to demodulate a carrier signal that is modulated with an information or other signal, to error code an information or other signal to be provided to the NFC antenna 2510 for transmission, to error decode an information or other signal received from the NFC antenna, to encrypt an information or other signal to be provided to the NFC antenna for transmission, and to decrypt an information or other signal received from the NFC antenna.

The cellular antenna 2514 can be a conventional antenna that is configured to transmit and to receive information or other signals that are compatible with a cellular protocol.

The cellular communications circuit 2516 can be a conventional communications circuit that is configured to convert information (e.g., data, commands, acknowledgements, and status in the form of a message or packet) from the controller circuit 2524 into one or more information or other signals that are compatible with a cellular protocol and to provide these signals to the cellular antenna 2514 for transmission; the cellular communications circuit also is configured to recover information (e.g., data, commands, acknowledgements, and status in the form of a message or packet) from information or other signals received from the cellular antenna and to provide the recovered information to the controller circuit. The cellular communications circuit 2516 may include modulation circuitry, demodulation circuitry, error-coding circuitry, error-decoding circuitry, encrypting circuitry, and decrypting circuitry respectively configured to modulate a carrier signal with an information or other signal, to demodulate a carrier signal that is modulated with an information or other signal, to error code an information or other signal to be provided to the cellular antenna 2514 for transmission, to error decode an information or other signal received from the cellular antenna, to encrypt an information or other signal to be provided to the cellular antenna for transmission, and to decrypt an information or other signal received from the cellular antenna.

The memory circuit 2518 can include one or both of conventional non-volatile and volatile memory, and is configured to store information such as data, configuration data, and the code of an operating system and of one or more software applications that the controller circuit 2524 is configured to execute. Furthermore, the memory circuit 2518 can be configured as a message buffer between the controller circuit 2524 and one or more of the communications circuits 2504, 2508, 2512, and 2516.

The one or more input devices 2520 are configured to allow a user to input data to the controller circuit 2524. Examples of the one or more input devices 2520 include a keypad, microphone, touch screen, and a universal-serial-bus (USB) port.

The one or more output devices 2522 are configured to allow the controller circuit 2524 to provide a user with information. Examples of the one or more output devices 2522 include a display screen, speaker, haptic device, and a USB port.

The controller circuit 2524 can include a conventional controller circuit such as a microprocessor or microcontroller, and is configured to communicate with the memory circuit 2518, one or more input devices 2520, one or more output devices 2522, and communication circuits 2504, 2508, 2512, and 2516 either directly or via the memory circuit. And the controller circuit 2524 can be configured to control one or both of the configuration and operation of one or more of the memory circuit 2518, one or more input devices 2520, one or more output devices 2522, and communications circuits 2504, 2508, 2512, and 2516.

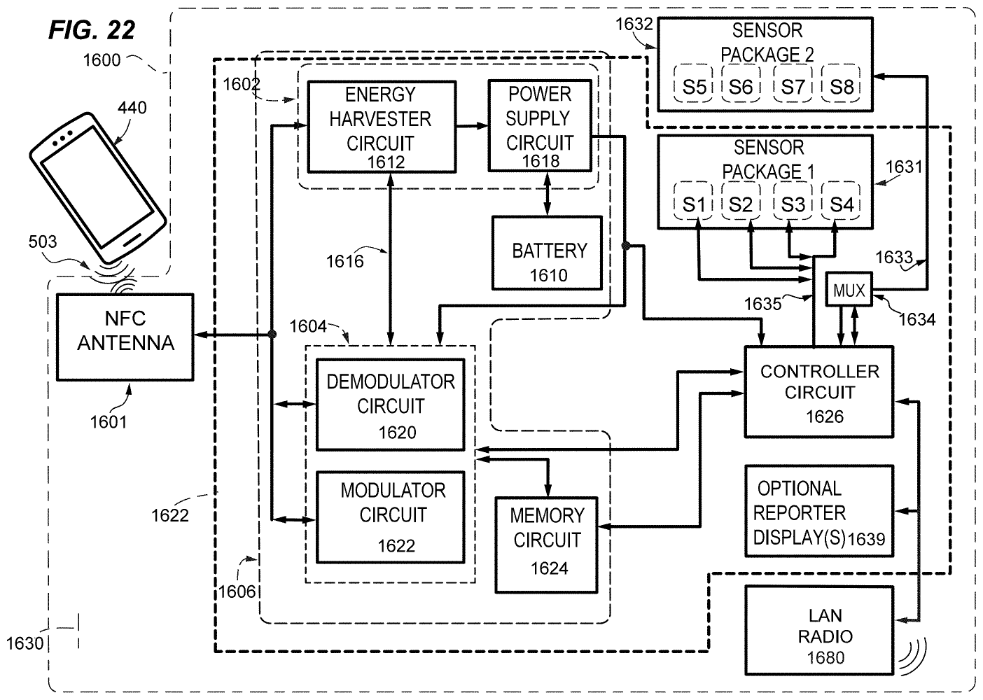

In operation, the controller circuit 2524 performs, our causes to be performed, the functions and operations herein attributed to a smart phone, such as the smartphone 2100 of FIG. 21. Furthermore, the controller circuit 2524 performs other functions and operations performed by conventional smart phones.

Referring to FIG. 18, alternate embodiments of the smartphone 440 and the smartphone circuitry 2500 are contemplated. For example, the smartphone circuitry 2500 may include an RFID antenna and an RFID communication circuit in place of, or in addition to, the NFC antenna 2510 and the NFC communication circuit 2512.

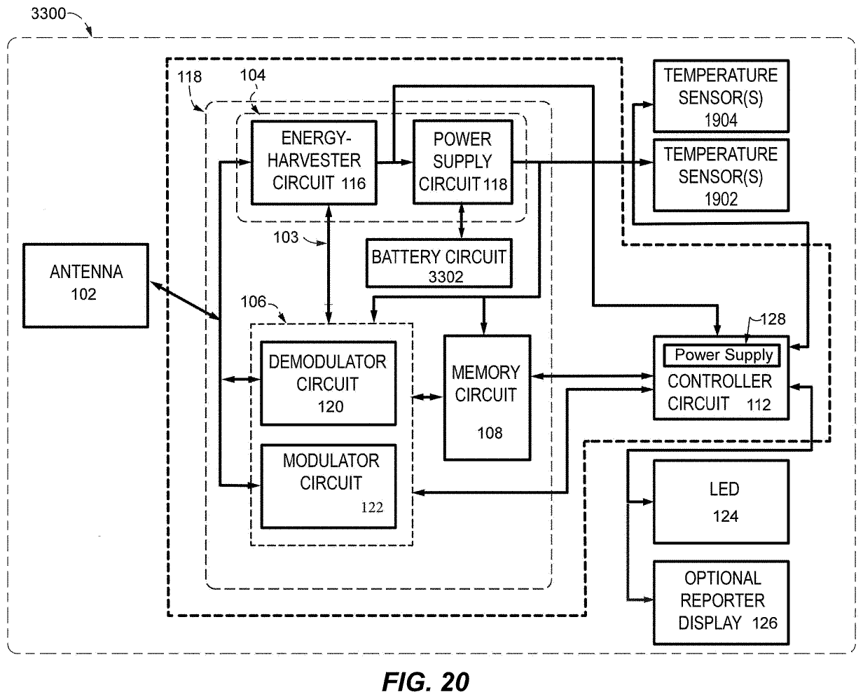

FIGS. 19A through 19H are screenshots from a display on a portable smart device. FIG. 19A is a view of a representative screenshot 2600. In this view, a menu of program options are shown. The view also shows an email address 2602 corresponding to the name and account of the user. Each user runs a personal copy of the program on their smart device; here a smartphone is shown. The smart device is used to power and take temperature readings from the sensor patches.

The program, also sometimes termed an “application”, is installed on the device in a non-transient computer memory. The application includes an instruction set, which when executed by a processor of the smart device, causes the display to show a graphical user interface (GUI). The GUI includes icons and information text and graphics useful in scanning the sensor patches to get temperature readings. The application also communicates the data to a central server, and may analyze the data collected using local computing resources or may display data analysis and interpretations received from the central server. The central server may use network resources and databases to issue notifications, alerts, reminders, and to log or archive data and will generally keep track of user account activity and any user-associated profile.

FIG. 19A shows several representative program functionalities. These include CHANGE ACCOUNT 2603, SCAN 2604, PROFILES 2605, FAQ 2606, USER MANUALS 2607, CHAT 2608, and REORDER 2609. The CHANGE ACCOUNT menu is useful if one user has several accounts or multiple users are set up to access the same NFC thermometers. Each account is named according to its email address 2602, according to one example.

The SCAN function is a command. Pressing the SCAN button 2604 causes the machine to cycle through a measurement cycle. A near field radio link is established with the sensor patch in proximity to the device, energy is exchanged, and the smart device extracts a temperature reading from the patch. But to perform a measurement, the patch is generally first associated with an individual profile, where the profile is constructed for each person for which temperature data will be collected. So for example, a daughter or son would first have a profile entered and the smart device would then couple with the nearest sensor patch. It would remember that thermometer so that in the future, readings from that thermometer are associated with the corresponding profile.

The PROFILE menu is used to build an identity for each person who will receive a wearable sensor patch. A snapshot of the person 2610 can be included as a quick graphical icon for faster access to the profile, for example. The profile will build a chronology of the temperature measurements and can contain notes such as physician contact information. The profile and data may be shared with other caregivers and family members. User's press the profile button 2605 to access personalized analytics capabilities of the system for each person wearing a sensor patch.

FAQ is a system resource, and the button 2606 actuates a browser. The browser displays answers to frequently asked questions that new users might have. Similarly, USER MANUALS is also a system resource, and accesses more detailed instructions and hardware specifications, including troubleshooting guides, for example.

Some users may need help, so a CHAT button 2608 may be provided. This provides the user with a shortcut to a cloud assistant, either an automated one or a human operator, who has familiarity with the device and can help with “How to?” questions or direct the user to other resources and also assist with account questions.

The user can use the REORDER button 2609 to order more patch sensors or accessories and to store credit card and shipping information. Generally, these services are provided by the cloud server to achieve an acceptable level of security. Biometric identity services or passwords may be required to access secure fields in the cloud server databases, according to one embodiment.

As indicated in FIG. 19B, profiles may also be accessed via a list of individual persons who have been assigned a sensor patch. Here each person is shown with a snapshot as an icon and the button next to the snapshot takes the user to a detailed view of the person's history and any personal information stored by the system. Profile 2611 may correspond to a daughter of a family unit, for example, and other profiles 2612, 2613, 2614, 2615 may correspond to other family members. Institutions may also assign profiles, and the profiles may be linked to custom software for direct charting into a central hospital database in another embodiment. A button 2615 takes the user to a GUI page designed for entering new profile data. Once the system profile is set up and an active profile is selected (as indicated by the star next to an icon), pressing the SCAN button 2616 will cause a temperature reading for the sensor patch in NFC proximity, and the temperature data to be associated with profile 2611, for example.

The profile screen can also be actuated with a new sticker is detected, as shown in FIG. 19C. In screenshot 2620, an alert 2622 pops up, advising the user that a new sensor patch has been detected, and asking the user to assign the patch to an existing profile selected from, for example, profiles 2624 or 2628, or create a new profile using button 2630. The star at 2626 indicates which active profile will receive the temperature data. Once the assignment is complete, a SCAN can be started by pressing button 2616 as shown in the earlier figures.

FIG. 19D is a view of the “START SCAN” screenshot 2640. As currently practiced, the sensor patch 2600 is applied to the shoulder of the subject of the measurement. The person for whom the temperature data will be scanned has a profile, and this screen now prepares the user to start the scan. Pressing button 2616 will take the user to screen 2640. Pressing button 2644 will start a scan and record a result.

In FIG. 19D, a tutorial is demonstrated. The user is shown how to hold the smart device (2642, which supplies near field energy to the sensor patch 2601). The upper edge of the device 2641 is held near the patch 2601 so that the patch remains visible and the excitation and receiving coils can electromagnetically couple. While smart devices may have alternative NFC coil architecture, commonly available smart devices such as the Samsung Galaxy smartphones position the large NFC transceiver in about the center of the back panel of the device. So that the position shown provides unobstructed transmission of radio energy between the coils in the smart device and the coil in the patch sensor, the smart device may be held by the bottom edges if desired, but it is useful to be able to monitor both the screen of the smart device and to observe the patch sensor during a measurement.

Display element 2645 can be an instruction to hold the device closer. The arrow can pulse. Or in another embodiment, the arrow can pulse more quickly if the energy coupling is insufficient, and can become green and steady when coupling is sufficient. Similarly, the display area 2641 can provide feedback to the user if the coupling is sufficient or not sufficient by a change in color. More information or help can be accessed by pressing the INSTRUCTIONS button 2646. If the user has to abort the measurement because measuring conditions are inadequate or the sensor patch has to be replaced, for example, the user can cancel the procedure by pressing CANCEL button 2648. Generally, once a user has successfully completed a couple measurements, the procedure is simple enough, and the user can simply bring the smart device close to the sensor patch in one hand and press the SCAN button 2644 in the other hand. In a few seconds, once the measurement is completed, a temperature result screen will be displayed.

FIGS. 19E and 19F show temperature result screens 2650a, 2650b, respectively. In a first view, FIG. 19E shows a “normal” temperature reading. Ideal normal body temperature is 98.6° F. (37° C.). The display 2650 reports a temperature of 96.2° F. (Fahrenheit mode 2651, is selected in the upper right corner). A plot of recent temperature measurements in the profile may also be displayed if data is available. The user has the option to repeat the measurement (2652, SCAN AGAIN) or to go BACK TO PROFILE (button 2653) in order to complete the reporting and analysis of the result. The color of the screen can correspond to an overall clinical interpretation of the result, green for example can indicate a temperature lacking in suspicion of fever, and orange or red can indicate a fever or condition raising concern. Thus, for comparison, screenshot 2650a can be green, and screenshot 2650b can be red, the red indicating a fever. In FIG. 19F, a temperature of 103.4° F. is demonstrated. When a measurement is made, the time and location is also recorded in the corresponding profile.

FIG. 19G shows a REMINDER screenshot 2660. In this instance, following doctor's orders, the caregiver has been instructed to measure a boy's temperature at 3 hr intervals. The smart device enables the user to set an alarm so that a reminder notification will be displayed at about 6 AM. The pot shows temperature results on the previous day at 9 PM, 12 PM, and 3 AM in white. When the 6 AM measurement is completed, it will be plotted as the next datapoint 2662. Data is also tabulated for reference. Here the system causes the smart device (or data may be stored in local memory) to display the 9 PM temperature 2664, the midnight temperature 2666, the 3 AM temperature 2668, and also provides room for an INTERPRETATION panel 2670 or notes. Functions include a button 2672 that will cause the data and plot to be sent to the family's doctor and another button 2674 to that will start the 6 AM scan when the smart device is in position next to the sensor patch and the user is ready.

FIG. 19H is a view of a RESOURCE CENTER screenshot 2680. The screen includes a sample plot 2681 and guides to interpretation 2682, including references for more reading. More information 2684 may also be provided about the sensor patch 2601, as demonstrated here affixed on the shoulder of a model.

The sensor patch 2601 may include an LED that will illuminate during or at the completion of the measurement. The color of the LED may be indicative of the temperature, as when an RGB-LED is used. The LED illumination is transitory, because it depends on power received from the smart device. In more advanced embodiments, the patch may have memory, such as Z-RAM, that enables the device to store a limited number of subsequent measurements in its memory. That enables the user to access a series of measurement without the need to identify the correct profile, or serves as a check that the profile selected corresponds to the sensor patch selected.

In another option, screenshot 2680 shows a button 2686 that is a shortcut to starting a conversation, either as a virtual chat or as a live consultation, with a nurse practitioner or other healthcare practitioner, if advice is needed about how to manage the person's condition. This might be displayed if the temperature result is abnormal, if there are predisposing conditions such as asthma that complicate clinical management, and if there is staff available to help relieve user's concerns.

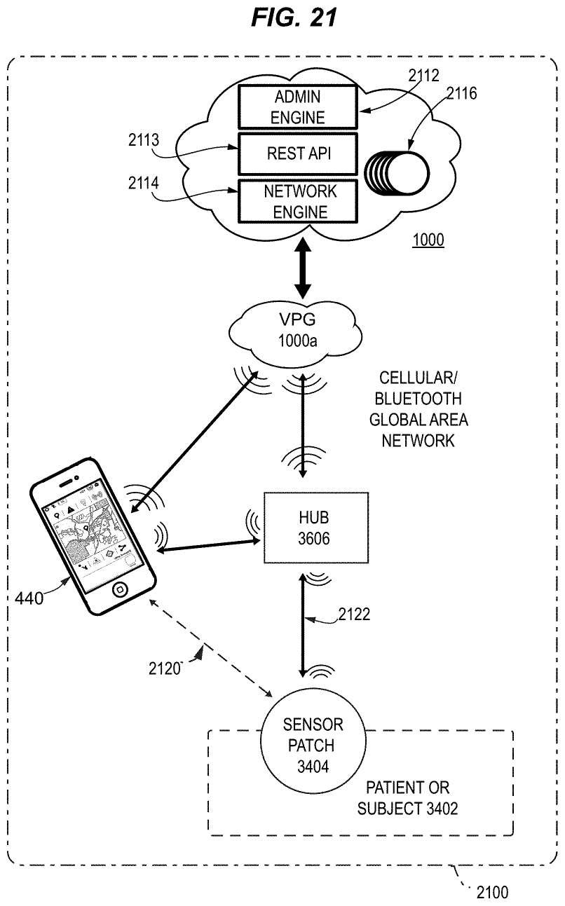

FIG. 20 is a circuit diagram of a sensor patch or bandage 3300 (hereinafter “sensor patch 3300”), according to an embodiment in which the sensor patch 3300 is similar in circuit topology, structure, and operation to the sensor patch 1200 of FIG. 12, and where like numbers reference components common to FIGS. 1, 12 and 20. Differences between the sensor patch 3300 and the sensor patch 1200 include that the sensor patch 3300 includes a battery circuit 3302.

The battery circuit 3302 includes a battery and a charging circuit configured to charge and recharge the battery. For example, the battery can be a thin-film printed manganese or other type of battery. And the charging circuit can be configured to generate a charging signal in response to the regulated supply voltage that the power-supply circuit 118 generates while the energy-harvester circuit 116 is extracting power from an NFC source signal, and to charge and recharge the battery with the charging signal. For example, the charging circuit can be configured to limit the charging current to a suitable value (include zero) that is dependent on the voltage across the battery while being charged or recharged. And in response to the absence of an NFC source signal, the battery circuit 3302 is configured to disable the charging circuit and to couple the battery to the power supply circuit 118, which is configured to generate the regulated supply voltage from the battery voltage.

Referring to FIG. 20, alternate embodiments of the sensor patch 3300 are contemplated. For example, the battery circuit 3302 also can be configured to provide the battery voltage to the power supply 128 of the controller circuit 112 in response to the absence of an NFC source signal. Furthermore, the battery circuit 3302 can be omitted such that the battery is coupled to the power supply circuit 118, which can be configured to generate a regulated supply voltage in response to the battery voltage in the absence of the NFC source signal or in an embodiment in which the sensor patch 3300 is powered only by the battery and not by any wireless source signal. Moreover, features of embodiments described in conjunction with FIGS. 1-19 and 21-37 may be applicable to the sensor patch 3300 of FIG. 20.

FIG. 21 illustrates a system 2100 by which sensor data from a sensor patch 3404 is uplinked via an intermediary hub 3606 to a cloud host 1000. In this instance, a virtual private gateway 1000a is used to improve the security of the uplink. The transmissions may be bidirectional and may include commands and updates on the downlink. The hub 3604 can substitute for smartphone 440 in networking with a cloud host, but can also supplement the smartphone by increasing the range that data received from a sensor patch can be forwarded to a smartphone or to the cloud. By operating the sensor patch with a LAN radio with low energy transmission capability, the hub 3606 acts as a repeater and booster to extend the range of the signal. The hub may also be configured to encrypt the signal so that privacy of medical information, for example is protected.

The hub device 3406 is configured to automatically, or on command, power, activate, and receive sensor readings from the sensor patch 3404. Although a medical professional, such as a doctor or a nurse, can power, activate, and receive sensor readings from the sensor patch 3404 with a hand-held smart device such as a smartphone 440 via optional radio link 2120, this may be limiting when continuous monitoring is needed, and increases the number of times that the medical professional is in close proximity to the patient 3402 (physical proximity between a medical professional and a patient may not be desirable if, for example, the patient is infected with a communicable contagion such as COVID19). The device 3406 allows remote sensing and reporting of a measured quantity or condition (e.g., body temperature) of the patient 3402 on command or automatically. Typically the radio link 2122 can be a low energy Bluetooth channel or Manet signal that has a range of a few meters, such as would be sufficient for a broadcast that is limited to a radio proximity no larger than a bedroom. The range broadcast by the hub 3606 can be much larger, several hundred meters for example, and the hub can serve not only to relay data back and forth from the patch to the medical professional, but can also serve to locate the patient by providing coordinates or mapping function as displayed on smart device 440, or can be a radio tether that will alarm if the patient leaves its radio proximity. In normal use, the radio tether 2122 will be intact and the location of the patient is associated with the location of hub 3606.

While the sensor patch can have a very low power radio, or a passive NFC radio capacity, the hub may be AC powered and may include one or more LAN radios, including for example one or more of WiFi, UWB, BT and Cellular radios. Zigbee and Thread radio protocols may also be implemented. The hub will also broadcast a radio unit identifier that can be associated with a particular patient 3402, and can include security features for protecting medical information.

Sensor patches 3404 can include a selection of transducers for monitoring vital signs, such as those described in US Pat. Publ. No. 2005/0261598 to Banet, for example, which is incorporated in full by reference. Optical and electrical sensor inputs may be combined with temperature and pressure measurements to provide a more complete snapshot of patient condition and wirelessly transmit the information to a central nursing station, a physicians PDA, or a cloud host for analysis and archiving.

Consequently, the hub 3406 allows remote monitoring one or more conditions (e.g., temperature, other vital signs) of the patient 3402 without cumbersome leads that are connected between the device and the patient and that limit patient movement and comfort. This system 2100 constitutes essentially an IOT network for operation in a clinic or hospital.

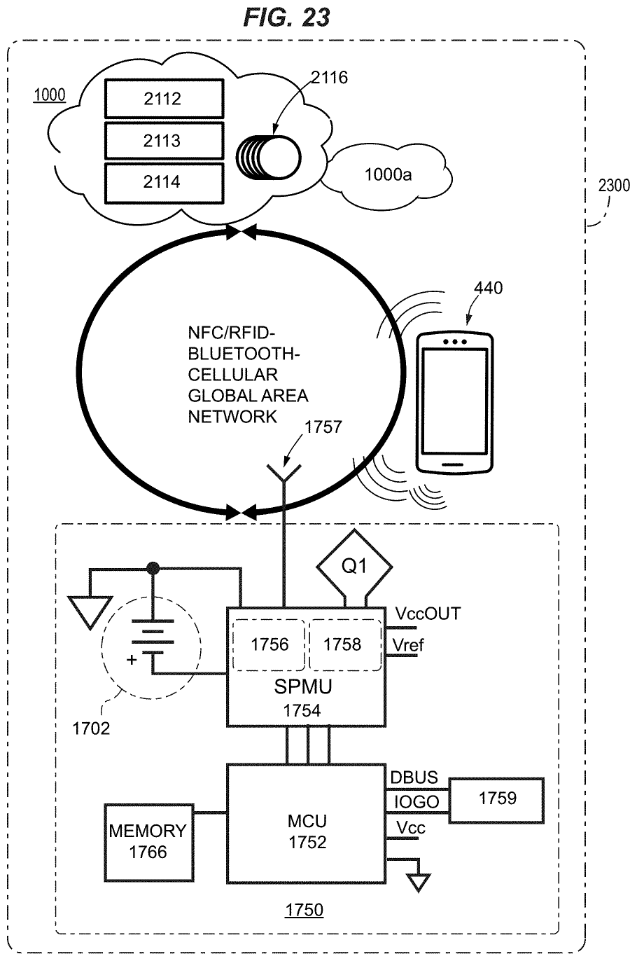

FIG. 22 is an exemplary sensor patch device 1600 with circuit functioning as an NFC/RFID and wireless digital temperature logger in cooperation with a smartphone 440 or hub 3406. The circuit includes a LAN radio 1680 (such as a BTLE radioset) powered and controlled via a controller circuit 1626 via power supplied from battery 1610.

The circuit may also include a switching regulator for selecting an alternate power source when battery power is lost. Power can be supplied via a V_{cc} rail for example, directly from the battery or as regulated by a microcontroller or power conditioning and management unit of a power supply circuit 1618, for example, where power from an NFC/RFID antenna 1601 may be harvested for passive operation without battery. In this way, the device is a hybrid dual power device that is operative in communicating a variety of sensor data to an IoT smart network either by modulation and demodulation of an NFC/RFID radio signal 503 or using the LAN radio 1680. Whereas NFC/RFID radio operation is limited to short range, Bluetooth or WiFi radio for example, operates at distances up to 100 meters or more, and is well suited for indoor use, such as in an ICU of a hospital, where multiple sensor patches are monitored from a remote nursing workstation. Passive power via NFC/RFID may be supplied from a smartphone 440, for example. The smartphone 440 or hub 3406 (FIG. 21) may also function to forward Bluetooth signals to a remote server. In place of the smartphone, a wireless hub may be used, and the hub may include an accessory sensor for measuring ambient temperature.

The circuit diagram of FIG. 22 shows sensor patch 1600 configured for deriving operating power from an NFC/RFID signal 503 generated by a proximate NFC/RFID smart device 440, for reporting one or more sensed quantities and conditions in a local display mode, and for exchanging information with a proximate NFC/RFID smart device by modulating and demodulating the NFC/RFID signal, according to an embodiment.

The antenna 1601 is configured to couple the received NFC/RFID signal to (a) a power circuit 1602, and (b) a communication circuit 1604, which is configured to perform bidirectional NFC/RFID communications between the smart device 440 and the sensor patch 1600. Power harvesting and data exchange in circuit block 1606 can occur simultaneously.

The power circuit 1602 includes an energy-harvester circuit 1612 and a power-supply circuit 1618. Together, the circuits 1612 and 1618 include a half- or full-wave rectifier, a voltage regulator, and a circuit (e.g., a POR circuit) configured to indicate when a supply voltage generated by the power-supply circuit 1618 equals or exceeds a threshold voltage (e.g., 1.8 V). The power-supply circuit 1618 is configured to power, with the supply voltage, at least the power circuit 1602, the communication circuit 1604, and a controller circuit 1626 (e.g., a microprocessor or microcontroller that can include one or more of a memory cache, configurable (e.g., with configuration data such as firmware) logic circuitry, and clock circuit).

Note that the energy-harvester circuit 1612 can include a bidirectional link 1616 with the communication circuit 1604, and the link may be useful for initial setup of a radio link during power up of the sensor patch 1600. Circuit 1606 includes both the power-harvesting and information-exchange circuits of the sensor patch 1600.

As stated above, the energy-harvester circuit 1612 and power-supply circuit 1618 supply power to the communication circuit 1604, which includes a demodulator circuit 1620 and a modulator circuit 1622 as already described. The demodulator circuit 1620 is configured to recover information, such as commands or data, that the smart device 440 transmits and the antenna 1601 receives and provides to the communications circuit 1604. And the modulator circuit 1622 is configured, effectively, to modulate the source NFC/RFID signal (i.e. acting as a variable “load”), via the antenna 1601, with information, such as status or measurement data, generated by the controller circuit 1626 for transmission to the smart device 440.

A memory circuit 1624 includes one or both of volatile and non-volatile memory. For example, the non-volatile memory can be configured to store configuration data for configuring one or more of the circuits of the sensor patch 1600, and a set of software instructions that, when executed by the controller circuit 1626, cause the controller circuit, or one or more circuits under the control of the controller circuit, to execute a sensor reading, reporting, and notifying cycle. The volatile memory of the memory circuit 1624 can include registers and buffers configured for storing data received from the smart device 440 via the demodulator circuit 1620 and data received by the controller circuit 626 for transmission to the smart device 440 via modulator circuit 1622.

Wireless-communication circuit 1606 may have an integrated-circuit architecture (i.e., may be an ASIC). The integrated circuit includes the power circuit 1602 and the communication circuit 1604. The communication circuit 1604 is configured to configure and to control the energy-harvester circuit 1612 by loading configuration data from the memory circuit 1624 into the power circuit 1602, and also may obtain information from the power circuit 1602 via link 1616 (hence the bidirectional arrow/coupling between the energy-harvester circuit 1612 and the communication circuit 1604). For example, the wireless-communication circuit 1606 is a single-chip component and is configured to operate with some level of independent functionality from the controller circuit 1626. The circuit 1606 is configured to generate an onboard voltage sufficient to wake up the controller circuit 1626, for example, before the controller circuit can take a sensor measurement.

The power circuit 1602 is configured to harvest power from the NFC/RFID receive signal generated by the antenna 1601, to provide the harvested power to other circuits and components of the sensor patch 1600, and includes an energy-harvester circuit 1612 and a power-supply circuit 1618. The energy-harvester circuit 1612 is configured to convert the receive signal from the antenna 1601 into a raw power signal. For example, the energy-harvester circuit 1612 includes a conventional half-wave or full-wave rectifier that is configured to generate a rectified signal, and one or more low-pass filters configured to generate the raw power signal by reducing the ripple superimposed on the rectified signal. And the power-supply circuit 1618 is configured to convert the raw power signal into a regulated power signal having a regulated voltage, for example, in an approximate range of 1.8 Volts (V) to 3.6 V. For example, the power-supply circuit 1618 can be any suitable type of voltage regulator, such as a linear regulator, a buck converter, a boost converter, a buck-boost converter, or a flyback converter with low drop out switching regulator, for example.

The communication circuit 1604 is configured to recover source information from the NFC/RFID source (e.g., smart phone, 440) that generates the NFC/RFID source signal, is configured to transfer sensor-patch information from the sensor patch 100 to the source, and includes a demodulator circuit 1620 and a modulator circuit 1622. The NFC/RFID source is configured to transmit source information, such as source commands or other source data, to the sensor patch 1600 by modulating a carrier signal with the source information to generate the source signal; that is, the source signal is configured to provide power, and to carry source information, to the sensor patch 1600. For example, the source is configured to generate the source signal by amplitude, frequency, or phase modulating a sinusoidal carrier signal with a source information signal that represents the source information; further in example, the source is configured to generate the source signal by amplitude-shift-key (ASK) modulating a sinusoidal carrier signal with a source information signal that represents the source information. The demodulator circuit 1620 is configured to recover the source information from the receive signal (received from the antenna 1601) by demodulating the receive signal to recover the source information signal, by further processing (e.g., error decoding) the recovered source information signal, and by digitizing the recovered and further processed source information signal. Alternatively, if further processing of the recovered source information signal is not needed (e.g., the source did not error code the source information signal), then the demodulator circuit 1620 can be configured to omit the further processing of the recovered source information signal. Furthermore, the modulator circuit 1622 is configured to send sensor-patch information, such as a sensor-measurement value (e.g., temperature), sensor-patch status, or other sensor-patch data, to the source by modulating the source signal with the sensor-patch information; that is, in addition to providing power and carrying source information from the source to the sensor patch 1600, the source signal also carries sensor-patch information from the sensor patch to the source. For example, the controller circuit 1626 is configured to generate a digital sensor-patch-information signal that represents the sensor-patch information, and the modulator circuit 1622 is configured to amplitude, frequency, or phase modulate the source signal with the sensor-patch information signal via the antenna 1601; said another way, the modulator circuit 1622 effectively modulates the carrier signal generated by the source with the sensor-patch information signal. For example, the modulator circuit 1622 is configured to amplitude-load-modulate (ALM) the carrier signal generated by the source with the sensor-patch information signal. The controller circuit 1626 or the modulator circuit 1622 also can be configured to further process the sensor-patch information signal by, for example, error-encoding the sensor-patch information signal before the modulator circuit modulates the source signal. The source typically includes a demodulator circuit, which may be similar to the demodulator circuit 1620, configured to recover the sensor-patch information by demodulating the source signal to recover the sensor-patch information signal, by further processing (e.g., error decoding) the recovered sensor-patch information signal, and by digitizing the recovered and further processed sensor-patch information signal. Alternatively, if further processing of the recovered sensor-patch information signal is not needed, then the source can be configured to omit the further processing of the recovered sensor-patch information signal. Therefore, the above-described configurations of the source and the sensor-patch 1600 allow the source and sensor patch to communication with one another bidirectionally over a carrier signal that the source generates. To prevent the source and sensor-patch information signals from interfering with one another, the source and the sensor patch 1600 can be configured to implement one or more conventional interference-prevention techniques. For example, the source and sensor patch 1600 can be configured to implement time-division multiplexing such that the sensor patch 1600 does not modulate the source signal with the sensor-patch information signal while the source is modulating the source signal with the source information signal, and the source does not modulate the source signal with the source information signal while the sensor patch is modulating the source signal with the sensor-patch information signal. In such an embodiment, the NFC/RFID source (e.g., a smart device such as a smart phone) may be the master and indicate, via the source information signal, when the sensor patch 1600 can, and cannot, modulate the source signal with the sensor-patch information signal. Or, if the source and the sensor patch 1600 are configured to implement frequency modulation, then the source and the sensor patch each can be configured to frequency modulate the carrier signal (which is generated by the source) at significantly different respective modulation frequencies. In addition, the communication circuit 1604 is configured to receive, and to be powered by, the regulated supply voltage generated by the power-supply circuit 1618.

In an embodiment, the controller circuit 1626 is configured to perform at least two functions. Sensor banks 1631 and 1632, which may be mounted on a flexible circuit support membrane, e.g., a substrate, 1630 with the other circuits and components of the sensor patch 1600, are in digital communication with the controller circuit 1626. The sensors may be analog or digital, but the sensor package(s) for sensors S5, S6, S7 and S8, if these sensors are analog, can include an ADC to render these sensors compatible with a bus, such as an I²C bus, 1635.

As shown, it is contemplated that sensor patch 1600 may include multiple sensor packages (1631,1632) and that the sensors and LAN radio may receive a regulated supply voltage either from a battery 1610 or from an energy harvester circuit 1612.

The sensor patch 1600 is configured for passive or active operation as a data logger from sensor packages 1631 and 1632, each of which may contain multiple sensors S1 through S8, for example.

To access sensor readouts for sensors S1, S2, S3, and S4, a serial bus 1633 joined to a first bank 1631 of these sensors may include a multiplexer 1634 or multiple sensors joined to an I²C bus may include an I²C multiplexer switch on the bus. The controller circuit 1626 is configured to receive sensor output and to process the sensor output for several purposes.

In a first executable step, the controller circuit 1626 may process the sensor output according to programmable rules established by the manufacturer or programmable by the user, and transmit a command to a reporter component 1639.

In a second executable step, the controller circuit 1626 may include several data fields in a message and cause the data string to be transmitted via the modulator circuit 1622 through antenna 1601 to a proximate compatible smart device 440. The message includes a digital unique identifier UID that is assigned to the sensor patch 1600. A time stamp may also be included. From the smart device 440, the “notification” may be forwarded to LAN or WAN network components and to any remote workstation by a Bluetooth signal, by a cellular signal, by Wi-Fi, or by any wireless or wired system. The smart device 440, or a remote workstation in receipt of data may also present display tables and trendlines that show the latest sensor measurement in the context of past sensor measurements. By storing the data in a folder dedicated to the unique identifier UID of the sensor patch 1600 and associating that folder with a particular target of the measurements (e.g., a patient, a sick child, a champagne bottle, a refrigerator, a package, a truck trailer, a runner in a race, a soldier, a blood specimen, and so forth, without limitation) so that a permanent tracking history of the measurement is incorporated into the folder history, then that folder can be copied to other parties having an interest according to permissions and rules associated with the UID by the user or by a system administrator.

Each of the one or more sensors is configured to sense a respective physical quantity or condition such as temperature of an object (e.g., a human forehead) to which the sensor patch 1600 is attached, a level of ambient light to which the sensor patch is exposed, a level of humidity to which the sensor patch is exposed, or a linear movement (e.g., acceleration), angular movement (e.g., angular velocity), or a vibration (e.g., sound) that the sensor patch experiences, and to generate a respective analog or digital sense signal that represents a value of the sensed quantity or condition. For example, a sensor S1 can include a conventional thermistor circuit configured to sense a temperature and to generate a voltage or current having a magnitude, phase, or frequency that represents, or that is otherwise related to (e.g., proportional to, inversely proportional to), the value of the sensed temperature. The one or more sensors can be configured to receive, and can be configured to be powered by, the regulated power signal that the power-supply circuit 1618 is configured to generate, or can be configured to be powered by a signal generated by the controller circuit 1626 (e.g., by a reference power-supply circuit integral to and onboard the controller circuit).

The reporter display(s) 1639, or indicator circuit (also “indicator”) is configured to indicate locally, in response to the one or more sense signals generated by the one or sensors, a value of a physical condition or quantity that the one or more sensors sense, or a range in which the value is located. The reporter circuit 1639 may include one or more light-emitting diodes (LEDs), such as a red-green-blue (RGB) LEDs display, and can also include another reporter circuit, such as an alphanumeric display (e.g., a liquid-crystal display (LCD)), a sound generator, chromogenic ink, or a vibration (haptic) generator. An RGB LED display is configured to generate a light having a color indicative of a range in which the value of the sensed physical condition or quantity is located. For example, if one of the sensors S1 is a temperature sensor configured to sense a temperature of a human body and considering that 98.6° Fahrenheit (° F.) is considered to be a typical core body temperature of a healthy human, an RGB LED display can be configured to generate blue light if the temperature that the sensor senses is below 97.6° F., to generate a green light if the temperature that the sensor senses is within the range 97.6° F.-99.6° F. inclusive, and to generate a red light if the temperature that the sensor senses is greater than 99.6° F. Further to this example, another reporter can be an alphanumeric display configured to display the sensed temperature, for example, “97° F.” Or, another kind of indicator can be a piezoelectric crystal configured to generate a sequence of sounds or vibrations that is indicative of the sensed temperature. For example, the piezoelectric crystal can be configured to generate a single sound or vibration if the temperature that the sensor senses is below 97.6° F., to generate two sounds or vibrations if the temperature that the sensor senses is within the range 97.6° F.-99.6° F. inclusive, and to generate three sounds or vibrations if the temperature that the sensor senses is greater than 99.6° F. Or, the piezoelectric crystal can be configured to “play” a first tune if the temperature that the sensor 1510 senses is below 97.6° F., to play a second tune if the temperature that the sensor senses is within the range 97.6° F.-99.6° F. inclusive, and to play a third tune if the temperature that the sensor senses is greater than 99.6° F. And it is understood that an LED display and any other reporter being “configured to” perform a respective function includes the controller circuit 1626 being configured to cause the LED display and the other reporter to perform the respective function. The reporter circuit 1639 can be configured to receive, and to be powered by, the regulated power signal that the power-supply circuit 1618 is configured to generate, or can be configured to be powered by a regulated voltage signal generated by the controller circuit 1626.

Moreover, although one of the one or more sensors S1 is described as being a temperature sensor, the one or more of the sensors of sensor packages 1631 and 1632 can be any suitable type of sensor, such as acoustic (e.g., piezoelectric, microphone), optical (e.g., photocell for reflected light, CMOS pixel array), and chemical (e.g., to detect substances in sweat) sensors, multi-axis accelerometers, multi-axis gyroscopes, and microelectromechanical (MEMs) devices such as MEMs cantilevers. For example, a displacement sensor can be configured to sense a heartbeat in a peripheral artery of a human by detecting a transient increase in displacement. The controller circuit 1626 can readily analyze characteristics of the displacement to calculate heart rate, but also can analyze other characteristics to determine cardiac output such as left-ventricular ejection volume. And in extreme cases of congestive heart failure, the sensor patch 1600 can be configured to report a local alarm state and to notify a remote dispatcher, a nursing station, or a bedside monitor that assistance is required. In addition, although described as being a microcontroller or microprocessor, the controller circuit 1626 can be, or can otherwise include, any suitable circuit, such as a digital signal processor (DSP), an application specific integrated circuit (ASIC), a field-programmable gate array (FPGA) or other programmable logic device, discrete gate or transistor logic, discrete hardware components, or any combination thereof designed to perform the functions described herein. Furthermore, although described as extracting power from, and communicating via, an NFC/RFID signal, the sensor patch 1600 can be configured to extract power from, and to communicate via, a radio-frequency-identification (RFID) signal. Moreover, a reporter LED may be omitted, the reporter display 1639 may be a chromogenic ink or a similar device that has a color corresponding to a temperature of an object to which the sensor patch 1600 is in contact, and the sensor patch is configured to send a value of a sensed or determined temperature to the smart device generating a wireless source signal for numerical display of the value; therefore, the sensor patch 1600 is configured to indicate a sensed or determined temperature with little or no power draw by the reporter display(s) 1639.

The controller circuit 1626 also may command any optional reporter component (such as a buzzer or vibrator) to call attention to the data. The reporter circuit 1638, which can include one or more RGB LEDs, or the optional one or more reporter displays 1639, can serve as an alarm if there is a critical sensor result, or can signal an “all clear” if the data is within expected limits.

In variants of this architecture, the sensor patch 1600 is fitted with an array of sensors, such as force sensors. The result is that by addressing each sensor by a particular address, such as on an I²C bus, and by time stamping each sensor output, a temporal and spatial map of deformations in the sensor patch 1600 can be constructed. Such a force-sensing operation may have interest in clinical applications where heart pulse rate and pulse characteristics are studied at a peripheral artery. The sensor array also permits a user to place the sensor patch 1600 on top of an artery, such as on the wrist, without exactly knowing where the radial artery is—the sensor patch 1600 can be configured to detect the strongest signal and assess pulse rate accordingly. Similarly, the sensor patch 1600 may be configured to assess peroneal, brachial, or carotid pulse without detailed palpation to determine the precise anatomy of the strongest signal. And by comparing pulse deformation along a series of sensors that follow the artery, the sensor patch 1600 can integrate the size of the pulse wave. The integration, when placed in the context of a database of measurements made of patients in various stages of heart pathology or in treatment for heart pathology, can be used to make predictions about diagnosis, about prognosis, about the response to therapy, and can be used to alert the user (via the patch reporter) or a caregiver or administrator (via a patch notification) that something is amiss, that some new event is occurring, or that a series of measurements over time shows a steady improvement or a worsening condition. When confined with electrophysiology of the heart by use of an electrocardiogram (EKG), measurement of peripheral pulse volume can provide a convincing indication of cardiac ejection volume, a major predictor for morbidity and mortality in congestive heart failure. Thus, use of the sensor patch 1600 in combination with a smart device for a daily home examination, when transmitted to an experienced clinician or to a cloud facility for making computerized evaluations, can result in improved outcomes by getting people to the emergency room when needed and by giving them the peace of mind to stay home when no intervention is called for.

Similarly, for athletes the sensor patch 1600 can be configured to evaluate an athlete's response to training using cardiac output as a parameter. A sensor patch 1600 with force sensors also may be adapted to monitor breathing rate and lung vital volume, factors of interest to pulmonologists and for athletes in training. An inexpensive disposable version of the sensor patch 1600 that an athlete can use to record and to store key data after a workout offers not only improved individual training, but also can be used to compare training regimens across large groups, an application of big data made possible by easy access to physiological measurements.

The sensor patch 1600 could facilitate further learning about real-time hematological indicia as well. For example, the science of blood oximetry is little studied in the general population. By assembling large cohorts of individuals and obtaining periodic measurements of blood oxygenation using a suitably configured sensor patch 1600 with a simple photo-oximeter sensor, epidemiological studies of air quality, clinical studies of exposure to toxic pollutants, chronic occupational conditions, correlations with age and underlying conditions, and so forth, large volumes of data can be accumulated. Related big-data studies can be undertaken for diabetics and by looking at other blood markers, a broad range of human conditions in health and disease.

The devices and systems disclosed here offer significant advances in telemedicine that may reduce costs of medical care while improving outcomes and providing safer environments and working conditions. When the device 1600 is combined with the network system 2100, the capacity to continuously monitor patient health is combined with the capacity locate the patient, as will be described below in Example II.

Still referring to FIG. 22, alternate embodiments of the sensor patch 1600 are contemplated. For example, the sensor patch 1600 can have an NFC/RFID tag architecture that includes the integrated controller circuit 1626 and the integrated communications circuit including the demodulator 1620 and the modulator 1622, which interface with the NFC/RFID antenna 1601. The communication circuit 1604 and the controller circuit 1626 can be on separate chips or on a same chip. Or, the one or more integrated circuits can be flexible chips such as supplied by American Semiconductor, Inc., of Boise, Id.

The LAN radio 1680 may be a Bluetooth radioset, in one embodiment. Other suitable radioset protocols may include Zigbee, Thread, and WiFi, while not limited thereto. Bluetooth radio is notable for its robust resistance to interference and dropped connections, and has been widely adopted. The BTLE radio protocol standard is more attractive than BT Classic and BTDM protocols because of its low energy consumption. Advantageously, processors with integrated BT radio cores operating at 1.8V are readily available. A BT radioset in standby “always listening” mode may burn less than 30 uAh while retaining the capacity to wake up the processor and accessory circuitry from deep sleep in response to a radio command from a smartphone or a hub or in response to sensor data, and thus supports portable applications for IoT use. A baseline energy budget for an “always on” Bluetooth radioset may consume about 30 uAh assuming an intermittent transmit period of 20 ms, a transmit cycle of 2.5 sec, (i.e., 1440 transmits per hour), and a transmit power of 3.5 mA. Transmit power and frequency may be configured according to the application, and with increasing miniaturization of chip architecture to 14, 10 or even 5 nm gate structures, total energy consumption continues to fall sharply, enabling increasingly longlasting IoT devices in packages using either disposable or rechargeable batteries.

In one embodiment, using a small solar cell associated with a device radiotag, the current needed to maintain the Bluetooth radioset for intermittent transmission of sensor data can be met from the solar cell output. In other embodiments, triboelectric structures that harness kinetic movement to generate current sufficient to support an always-listening radio are realized experimentally, demonstrating that the devices of the invention are well positioned to find increasing number of applications for future IoT needs.

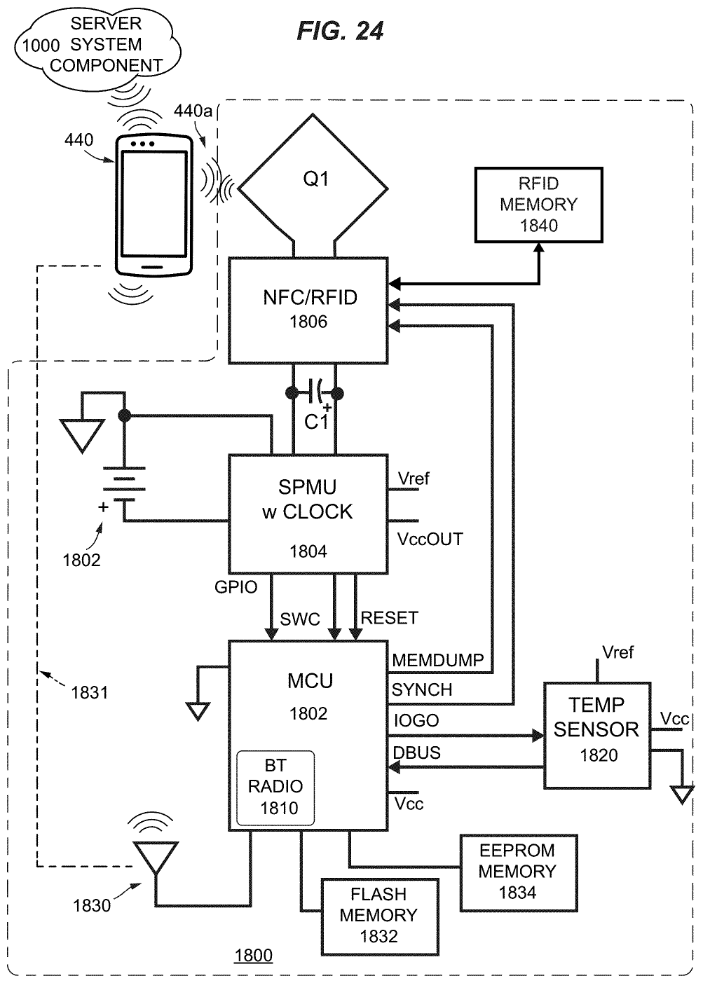

FIG. 23 is a system context view 2300 with schematic of a circuit device 1750 with microcontroller (MCU, 1752), power management unit (SMPU, 1754) having dual power supplies that include an integrated intermittent power supply from NFC/RFID coil Q1, and an onboard disconnectably connectable battery power supply 1766. Vcc is supplied by the switching regulator of SPMU 1754 to the MCU 1752 (MCU), to a sensor package 1759 with ADC, and to other logic components such as flash memory 1760. In some instances, the MCU includes cache memory sufficient for some data logging and firmware for basic processing, but for some data logging applications, a larger memory capacity is required.

The circuit device 1750 includes dual radiosets, an NFC/RFID radioset 1758 with antenna Q1 used for intimate close range secure communications, and a Bluetooth radioset 1756 with antenna 1757 used with encryption for LAN radio connections. The antenna 1757 for the Bluetooth transceiver 1756 can be printed on the circuitboard or can be supplied as an integrated antenna package, for example. By supplying 3 to 20 mA power to a well configured antenna, Bluetooth radio signals propagate from a hundred meters up to a kilometer in range. Thus the Bluetooth LAN radio can complement the very intimate short range NFC/RFID radioset by providing connectivity to remote devices. Relays may also be used to extend range.

In one embodiment, the circuit device 1750 is a disposable part of a larger system that includes a cloud host server 1000, optional VPG 1000a, and intermediary communications device 440. The cloud host server includes an administrative server 1002 for collecting and analyzing data from the circuit devices 1750. The cloud host may also include a REST API for example, that is responsible for interfacing a variety of devices to software services provided by the cloud. And will include a network engine 1004 for handling communications to and from communications devices 440 and other remote devices and terminals such as public health facilities, private medical clinics, home users, and so forth. The cloud host may also have access to a variety of databases useful in assessing epidemiological patterns in a community and in accessing best practices in medical care, for example.

Bluetooth BTLE radio cores are unique in providing an “always on” radio functionality at low power. For example, a BTLE radioset set up to transmit at 3.5 mA can be configured to consume 30 uAh in use by setting a transmit-receive cycle at 2.5 second intervals and a transmit-receive period of 20 ms. This provides 1440 contact opportunities per hour and offers reasonably low latency in the network. BTLE inquiry and page modes, using native access codes, permit selective listening and responsiveness, providing further opportunities to manage power consumption with a robust level of error correction and resistance to interference. BTLE radiosets organize themselves into piconets and include an “always listening” standby mode, with “sniff” and “park” states for reduction in power consumption by peripheral devices.

Thus a combination of NFC/RFID and Bluetooth radiosets offers an attractive combination when interfaced with the global WAN of the Internet. Two approaches for privacy in medical telecommunication of data are possible. In a first instance, a private virtual gateway 1000a is introduced. This is a cloud portal having a private high-level IP Address set up so that only authorized traffic is allowed in a restricted domain that is outside the world-wide web but is organized by HTTP: protocols on a root directory. Devices using VPG technology do not experience the continual radio clutter that bombards public internet connections. Alternatively, the circuit devices 1750 may include an RFID memory cache that stores a TID (Tag Identifier) and a private key or keys. These keys may be communicated to an intermediary smartphone over a nearfield radio link and are essentially impossible to capture by eavesdropping. By transmitting the TID to a network over a broadband communication link in a packet data environment, a dedicated cloud host administrator can look up the key(s) on a secure server associated with the TID by the manufacturer, and without ever broadcasting the keys, can initiate encrypted communication with the circuit device 1750 and smartphone 440—on Bluetooth radio. This strategy overcomes the well known susceptibility of native Bluetooth encryption to interception and decryption. 128-bit private keys may be implemented in this way that can strongly resist efforts to intercept and decode the transmissions, ensuring a higher quality of security for medical data. Users of the circuit devices 1750 are provided with user profiles registered on the cloud service that are extensions of their smart devices, and which give access to a range of services including diagnostic and medical advice, access to chat services with medical professionals, and automated synchronization of sensor data with private physicians engaged by the user, for example.

Employers may also use the cloud services to provide a safer work environment in which fever cases are rapidly identified and referred for follow-up with medical professionals in a confidential system that is HIPPA compliant.

The system administrative server 1000 has access to databases 2116 where user profiles may be stored, and also to public health databases accessible to the community. The system server is able to detect abnormal trends in received temperature data and can respond by making an intervention, including notifying a healthcare provider, waking a parent or family member, a neighbor, and so forth. A chat feature can be supported so that advice and consultation are available through the smartphone and the temperature data is automatically shared with the healthcare provider(s). For example, if a temperature rise is detected in two consecutive temperature readings, the system can autonomously schedule added, more frequent readings, and can generate notifications if warranted to summon help. Similarly, if two or more consecutive temperature readings show a drop in temperature, the system can issue a comforting notification. And if the patch sensor becomes detached, and begins reading ambient temperature instead of body temperature, the system can issue an alert to an attendant.

For temperature sensors that are not disposable, the system will monitor temperature and if a consistent ambient temperature is detected, the system will notify the user that it is returning the sensor circuit to battery shutoff mode to conserve power, such as would be appropriate when a user has no further need for the device and has returned it to the bathroom medicine cabinet. Thus having an internal soft switch for controllably disconnecting and connecting battery power provides a convenient way to reduce battery power losses during storage and extend the usable lifetime of the device between charges or battery replacements. The softswitch is connected to battery using the auxiliary power of a near-field source signal as described above, but battery shutdown and cold storage can be accomplished with either the near-field or the Bluetooth radiosets.

Smartphone 440 is representative of a family of “hub” devices. With suitable software and APIs, a smartphone or hub can interface with cloud server 1000 to perform a variety of “software as a service” functions that support temperature monitoring and logging by device 1750 in a network environment. Most smartphones include both NFC/RFID and Bluetooth transceivers suitable for use in collecting data and providing commands to the sensor patch device 1750. While hub devices have long been known, recent introduction of the Echo, Dot and Alexa hub devices with voice interface have increased their popularity. Moreover, embodiments of hub 3606 described in conjunction with FIG. 21 and LAN radio 1680 described in conjunction with FIG. 22 may be applicable to the network 2300 of FIG. 23.

While the MCU may also include a Bluetooth radioset (1680, FIG. 22), as is readily available as an integrated package, FIG. 23 shows an SPMU 1754 with integrated Bluetooth and NFC/RFID radiosets. This combination is novel and has synergy in combining an always on oscillator or real-time clock with an always on radioset that functions to wake up the MCU when needed and also serves as a switching regulator for directing power from the coil Q1 or from the battery 1760. Modulation of the near-field radio transmissions and modulation of frequency used in digital Bluetooth radio transmissions are integrated in a single SOC. For data logging, these devices are organized into an “always on” core that includes at least one internal clock and the core Bluetooth radioset. In this build, the core of the BTLE radioset controls the sleep/standby status of other components of the device and responds to BT radio traffic, sensor data, or interrupts from the NFC/RFID radio.

By including another core that controls and monitors sensor output at low power, the device can further respond to excursions of the sensor data output that depart from defined normal ranges, providing an added wake up function for the BT radio as housed in the SPMU 1754. In this view the MCU 1752 is primarily responsible for basic housekeeping, encryption and memory functions, and can be build as a low cost/low power microcontroller operated using firmware.

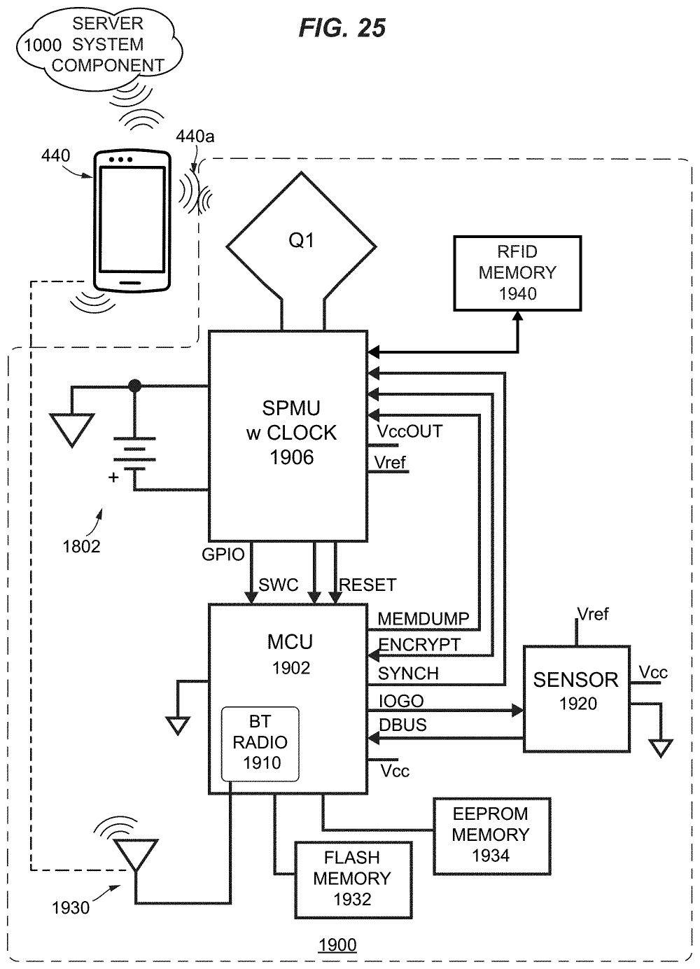

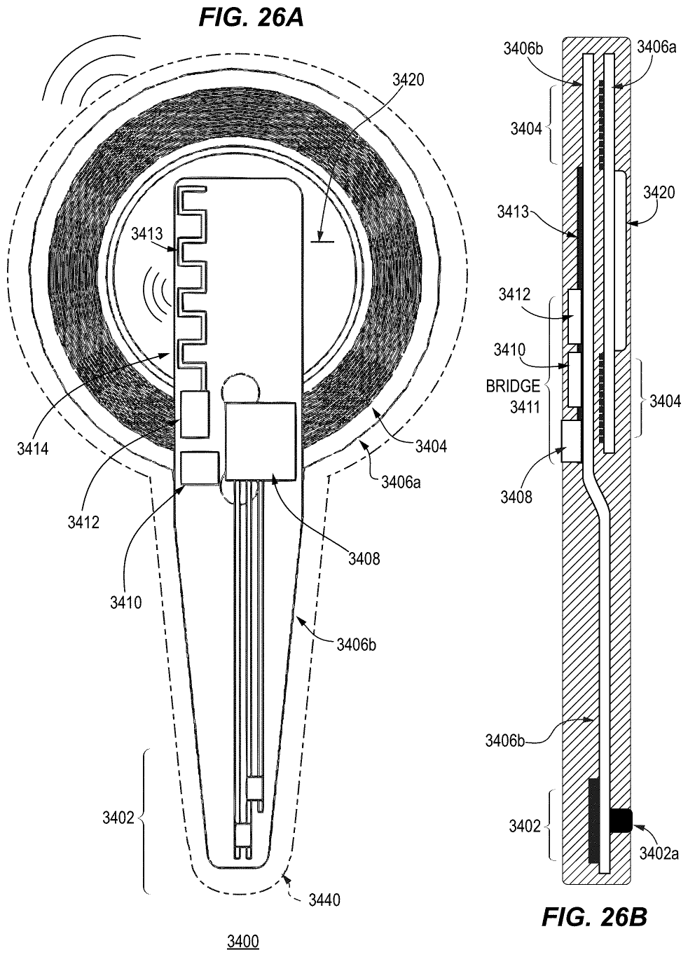

As currently available, many SOC chipsets include a NFC/RFID core with PMU for switching between near-field and battery power and a microcontroller in an integrated package. A BTLE radioset is a separate component or is supplied as part of a separate microprocessor. Details related to the power supply management of dual powered, dual radio devices of this kind are described with reference to FIGS. 24 and 25.

FIG. 24 is a representative schematic circuit diagram of a temperature logging patch 1800 that is microprocessor-based and operates with a smartphone 440 using NFC/RFID radio or a Bluetooth radio to transmit and receive data. In a typical system, the Bluetooth radio core 1810,1910 is integrated into the microprocessor 1802,1902. The battery 1802 is a 3V coin cell built into the data logging device. Referring to FIG. 24, the SPMU 1804 is an LTC3330 power management ASIC (Linear Technology, Milpitas Calif.) that includes buck-boost regulators, a rectifier, and logic circuitry for managing power to other circuit components. The LTC3330 functions as a switching regulator that can accept power from one or more outside energy sources, selecting the power supply at any given time that is optimal either by use of a low-drop out regulator or by logic selection according to the value of an IO pin. The chip also supplies flag signals that can be used to program other logic components and contains MOSFET switches sufficient to turn on and off the battery voltage according to program instructions. As used here, the battery circuit is supplied prior to end use as an open circuit through SPMU 1804, but a FET switch in the SPMU is designed to be closed in an boot initiation protocol that is executed when power and data are supplied from a qualified NFC/RFID field 440a acting on NFC/RFID chip 1806 (analogously to the circuits described in earlier sections). The NFC/RFID chip can be a MAX66242 (MAXIM Integrated, San Jose Calif.), for example. Power from smartphone 440 turns on NFC/RFID unit 1806, which supplies sufficient voltage to power up the SPMU. The SPMU can then permanently keep itself on by closing a switch to battery power. By placing a real time clock and system clock in the SPMU, the MCU can enter a sleep mode when not in use and can be woken up by a timer operated in the SPMU. The real time clock, whether implemented in the SPMU 1804 or in the MCU 1802 (under battery power) can be operated to log timestamped data by intermittent execution of an instruction cycle that actuates temperature sensor 1820 even when NFC/RFID power is not present. Generally the real time clock will count from an initialization time and system clocks will count down for pre-determined intervals. By integrating control of these clocks with a user interface on a companion smart device 440, data can be timestamped for delivery to the smartphone and measurement cycles can be scheduled for automatic execution when the user is not in attendance. The smartphone, in response to received data, can construct a table showing the chronology of temperatures and can display the chronology as a plot. Or these higher functions can be done by the cloud host 1000 and delivered to the smartphone for display.

In alternate embodiments, the MCU can be an EMF32ZG110F32 Zero Gecko microcontroller from Silicon Labs (Austin Tex.), which is a very low power controller with capacity to execute instructions on a duty cycle operated with an onboard clock over hours or weeks. In a second embodiment, the MCU can be an STM32L011x4 having ARM®-based Cortex®-MO+ and 16 KB Flash, 2 KB SRAM, 512B EEPROM and onboard ADC sensor signal processing for data logging (STM, Plan-les-Ouates CH). In yet another an alternate embodiment, the SPMU and NFC/RFID radioset with rectifier can be integrated in a single package such as the NHS3100 temperature logger (NXP Semiconductors, Eindhoven, Netherlands). In yet another embodiment, the MCU may be a NXP NHS3100W8 Temperature Logger (NXP Semiconductors, Eindhoven, Netherlands) with NFC/RFID interface, 44 kB memory including 32 kB flash memory and 4 kB EEPROM, and a Zn/MnO2 battery providing 20 mA at 3.0V.

In some embodiments, the MCU includes an integrated Bluetooth radioset 1810, and can communicate wirelessly with smartphone 440 via antenna 1830 and Bluetooth link (1831, dashed line). In other embodiments, the MCU does not include a BT radioset, which is provided separately or as an integrated SoC in a custom ASIC.

The temperature sensor 1820 may be a thermistor package and may include an integrated ADC or may be a digital temperature sensor. For example, an STTS22H digital temperature sensor is slaved to an I²C bus for data acquisition and low power consumption in a first embodiment. In a second embodiment, the temperature sensor may be a MAX31785 with low error in the range of 25 to 50 C.

Circuit devices 1800 of this kind can be formed as wearable patches for measuring skin temperature or as insertable thermometers for measuring sublingual temperature, for example. In some instances a pair of sensors S1, S2 may be used when measuring skin temperature as described with reference to FIG. 17A.

Advantageously, the device can be actuated and initialized using a smartphone transmitting power and data via coil Q1. The data can include configuration parameters sufficient to initialize the microprocessor and to form a closed circuit that starts battery power to the processor and SPMU 1804. By storing the battery 1802 as an open circuit, and closing the battery circuit connection only upon first use, battery shelf life can be significantly extended for the consumer. In some instances, it is desirable to maintain battery power to the MCU and memory for the duration of any needed data logging application. In other instances, it may be desirable to interrupt the battery circuit when the consumer places the device in extended storage, for example a household digital thermometer that gets placed in a medicine cabinet when not needed. The NFC/RFID chip 1806 may work in cooperation with the SPMU 1804 to manage the battery circuit, opening or closing a battery power switch. MEMDUMP allows the user to access the history of temperature readings even if battery power has been disconnected or the battery voltage is insufficient to meet a low dropout threshold specified for operation of the MCU. MEMDUMP is made active by a call from the external reader 440 for an upload of the data stack in memory. The call may be answered by transmitting the available data across the Bluetooth link 1831, or by transferring memory via MEMDUMP to the NFC/RFID modulator for transmission over a near-field communications link on antenna Q1.

Generally, flash memory 1832 and EEPROM 1834 memory are provided to supplement limited cache memory onboard the MCU or SPMU. The SPMU may include a real time clock that is part of an “always on” core when battery powered and may provide one or two system oscillators for interrupts and for countdown wake cycling to perform automated measurement cycles at timed intervals or at times that are programmable by the user. Utilities for user commands issued to the circuit device 1800 are entered on a user interface built in to the client API that operates on the smartphone 440 or hub 3606 (FIG. 21).

The device may also contain RFID memory blocks 1840. For Gen2 RFID tags, these include EPC (96 or more bits for electronic product code), Reserved (64 bits for lock and kill passwords), TID (unique tag ID assigned by manufacturer) and User (512 or more bits). Extended memory is also available in blocks of 4K or 8K bytes, as is sufficient to store basic instructions such as an API command to open a link to a dedicated IP Address or to store a condensed history of use of the tag. Tag formats having memory for extended use include the Omni-ID Adept 850 for example. More detailed tag usage history can be stored in a network administrative database associated with unique tag TID that is accessible through a cloud host server.

Depending on details of the configuration, the Bluetooth radioset may also be powerable via the NFC/RFID coil Q1, and any instruction set provided as an application for installation on companion smart device 440 can include instructions whereby NFC/RFID or Bluetooth are used to access data from the MCU. Thus FIG. 24 shows the companion smartphone 440 in radio connection via both of the antennae, Q1 or the Bluetooth antenna 1830. Radio link 1831 indicates that the Bluetooth radioset 1810 is operated to establish communication with smartphone 440 independently from an NFC/RFID mediated radio connection. The Bluetooth radio connection at 2.45 GHz has a range of several meters to a few hundred meters, or greater at higher power, so as to eliminate the need for near-touching proximity of the NFC radioset at 13.56 MHz.

FIG. 25 is a view of an exemplary circuit 1900 operating as an NFC/RFID and Bluetooth sensor data logger in cooperation with a smart phone 440. Circuit device 1900 includes proprietary encryption and security features for protecting data. The lock access key that may be stored in RFID memory 1940 may also be used to prevent device tampering and to restrict data access to authorized users.

The NFC/RFID radioset 1906 can be a MAX66242 (MAXIM Integrated, San Jose Calif.) operable with 4096 Kbit EEPROM memory and an I²C master/slave interface and integrated NFC/RFID ISO 15693 interface. The MAXIM chip includes features that combine SPMU functions with an integrated NFC/RFID power interface. The MAX66242 also includes an encryption key in memory and can be programmed to authenticate to a system during an initialization and then share encrypted data only with readers having the required key. Because smartphones and hubs 440 function not only as NFC/RFID readers but also as Bluetooth and WiFi radio transceivers, the authentication performed initially by NFC/RFID can be used to secure and encrypt transmissions made across the LAN radio bands as well. In one instance, a private key is communicated on the nearfield communications link and is used subsequently on the LAN Bluetooth communications link. The private key may be 128 bit encryption for medical quality data protection, for example, and may be stored in RFID memory 1940 and shared only with the smartphone via the private NFC/RFID short range transmission. A private key that has been placed in memory within device 1900 will be known to the manufacture by association with a tag identifier (TID) unique to the patch sensor radiotag and can be retrieved from a secure administrative database to decode data transmissions received by a cloud host server. Any application installed on the smartphone can be made HIPPA compliant so as to secure the system uplink to higher level secure servers. By virtue of the inherent intimacy of the NFC/RFID radio link, which operates only with a smart device 440 within inches of the coil, the security of Bluetooth broadcasts can also be encrypted for subsequent data and command radio exchanges.

In another embodiment, power management can include a MOSFET switch in the SPMU 1906 for example that turns on and off V_{ccOUT} power to the device in response to an encrypted signal from any authorized Bluetooth or NFC/RFID communications device. The setup of circuit device 1900 requires that an authorized smartphone 440 or other smart device be placed immediately adjacent to the device and that an NFC/RFID authentication and initialization protocol be executed. Generally this is achieved by installing an “app” in the smart device that recognizes patch sensors having the required NFC/RFID radioset, sending an authentication signal to the patch so as to initialize the patch for use. First boot of MCU 1902 causes the patch to switch to battery-powered “data logging” mode with intermittent power to the MCU and sensor package (under control of a clock in the SPMU 1906), and enables data exchange over BTLE radio 1910. If the battery power fails or is somehow disconnected, onboard memory stack can still be retrieved using an NFC/RFID reader such as by near-field link 440a with smartphone 440.

Generally, flash memory 1932 and EEPROM 1934 memory are provided to supplement cache memory onboard the MCU or SPMU. The SPMU may include a real time clock that is part of an “always on” core when battery powered and may provide one or two system oscillators for interrupts and for countdown wake cycling to perform automated measurement cycles at timed intervals or at times that are programmable by the user. Utilities for user commands issued to the circuit device 1900 are entered on a user interface built in to the client API that operates on the smartphone 440 or hub.

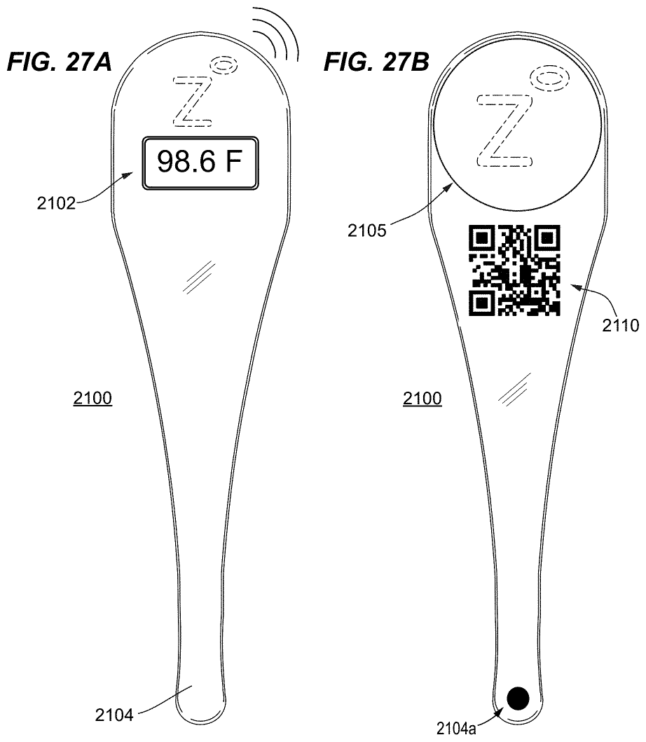

Circuit device 1900 can be made in a variety of packages for measuring a clinical condition of a patient, and may have device/apparatus form factors of a patch, a strip, a spot, or a thermometer sublingual package as shown in FIG. 26A, for example, while not limited thereto, and may include other sensors.

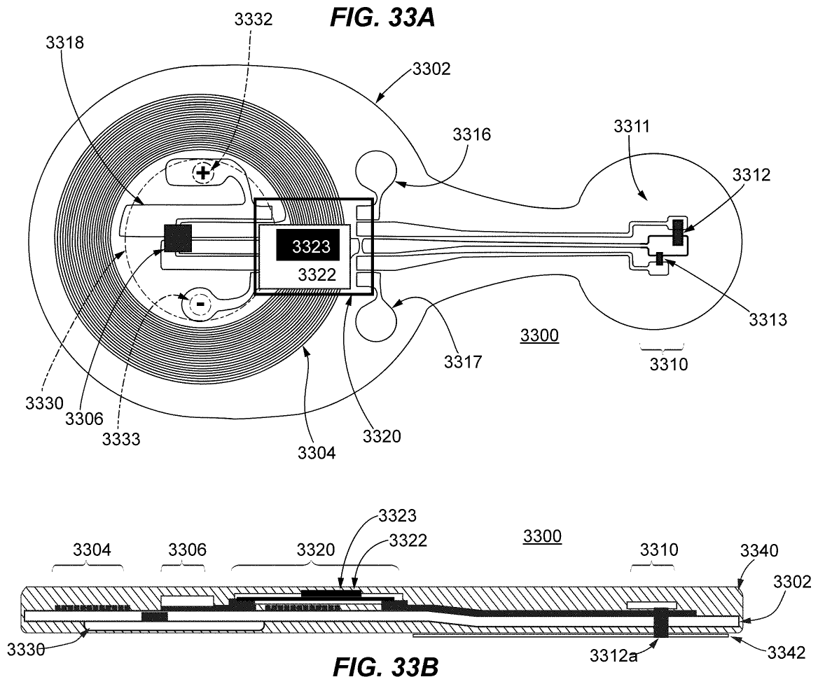

Alternatively an SOC approach can be taken in which an entire power, sensor and comms system with NFC/RFID and LAN radiosets and memory is incorporated in a single microprocessor chip, and the board includes the NFC/RFID coil Q1 and the Bluetooth antenna 1830 in a compact footprint. In other instances the SOC may include processor, Bluetooth radio, power management and memory as an integrated chip mounted on a staple circuit board, such as shown in FIG. 33A, in which an NFC/RFID coil with isolated temperature sensor assembly is connected in a second assembly step. The NFC/RFID circuit component may also include a pre-printed battery constructed as shown in FIG. 33B.

FIG. 26A is a open schematic of an NFC/RFID sublingual thermometer 3400 with Bluetooth® radio antenna 3414 and NFC/RFID coil 3404 in a package 3440, according to an embodiment. The NFC/RFID coil is formed on a first flexible substrate 3406a. A print-on battery is applied on the reverse face of the first flexible substrate 3406a and packaged in an encapsulating sheath or layer 3440. Vias connect the battery and the inductive coil to a power management unit 3410. FIG. 26B shows the device 3400 in a simplified cross-section.

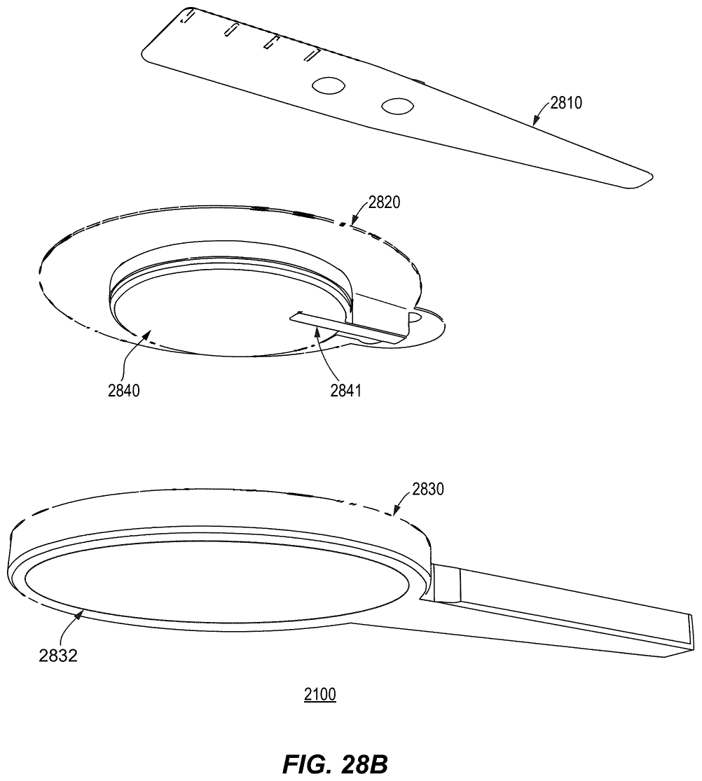

The thermometer 3400 includes a thermistor 3402 with probe 3402a mounted on an extended nose of the flexible substrate layer 3406b. Three wires extend from the thermistor 3402 to a microcontroller (MCU) 3408. The thermistor may include a sensor probe that extends from any substrate baselayer so as to be in direct content with the skin or surface in need of temperature monitoring and may be surrounded by a compatible adhesive to ensure good thermal contact. Logic circuitry can be, or can include, a microcontroller or microprocessor, or can be, or can be included in, an application-specific integrated circuit (ASIC). Possibly separate components include an NFC/RFID power management unit (SPMU, 3410), a Bluetooth® radio controller 3412 with antenna 3413. NFC/RFID coil 3404 is disposed on a separate substrate layer 3406a and is transferred to the circuitry on a substrate layer 3406b through two vias, one inside the coil and one outside the coil. To connect to these two vias, the PCB 3406b bridges the coil 3404 at bridge section 3411 (bracketed, FIG. 26B). By printing, or otherwise forming, the coil 3404 and the other circuit component circuitry on separate PCB layers 3406a and 3406b, the backing of PCB 3406b insulates the component circuit traces from the traces of the coil. After both subcircuit subassemblies 3406a, 3406b are qualified by testing, the joining vias (not shown) are joined to their pads by soldering or using conductive adhesive after alignment of the two layers; this ensures a higher qualification success rate of completed devices because only subassemblies that have passed first quality control checks are bonded as a sandwich and tested again. The concept of the coil PCB 406a as a separate board/component is described below in more detail in conjunction with FIGS. 28A, 28B.

Battery 3420 may be printed on the reverse face of PCB 3406a, or otherwise may be disposed on the PCB 3406a and held in place with one or more conventional battery connectors. Alignment of the vias ensures connectivity of the battery and the coil to the power management chip 3410, which includes a rectifier and other circuits for output of a controlled Vcc to other circuit components. Flexible substrates, from which one or both of the PCBs 3406a and 3406b can be made, include thin films of polyethylene terephthalate, polyamide, and polydimethylsilane, for example. The package 3440 can be a sealed casing, or can include an access port for replacement of the battery, for example. Coin cell or NiMH batteries may be used for example, and the battery may be inductively rechargeable, if desired, to relieve the need for opening the case. The dimensions of the case may be configured to as to permit its use in measuring sublingual body temperature of a subject or for use in other body orifices. Alternatively, the case may encapsulate the circuit such that the entire device may be seated under the tongue and read transdermally by radio. And in yet other embodiments, the case may be a sealed implantable capsule or chip for subdermal use, and include RFID information that is useful for identifying an animal that carries the chip. In this instance, probe sensor surface 3402a may be enclosed in the sealed capsule because the entire device will be at thermal equilibrium with the surrounding tissue. Capsules that can pass through the gastrointestinal system while transmitting sensor information are also contemplated.

SPMU 3410 may be a switching regulator, and optionally may be integrated with the MCU or with the NFC/RFID radioset. It includes an internal clock or clocks and once activated in an NFC/RFID source signal directed from a smart device, switches on battery power to other parts of the circuit and defines standby and sleep states for components such that an “always on” core in the SPMU maintains clocks and counters for scheduling component wakeup as needed and for timestamping data. The system clock may be initialized by data in the NFC/RFID source signal or may be initialized when the Bluetooth radioset is engaged at battery power startup. A low energy oscillator in the Bluetooth core may also be used to time wake up sessions for the Bluetooth radio, and the Bluetooth core may include a standby mode in which frequent listening and advertising sessions are powered so as to keep in frequent contact with any external smartphones or hubs with minimal downtime latency.

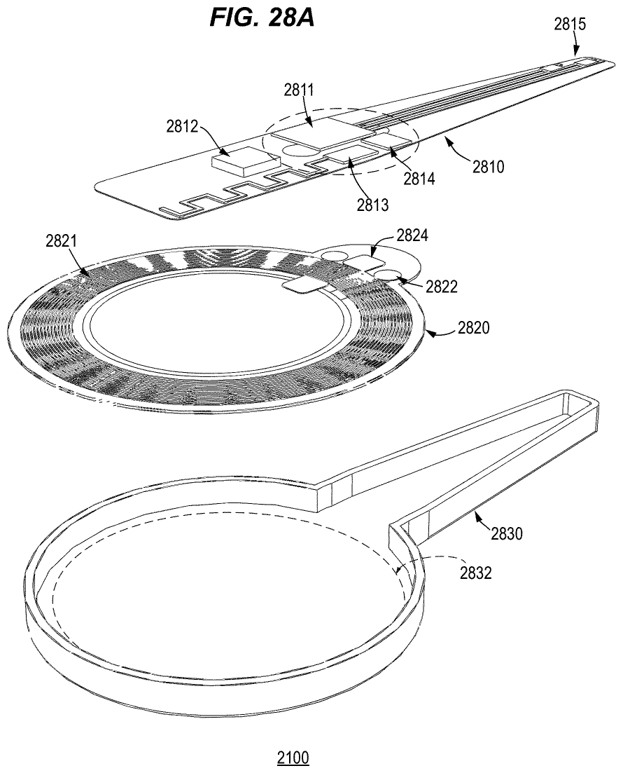

FIGS. 27A and 27B are views of a sublingual thermometer package 2100 including a circuit board with microcontroller, Bluetooth radioset, switching regulator, and NFC/RFID antenna and radio circuit, in which the switching regulator is a low dropout regulator configured to supply power from the NFC/RFID radoset or from a replaceable battery 2840 as shown in FIG. 28B. In this example, the circuit board is shown with an enclosing housing for use as a sublingual thermometer device. The device is housed in a plastic “clamshell” case that may be ultrasonically fused to be waterproof.

The nose 2104 houses the temperature sensor, which is inserted sublingually in use. The nose may include a metal implant 2104a to improve heat conductivity between the outside of the case and the temperature sensor.

FIG. 27A is a view of a device 2100 of the kind introduced in FIG. 26A, but includes a clamshell case with LCD display 2102 mounted on the top cover of the case for providing temperature readouts locally as well as via radio.

FIG. 27B is a view of the backside of the device 2100, with removable circular battery cover 2105 for replacing a coin cell battery inside. The back wall also includes a QR Code 2110 that encodes an IP Address, which when scanned by a smartphone, directs the user's browser to a website for installing a smart device compatible software application and pairing with the user's smartphone analogously to the systems of FIGS. 23 and 24. A cloud host server 1000 plays the role of server system component and the smartphone the role of client system component to complete setup of the device prior to first use. Through the website link, the cloud host will cause the appropriate “app” to be downloaded and installed into the smartphone.

The initialization process may occur in a series of steps. After installation of the “app” needed to operate the device, the user will be invited to “open” the “app”, whereup instructions in the app causes the smartphone to radiate near-field radio energy directed to the device 2100, which is held close to the smartphone. The device goes through an initial boot and switches ON its battery powered SPMU and MCU as described earlier. The NFC/RFID radio field may also read unique device identifiers such as the TID, UUID or private keys from the device. Under battery power, the device then will pair over BTLE radio with the smartphone for efficient exchange of data at a distance. The user may then employ the device as a thermometer while placing the smartphone on a desk, for example. The smartphone will display a user guide and one or two screens for setting up a user profile so that temperature measurements can be stored in a file created for the patient in need of temperature monitoring. Each time a temperature reading is made, it will stored in memory and also be displayed on a paired smartphone. Over time, temperature data can be plotted to show a trend.

FIGS. 28A and 28B are exploded views of a dual-radio thermometer package 2100 with more internal detail. The package is analogous to that of FIG. 26A but includes a replaceable coin-cell battery 2840 instead of a print-on battery 3420 and also includes a speaker or LCD display 2812 as part of the enhanced user interface depicted in FIG. 27A.

The sublingual device includes a microcontroller 2811, Bluetooth radioset 2813, switching regulator 2814 mounted on a first substrate 2810. The switching regulator is a low dropout regulator configured to supply power from the NFC/RFID radoset or from a replaceable battery. The thermistor 2815 is thermally isolated on a nose of the substrate and may be provided with a thermally conductive member that extends through the case.

A second substrate 2820 supports an NFC/RFID antenna and radio circuit. A battery is supported on the underside of the second substrate. Power and data are coupled by conductive pads the connect the first and second substrates. The microprocessor 2811 on the first substrate is part of a bridge that connects the inner and outer pads 2824 of the NFC coil 2821.

Only the bottom part of the clamshell housing 2830 is shown. The upper panel, including any display window is sonically welded in place after insertion of the circuitry. An access port 2832 may be provided to facilitate exchange of the coin cell battery 2840.

Coin-cell battery 2840 is disposed on the bottom face of substrate 2820 and is in electrical connection with the circuitry of both the first and second substrate members 2810,2820. A clip 2841 holds the battery in place and serves as an electrode to complete the circuit.

FIGS. 29A, 29B, 29C, 29D, 29E and 29F are design views of a sublingual dual radio device with switching power regulator.

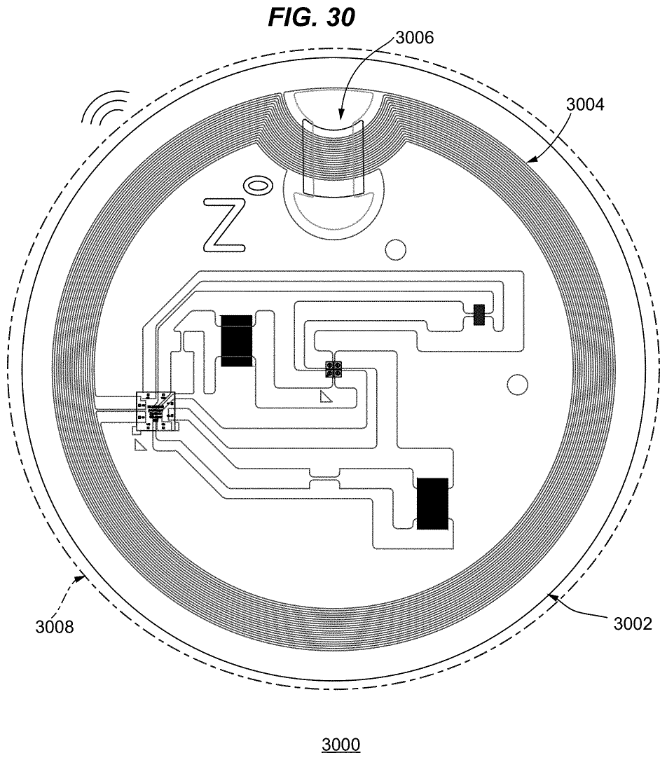

FIG. 30 is a schematic of a sensor patch thermometer 3000, according to an embodiment. The thermometer 3000 includes a printed circuit 3002 powered via a printed bridge 3006 by a coil 3004. The circuit 3002 includes a rectifier and a power-supply bypass capacitor for generating a stable DC voltage from an NFC/RFID field or signal from a remote active NFC/RFID device, pull-up resistors, and logic circuitry for determining a temperature sensed by a temperature sensor. Generally the circuit 3002 is formed on a flexible substrate by a process of etching a conductive layer and soldering or adhering (e.g., with a conductive glue) circuit components to the resulting circuit pads. The NFC/RFID field or signal supplies not only power, but also can be modulated, in a conventional manner, using on-thermometer components to exchange digital data with an the reader or other device generating the NFC/RFID signal. However, data is typically read passively. That is, the thermometer 30 uses power from the NFC/RFID signal to modulate the signal (e.g., by amplitude shift keying (ASK) using load modulation) with the determined temperature and other information (e.g., identify of the person wearing the patch sensor and whose body temperature was determined), and the NFC/RFID device generating the signal (e.g., a smart device such as a smart phone or other NFC/RFID reader) demodulates the signal to recover the determined temperature, and, if included, the other information.

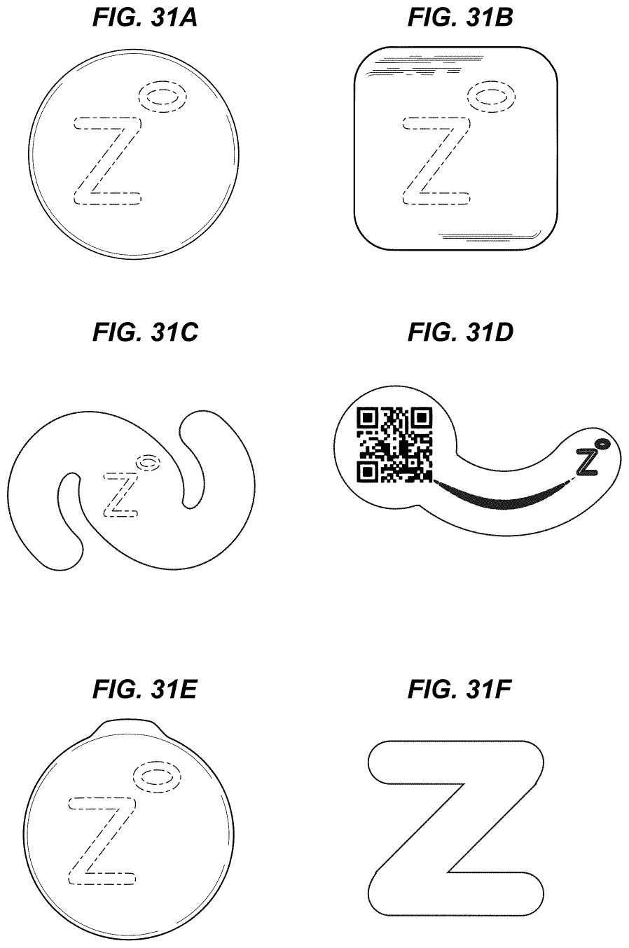

FIGS. 31A, 31B, 31C, 31D, 31E and 31F are designs of sensor patch thermometers of the kind depicted schematically in FIG. 30. Designs 31C, 31D and 31F are adaptable to contoured surfaces of skin where the topology is curved in multiple planes. Design 31D includes a QR code as can be useful in improving the user experience by providing direct access via a smartphone camera to the needed software and administrative cloud services.

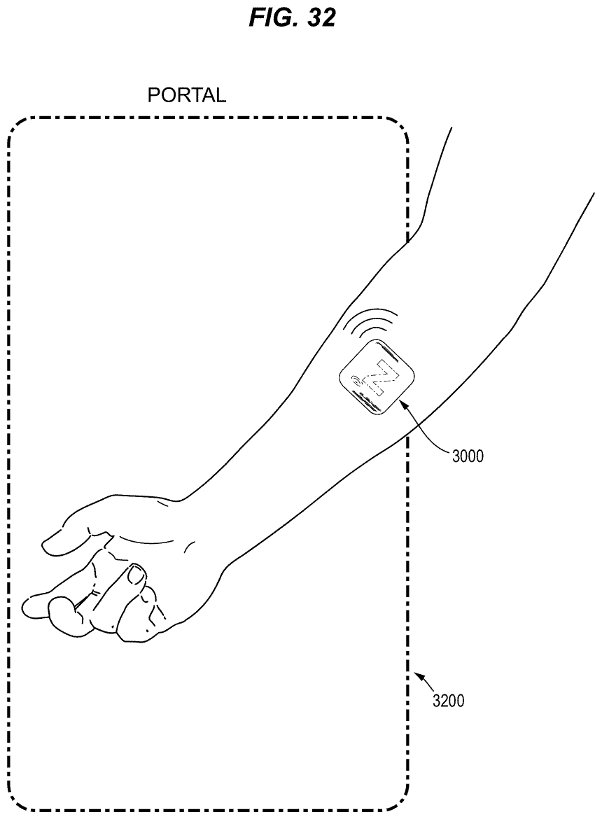

As shown in FIG. 32, the sensor patches 3000 may also be used with an NFC portal 3200 rather than a smartphone, as in an entrance to an airport or workplace, for example. Subjects wearing the patch are monitored as they pass through the portal and any subjects with an elevated temperature may be identified for further screening.

FIG. 33A is a plan view of a thermometric skin sensor patch 3300, according to an embodiment. FIG. 33B is a side view that exposes internal sections of the device body. The patch sensor 3300 includes printed circuitry for determining a temperature sensed by a thermometric sensor 3310, which is formed here as a thermistor 3311 with calibrated resistor 3312 and reference resistor 3313 pair. Two substrate layers are shown, the first a base substrate layer 3302 that supports an NFC/RFID coil 3304, a power management system (SPMU, 3306), a battery 3330 and a thermistor 3310; the second substrate layer 3320 is a “staple” that supports a bridge circuit with processor 3322 with BT radioset core 3323 and antenna (not shown). In essence, the staple 3320 serves the function of the printed bridge shown in earlier figures (FIG. 5B). The calibrated resistor 3312 is disposed in a performation in the abase substrate layer 3302 such that the sensor surface 3312a contacts the underlying skin of a subject when adhered to a body. The body of the resistors of the thermistor assembly 3311 may be engineered so that the thermal conductivity in the solid is known and reproducible.

Generally the circuitry is formed by a process of etching a conductive layer and soldering or adhering (e.g., with a conductive glue) circuit components to the resulting circuit pads. Test circuit pads 3316, 3317, 3318 are provided as an aid for quality control in manufacturing. Pads 3332,3333 support vias that connect to the battery 3330. The print-on battery 3330 is formed on the underside of the flexible substrate 3302, and may have a non-rechargeable Zn—MnO chemistry, for example. The battery is connected to the circuitry by vias perforated through the substrate (marked here + and −).

The size of the battery is determined by estimating the required duty cycle life. A first estimate can be made by estimating the Bluetooth radio power consumption in active receive mode because the Bluetooth core can be used to control the energy supplied to other circuit components, awakening them as needed. A baseline energy budget for an always responsive Bluetooth radioset is about 30 uAh assuming a transmit period of 20 ms, a transmit cycle of 2.5 sec, (1440 transmits per hour), and a transmit power of 3.5 mA. Thus a 45 mAh battery would be expected to have a continuous service life of 60 days.

The device is generally encapsulated in one or more sealant coatings or layers 3340. The device may receive an adhesive layer 3342 applied to at least a part of the sealant capsule, generally on the underside beneath the flexible substrate layer 3302 where the sensor 3312 must be in thermal contact with the skin.

Flexible substrates, from which one or both of the substrate layers 3302,3320 can be made, include thin films of polyethylene terephthalate, polyamide, and polydimethylsilane, for example. Overlays can include woven fabrics embedded in a silane matrix for better breathability. Any underside adhesive is a non-irritating adhesive as know in the art.

The NFC/RFID field or signal supplies not only power, but also can be modulated, in a conventional manner, using on-thermometer components to exchange digital data with a smartphone, NFC/RFID reader or other device generating the NFC/RFID signal. Exchange of commands and data with the patch sensor tags is passive. That is, the patch sensor 3300 uses power from the NFC/RFID source signal to modulate the signal (e.g., by amplitude shift keying (ASK) using load modulation) to encode and transmit the determined temperature and perhaps other information (e.g., a Tag Unique Identifier, TID) of the patch. The NFC/RFID device generating the signal (e.g., a smart device such as a smart phone or other NFC/RFID reader) demodulates the signal to recover the determined temperature, and, if included, the other information.

The Bluetooth BTLE radioset in one embodiment is supplied as an integrated core within the processor. The process, radio and antenna are fabricated on a separate substrate and are joined to the coil and thermistor circuitry by vias (not shown). The lead line routing is configured so that a switching regulator in the SPMU 3306 can accept power from either the NFC/RFID coil (when irradiated by a compatible NFC/RFID field) or from battery 3330. Various levels of SOC integration can be implemented.

In one embodiment, a first use of a patch is initiated by directing an NFC/RFID radio source field of a smartphone toward the patch. The field induces a current in the coil 3304 which is rectified and powers up the SPMU 3306, which in turn starts its internal clock and powers the processor 3322. The initial data exchange is by modulation and demodulation of the source field. In subsequent initialization, the processor activates the BT radioset 3323. When using a smartphone with a compatible application installed, the initialization and configuration of the circuit to run on battery power is completed by switching an internal switch in the SPMU to connect the battery 3330 to the processor 3322. The processor or the SPMU conditions the power supply (either from the battery or the NFC/RFID coil) and supplies a reference voltage to the thermistor when making a temperature reading. But after the initial power up, battery voltage supplied to the circuit keeps the internal clocks running so that consecutive temperature measurements can be made without operator attendance. Memory is supplied to store temperature readings on board with a timestamp and optionally a geostamp as will be described further below. Generally autonomous temperature readings are scheduled by the app running on the smartphone, but in an embodiment, a host system server in communication with the smartphone takes over active temperature monitoring and schedules temperature readings according to trends in earlier readings and according to epidemiological context based on a community of temperature readings in a geographical area. This fever mapping is also reported to public health authorities with a configurable level of privacy. The Bluetooth radio facilitates this. Once the smartphone has been paired with the sensor patch 300, then the smartphone will continue to monitor temperatures even at a distance of 100 meters or more. A hub may also be provided for measuring temperatures remotely. The temperatures may be reported from the smartphone or hub to a local server or to a cloud server so that charting is automated. The data is always encoded (and may be encrypted) to include an identifier unique to the individual patient (“subject”) wearing the patch so that the system can monitor consecutive temperature readings to detect increasing fever, breaking fever, and so forth, and to issue alerts to parent, physicians, or health professionals if there is a change in the patient's condition.

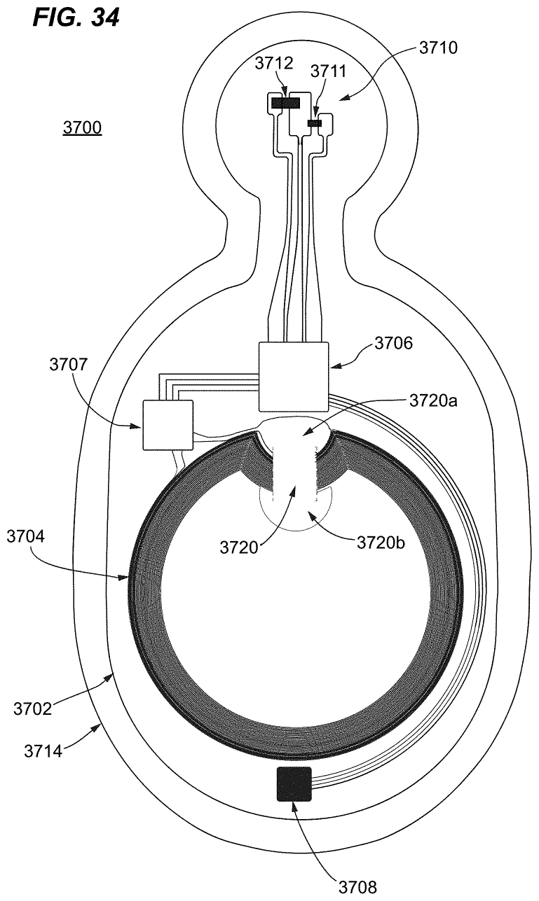

FIG. 34 is a plan view of a sensor patch 3700, which can include circuitry related to the circuitry of the sensor patch 100 of FIG. 1, according to an embodiment.

Here, the sensor patch 3700 measures about 2 centimeters (cm) in width and may be about three to ten centimeters in length. The device may be less than 1 millimeter (mm) in thickness or may be encapsulated in a rigid housing 2 to 6 mm thick. The circuit board 3702 in one embodiment is a flexible printed circuit board or substrate and includes an antenna coil 3704, microcontroller 3606, integrated transceiver and NFC power circuit 3707, an RGB LED 3708, a temperature sensor 3710 with thermistor 3711 and reference resistor 3712 forming a voltage divider, and a cover layer or layers 3714. Other pads may be included for quality assurance testing, for example. One side of the device may be layered with an adhesive. The length can be configured as needed.

The antenna coil 3704 includes conductive loops disposed around a periphery of the flexible substrate. While the resonant loops are shown here to be circular, the closed spiral may follow another shape if desired. The antenna coil is a closed spiral, and includes a crossover or bridge 3720 that crosses from pad 3720a to pad 3720b to complete the resonant loop without “short circuiting” any two or more of the antenna loops together. From the inner pad 3720b, the continuous conductive trace that forms the spiral loops of the antenna coil winds around the periphery of the substrate to the outer node 3720a of the antenna, and the bridge 3720. The bridge 3720 is isolated from the antenna coil loops by a dielectric layer under the bridge. The bridge may be printed with silver ink for example; the dielectric printed layer with dielectric ink. Dielectric inks include SUB, a cationic/thermally cured epoxy insulator and nanoparticle Al₂O₃ inks. An inkjet printing process may be used to form the bridge 3720. Polyimide film may also be used as a dielectric layer to isolate the silver crossover.

A capacitor may be supplied as a low pass filter between the RF coil and the microcontroller. The NFC power chip 3703 may include a capacitor. Power from the antenna coil drives the measurement cycle.

The temperature sensor 3710 is coupled to the microcontroller 3706 by conductive traces formed on the substrate. Thermistor 3711 and reference resistor 3712 form a voltage divider. The temperature sensor may be similar to the temperature sensor 210 of FIG. 2A. A heat conduit may be formed in the substrate under the thermistor and may be air-filled or filled with a solid heat conductor.

In alternate embodiments, the microcontroller 3706 may include a memory circuit with firmware configured so that when powered from a superconductor or onboard battery (not shown, but see FIGS. 26A-26B for details), the device can function as a data logger, reporting a chronology of recorded temperatures when interrogated by a smart device.

LED 3708 is shown here with two leads and is selected for a low power draw with good visibility as a reporter. In other embodiments, a piezo beeper can be substituted for the visual reporter device.

The flexible substrate 3702 is formed from a translucent and flexible plastic such as polyethylene terephthalate (PET), polycarbonate (PC), nylon, a fluoropolymer, polyimide (e.g., KAPTON®), polydimethylsiloxane (PDMS) or other higher dielectric plastic. The substrate 3702 is coated with an aluminum or copper film, and then is masked, etched, and otherwise processed in a conventional manner to form the antenna coil 3704 loops, other conductive traces, conductive pads, and conductive connection points such as pads at 3720a and 3720b that interconnect the components of the circuitry. In this embodiment, the substrate is a single-sided flexible circuit board. Conductive vias may be used to connect a superconductor or battery on the opposite side of the film, for example.

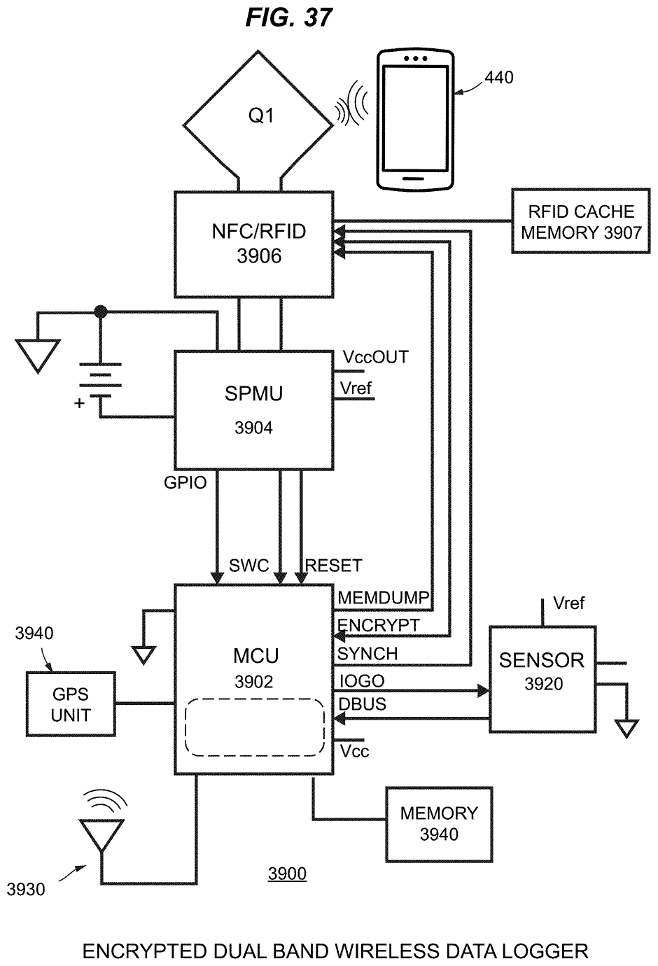

A “chip” side (i.e., the side on which the components are mounted (the chip side is facing up out of the page of FIG. 37) of the substrate 3702 can be sealed from water, salt, corrosion, and other impurities, contamination, and degradation by a liquid-impermeable protection film or layer, such as a passivation layer, formed over the chip side of the substrate.

Over the chip side is disposed a non-conductive sealing layer 3614, optionally with a sanitary fabric or rubbery exterior surface as is familiar in bandages. The bottom side of the substrate, shown here facing down, may be coated with an adhesive layer (e.g., polyacrylate, methacrylate, acrylamide, or a silastic gel layer). The adhesive layer may be 1 to 2 mils thick and is of a kind approved for skin contact.

A release backing (not shown) may also be applied over the adhesive layer for protecting the adhesive from contaminants and for removal just prior to adhering the sensor patch 3700 to an surface in need of a temperature measurement, for example a forehead or arm of a human subject.

Furthermore, in one embodiment the sensor patch 3700 can be disposable and, as described above in conjunction with FIG. 1, no onboard power source may be required because the sensor patch may be configured to be powered entirely by an NFC emission from a smart device, such as a smart phone by holding the NFC antenna of the smart device in radio proximity to the antenna coil 3704 (passive NFC mode) and in other embodiments battery power may be used (active power mode). In some embodiments, the device can switch between passive and active modes.

Still referring to FIG. 34, alternate embodiments of the device 3700 are contemplated. For example, the circuit board 3702 and components may be assembled into a housing having similar but slightly expanded dimensions and an internal cavity in which the circuitry fits. In use, the device in a sealed housing is compatible with internal surfaces of the mouth, for example, such that the temperature sensor 3710 is inserted under a tongue, for example, and the coil is allowed to rest outside the lip. A smart device, which acts as a data reader and an energy source for the device, is brought near the coil and the measurement is performed under control of instructions executed by the smart device logic circuitry. In this way, a battery-less device is realized that is either disposable or can be washed and reused.

Alternatively, the housing may contain a battery in operative linkage to the NFC circuit and microprocessor. In one embodiment, the battery is rechargeable. A battery is not shown in FIG. 37, but the circuit can be modified to include one inside the antenna coil or on the underside of the circuit board as for example described for device 3400 (FIG. 26A). In other combinations, for example, features of embodiments described in conjunction with FIGS. 1-33B and 35-37 may be applicable to the sensor patch 3700 of FIG. 34.

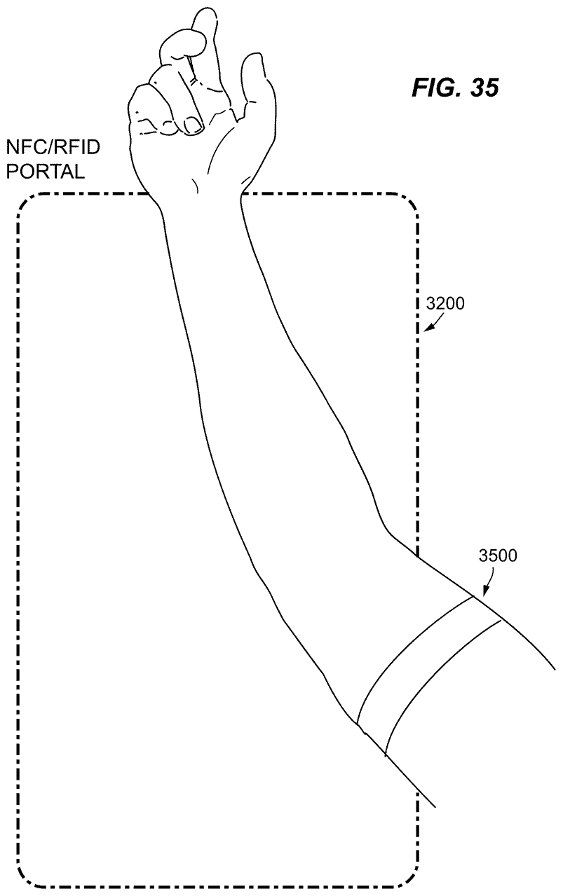

FIG. 35 shows a temperature sensor patch 3500 with a band-like form factor for use with an NFC portal 3200. The band can be mounted so that the thermistor is in intimate contact with the skin proximate to the axilla so as to provide a close approximation of core body temperature.

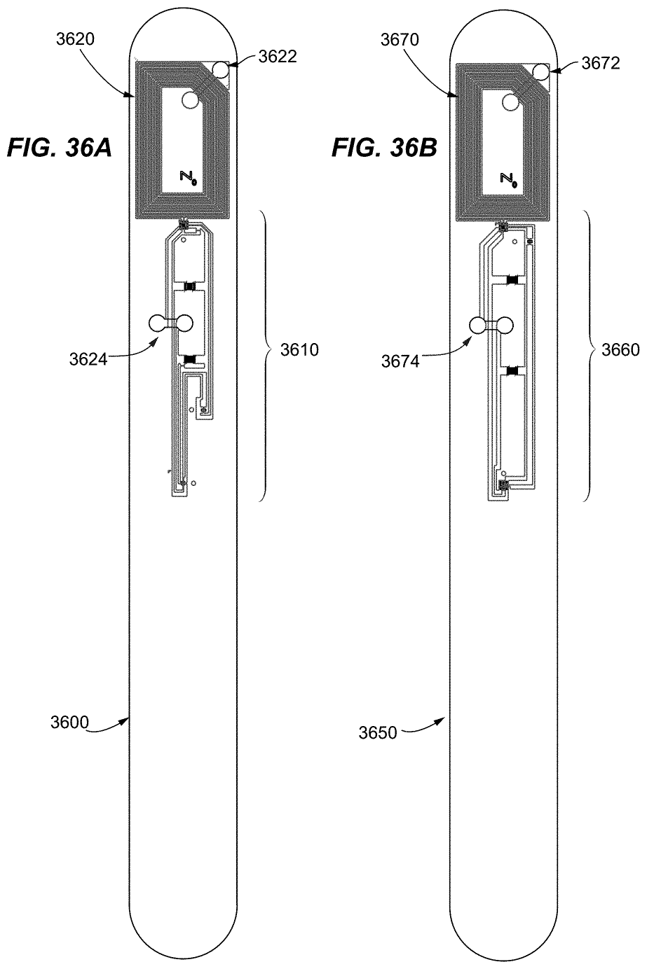

FIGS. 36A and 36B are views of two alternative band structures 3600, 3650, each with an NFC/RFID antenna and components useful as a temperature monitor. The band 3600, 3650 is wrapped around an arm below the shoulder so that body temperature in the axilla can be measured. Temperature in the armpit more closely resembles core body temperature. Each band includes a temperature sensor circuit 3610,3660 that is passively powered by an NFC/RFID antenna 1220,1270. Battery-powered devices of this kind may also include Bluetooth radiosets if desired. The antenna is built into the band structure. Printed features 3622 and 3672 are coil bridge connections. Features 3624 and 3674 are reference resistors printed in place. Other printed features may include capacitors, transistors, and batteries. Wrist bands may be configured to operate similarly. Bands that are not reporting a valid body temperature will cause an alert. These bands may also include a Bluetooth radioset and antenna and may include battery power as described in other embodiments.

FIG. 37 is a view of an exemplary circuit 3900 operating as an NFC/RFID and Bluetooth sensor data logger in cooperation with a smart phone 440. The circuit includes proprietary encryption and security features for protecting data.

The patch 3900 is a flexible, breathable pad that adheres to the skin of a subject in need of clinical monitoring and can include multiple sensors. The patch as supplied is in a dormant state so that power is not wasted during storage. To initiate a measurement, the user must first wake up the patch. In this example, a smartphone 440 is used to scan a QR Code on the back of the patch (not shown) to start the procedure. Voice instructions are recited to the user and the system can respond to questions from the user. On first use, the user will be prompted to install an Application onto the user's smartphone that controls the patch sensor. This process is initiated from an IP Address that links to a system server component and uses the smartphone as a client system component. It is the smartphone that interfaces with the patch. The first instruction is to boot the patch out of its dormant state and to initiate battery power to the MCU 3902 via SPMU 3904. This also initiates clock functions in the patch. The clock has a real time clock component for keeping time and a countdown component for setting interrupts used to wake up the patch at programmable time intervals, such as for making autonomous temperature measurements without the need for the user to be in attendance. The logic circuit may include a clock with many days of memory for intermittent data logging, and the clock is configured to be operated in a low-power oscillator mode in which it counts down to a next wake up for periodic activation of the sensor devices. The logic circuit may contain memory for logging temperatures as a function of time, for uploading data to higher computer systems, and may include local notifier capabilities such as an LED, a display, or a speaker. The logic circuit also may include a time-out timer, and after measuring a temperature and reporting any data in memory, will time out by moving to a low-power mode. The logic circuit also may include interrupts for wake up due to sensor activity or radio activity. During periods of standby, battery drainage can be limited to self-discharge rate or 0.65 uA, whichever is greater. The device also may include a communications circuit that is operable in fully passive conditions (i.e, even if the battery is fully discharged) for reporting a temperature readout or a full memory download via NFC/RFID modulation so that some functionality is recoverable.

MCU 3902 core includes a BTLE radioset 3910. Because the BTLE radioset is also awakened when battery power is established, the user can move the smartphone 440 away from the patch but continue to receive and exchange data and instructions with it. After this, the NFC/RFID radio is needed only if the battery is dead and a near-field link via NFC antenna Q1 is needed only to passively operate the patch so as to recover data in the event of a power failure.

Because the device is battery powered and wearable, it can automate temperature logging during the night and will relay temperature data to the companion smartphone via BT radio. The smartphone can in turn relay the data to the cloud host, and the cloud host can analyze a series of temperatures to either adjust the frequency of temperature monitoring by sending commands back to the smartphone, or can send a notification such as an alarm to wake up and tend to the patient if a fever is spiking. The cloud host can also monitor battery use and generate an alarm if the battery is failing. It can collect data to relieve the device from the need for a larger data storage capacity. In this way, a small disposable device can serve as a nurse over a convalescence of several days. Packages of the disposable device may be supplied. Data can be transferred from an old patch to a new patch by keeping the user profile open. Between transmissions, data is stored in flash memory 3940.

In use, a sick child in one bedroom can be monitored by a user in another bedroom. The cloud host 1000 will also receive data forwarded from the smartphone and can intervene if a worrisome trend in the temperature data emerges. The server system has the capacity to awaken a parent, for example, and to offer access to medical advice, or to simply serve as a reminder to check the child's condition. Other remote alerts, such as a movement alert, can be enabled by providing additional sensor types in the patch and means to transmit sensor output to the smartphone.

The system can also cause the patch to return to a dormant state. A zero-power passive memory of RFID-accessible memory 3907 can be used to store a history of use so that the user can be reminded of a last set of measurements in the flash memory of the device and asked whether to keep them or discard them, for example.

The RFID memory 3907 also offers an elegant method for encryption. By including a private key in the patch RFID memory 3907, that key is sent on an internal bus to the Bluetooth radio unit 3910 and serves as the encryption key. The system learns the encryption key from the tag TID during the initial NFC/RFID link, so that it is not broadcast at any time in data sharing. The manufacturer retains a table of TID numbers and their corresponding encryption keys. It is the RFID private key that aids in achieving HIPPA compliance of the Bluetooth radio devices. The private key is transmitted over the nearfield radio link so that it cannot be intercepted by remote devices, and by sharing the key with the smartphone, Bluetooth data encryption can be carried out locally without sharing keys with the server system component 1000.

The smartphone is tasked to determine a location of the patch during the initial pairing and on any subsequent data exchange with the server system component 1000, will be asked to transmit the most recent location. As described in Example II, the location data can be used for clinical triage, fever mapping, for quickly summoning assistance, and for cross-checking records to ensure data fidelity with the appropriate user profile, and also to comply with local jurisdictional issues and directives of public health authorities.

The NFC/RFID radioset 3906 can be a MAX66242 (MAXIM Integrated, San Jose Calif.) operable with 4096 Kbit EEPROM memory and an I2C master/slave interface and integrated NFC/RFID ISO 15693 interface. The MAX66242 includes an encryption key in memory and can be programmed to authenticate with a data logger during an initialization and then share encrypted data only with readers having the required key. Because smart devices function not only as NFC/RFID readers but also as Bluetooth and WiFi radio transceivers, the authentication performed initially by NFC/RFID can be used to secure and encrypt transmissions made across the LAN radio bands as well. In one instance, a private key is communicated on the nearfield communications link and is used subsequently on the LAN Bluetooth communications link. The private key may be 128 bit encryption for medical quality data protection, for example, and can be stored in MCU 3902 once it is transmitted to the device from the smartphone 440. Any application installed on the smartphone can also be made HIPPA compliant so as to secure the system uplink to higher level secure servers. By virtue of the inherent intimacy of the NFC/RFID radio link, which operates only with the smart device 440 within inches of the coil, the security of the more readily accessible Bluetooth transmissions can also be encrypted in subsequent data and command radio exchanges.

In another embodiment, power management can include a MOSFET switch in the SPMU 1904 for example that turns on and off V_{cc} power to a sensor platform 3920 in response to an encrypted signal from a transceiver. The setup of the device requires that an authorized smartphone 440 or other smart device be placed close to the sensor patch and that an NFC/RFID authentication and initialization protocol be executed. Generally this is achieved by installing an “app” in the smart device that recognizes patches having the required NFC/RFID radioset, sending an authentication signal to the patch so as to initialize the patch for use, causing the patch to enter the battery-powered “data logging” mode with intermittent power to the sensor package, and then receiving data over the LAN radio. If the battery power fails or is somehow disconnected, onboard memory stack can still be retrieved using an NFC/RFID reader such as a smartphone 440.

A digital data logger of this kind can be used in a variety of packages for measuring a clinical condition of a patient, and may have device/apparatus form factors of a patch, a strip, a spot, or a thermometer package as shown in any of the preceding figures. These can be household thermometers 2100 of the kind illustrated in use in FIG. 27A or thermometer sensor patches 3300 illustrated in FIG. 33A for example, while not limited thereto.

Alternatively an SOC approach can be taken in which an entire power, sensor and comms system with NFC/RFID and LAN radiosets is incorporated in a single chip, and the board includes the NFC/RFID coil Q1 and the Bluetooth antenna 3930 in a compact footprint.

The device 3900 may also include provision for location and tracking services. The requirements for location services can include a GPS unit 3940, or can include a variety of more energy efficient location trackers that are described in U.S. patent application Ser. No. 17/163,403, titled, “XCB TRACKING DEVICES, METHODS AND SYSTEMS”, which is incorporated herein in full by reference. Location services can be provided in any of the devices illustrated here using the referenced methods and components if the energy budget of the device is sufficient to support them. Location services are complementary to data logging services and both geostamped and timestamped records may be stored in memory for periodic upload to a network host 1000.

In other combinations, for example, features of embodiments described in conjunction with FIGS. 1-36B and 35-37 may be applicable to the sensor patch 3700 of FIG. 34.

### Example I

An NFC connection is made between a sensor patch and a smart device. The sensor patch is a dual-radio dual-power device mounted in a bandage that can be applied to the skin. Energization and temperature measurement occur when the two devices are brought to less than about 1 cm of one another, although actual distances will depend upon a variety of factors. In a simple example, a bandage with internal sensor patch adherent to a wound will broadcast a first temperature during the initial stage of the healing process but may broadcast a higher temperature if inflammation indicative of infection develops. The broadcast is made over a BT channel and can be uplinked to a cloud host using a smartphone so that the cloud host can archive the data and make notifications if needed to a physician or other caregiver.

### Example II

The devices and systems disclosed here offer significant advances in telemedicine that may reduce costs of medical care while improving outcomes and providing safer environments and working conditions. When the device 3900 is combined with the network system 2100, the capacity to continuously monitor patient health is combined with the capacity locate the patient, as will be described in this example.

In a busy emergency room, there is a need to triage patient condition and schedule resources. Less seriously ill patients will have to wait until more critical emergency cases are stabilized or transported to other facilities. However, a patient's condition may deteriorate unexpectedly, for example a GI bleeder complaining of heartburn may experience a sudden drop in hematocrit. Or a COVID patient with a mild cough may undergo a sudden pneumatic embolism that was not present during initial examination. These conditions can be detected using sensor patches, such as patches configured to measure oxyhemoglobin by blood oximetry. Other sensors may measure pulse rate and cardiac ejection fraction as an index of heart function. A sensor patch 3404 configured with a dual radio can report the data to a hub 3606 that forwards the data to a local or cloud server for interpretation and archiving. Any change in baseline vital signs or sensor signal data can result in a notification. By providing staff with portable smart devices (or even an “app” installed on a smartphone given to each member of the ER team), the notifications can be quickly assessed and acted on.

As practiced, the sensor patches are mounted on each new patient as an initial step in reception and the devices automatically are registered with a hub that begins tracking the patient and receiving sensor data. If all is well, the patient is asked to take a seat and wait for examination.

However, in the event of an emergency, the inventive devices and systems also provide a second key piece of information—where is the distressed patient? Is the distressed patient waiting in a hallway, collapsed in a bathroom, or still being processed in the waiting room. The devices report patient location, gaining valuable time to rush aid to the patient. Although location can be sometimes deduced from patient identification, the time spent looking up this information is time lost. Instead the device allows the physician and nursing staff to “see” the patient location in the virtual radio space created by the hub. The hub can also know the staff locations, and can guide staff to the patient by mapping out the destination and the shortest path to get there.

Location can be determined by radio proximity, based on a hub measurement of signal intensity, or can be coordinate based, such as by GPS, AGPS, POLTE, triangulation as recited in our earlier filings (U.S. patent application Ser. Nos. 17/163,403, 16/575,315, 15/863,731), all of which are incorporated in full by reference. Location can also be determined using a smartphone instead of a hub, or using a smartphone in combination with a hub.

The sensor data is an important supplement to clinical evaluation in triaging patients. Virtual monitoring data, sent by the sensor patches, helps identify patients having critical conditions.

In one example, by using a sensor array, the sensor patch 3900 can be placed on top of an artery, such as on the wrist, without exactly knowing where the radial artery is—the sensor patch 3900 can be configured to detect the strongest signal and assess pulse rate accordingly. And by comparing the pulse wave deformation along a series of sensors that follow the artery, the sensor patch 3900 can integrate the size of the pulse wave. The integration, when placed in the context of a database of measurements made of patients in critical condition, can be used to alert the user or a caregiver or administrator that something is amiss, that some new event is occurring, or that a series of measurements over time shows a worsening condition. When confined with electrophysiology of the heart by use of an electrocardiogram (EKG), measurement of peripheral pulse volume can provide a convincing indication of cardiac ejection volume, a major predictor for morbidity and mortality in congestive heart failure.

Thus, use of the sensor patch 3900 in combination with a smart device, when sensor data is transmitted to an experienced clinician or to a cloud facility for making computerized evaluations, can result in improved outcomes by assisting in emergency room triage and speeding interventions in real time.

### Example III

The sensor patch 3900 in combination with a smart device may also be used to improve homecare. Real time data, transmitted to an experienced clinician or to a cloud facility for making computerized evaluations, can result in improved outcomes by getting people to the emergency room when needed and by giving them the peace of mind to stay home when no intervention is called for. The data can be encrypted to meet medical grade encryption standards.

### INCORPORATION BY REFERENCE

All of the U.S. patents, U.S. patent application publications, U.S. patent applications, foreign patents, foreign patent applications and non-patent publications referred to in this specification and related filings are incorporated herein by reference in their entirety for all purposes.

The disclosure set forth herein of certain exemplary embodiments, including all text, drawings, annotations, and graphs, is sufficient to enable one of ordinary skill in the art to practice the teachings disclosed herein. Various alternatives, modifications and equivalents are possible, as will readily occur to those skilled in the art in practice of the teachings of the disclosure. The examples and embodiments described herein are not limited to particularly exemplified materials, methods, or structures and various changes may be made in the size, shape, type, materials, steps, number and arrangement of parts described herein. For example, one, or a device, may omit one or more steps of a disclosed embodiment of a method, or may add one or more steps to a disclosed embodiment of a method. All embodiments, alternatives, modifications and equivalents may be combined to provide further embodiments of the present disclosure without departing from the true spirit and scope of the disclosed subject matter.

In general, in the following claims, the terms used in the written description should not be construed to limit the claims to specific embodiments described herein for illustration, but should be construed to include all possible embodiments, both specific and generic, along with the full scope of equivalents to which such claims are entitled. Accordingly, the claims are not limited in haec verba by the disclosure.

We claim:

1. A method for patient triage using a dual-radio/dual-power radiotag with sensor package applied to a patient, which comprises:
(a) attaching the radiotag to the patient, the radiotag having a tracking signal;
(b) associating the radiotag with a network,
(c) reading sensor data from the radiotag; and,
(d) dispatching a caregiver to the location of the tracking signal in response to sensor data indicative of a clinical condition that requires an intervention.

2. A radiotag for triage and patient tracking, which comprises an attachable package having a processor, a sensor circuit with temperature sensor connected to the processor, a switching regulator connected to supply V_{cc} to the processor, an NFC/RFID radio and antenna configured to supply a first input voltage to the switching regulator when powered by an external NFC/RFID field, a battery configured to supply a second input voltage to the switching regulator when powered by the battery, a Bluetooth radio and antenna connected to the processor, wherein the NFC/RFID radio and antenna are configured for receiving commands from and reporting clinical data to an external NFC/RFID radiotag reader when powered by the external NFC/RFID field; and, wherein the Bluetooth radio and antenna are configured for receiving commands from and reporting clinical data to a Bluetooth radiotag transceiver when powered by the battery.

3-10. (canceled)

11. A system for wireless patient triage, which comprises:
(a) a sensor patch formed of a flexible substrate having an adhesive side and a device side, the adhesive side for adherence to the skin of a patient, the device side including a processor, an NFC radio and antenna, a Bluetooth radio and antenna, a battery, a switching regulator configured to supply power to the device from the battery or from an external NFC radio field if the battery is inactive or depleted, and a sensor or sensors configured to measure a clinical condition or conditions when adhered to a patient, wherein the sensor patch is configured:
(i) to transmit sensor data indicative of the clinical condition or conditions on the NFC radio if the switching regulator selects the NFC radio as a power source;
(ii) to transmit the sensor data and a tracking signal on the Bluetooth radio if the switching regulator selects the battery as a power source;
(b) a local area network receiver (“hub”) configured to receive a Bluetooth radio transmission that includes the sensor data and the tracking signal from the sensor patch and to output a location data of the device in real time from the tracking signal; and,
(c) an administrative host configured to monitor the location and the sensor data, to dispatch a caregiver to the location in response to sensor data indicative of a clinical condition that requires an intervention, and to maintain an administrative database for logging patient data, sensor data, and location data.

12. The system of claim 11, further comprising a smart device with NFC radio and Bluetooth radio, wherein the smart device is configured to initialize the sensor patch over an NFC radio field between the NFC radio of the sensor patch and the NFC radio of the smart device.

13. The system of claim 12, wherein the smart device is configured to bootstrap a Bluetooth radio link between the Bluetooth radio of the sensor patch and the Bluetooth radio of the smart device.

14. The system of claim 12, wherein the smart device is configured to associate a unique digital identifier of the sensor patch with a patient record in the administrative database.

15. The system of claim 12, wherein the smart device is a smartphone that includes a software package that enables secure local radio communication with the sensor patch.

16. The system of claim 12, wherein the smart device is configured to read a unique digital identifier from a QR code or an RFID memory cache of the sensor patch.

17. The system of claim 16, wherein the smart device is configured to read a private key from the RFID memory cache, and the private key for HIPPA-compliant encrypted radio communications.

18. The system of claim 13, wherein the smart device is configured to bootstrap a radio data link between the sensor patch and the hub.

19. The system of claim 11, wherein the hub is configured to report sensor data and location data for storage in the administrative database of the administrative host.

20. The system of claim 11, wherein the hub is configured to relay commands to the smart device from the administrative host.

21. The system of claim 11, wherein the administrative host is configured to associate the sensor data and the location data with a patient record in the administrative database, and to dispatch a caregiver to the current location of the sensor patch if the clinical condition requires an intervention.

22. The system of claim 12, wherein, in response to sensor data indicative of a clinical condition that requires an intervention, the smart device is configured to receive a command from the administrative host, the command dispatching a caregiver to the location of the sensor patch.

23. The system of claim 12, wherein the smart device is configured to triangulate a location of the sensor patch from radio signals received by the smart device and the hub in combination.

24. The system of claim 11, wherein the sensor patch is adherable to a patient's skin over an underlying peripheral artery.

25. The system of claim 24, wherein the sensor patch includes a force sensor array.

26. The system of claim 25, wherein the force sensor array is configured to output sensor data that defines a spatial map of deformations in an underlying peripheral artery.

27. The system of claim 22, wherein the sensor patch includes a hematological sensor.

28. The system of claim 27, wherein the hematological sensor is a blood pulse oximeter,

29. The system of claim 28, wherein the blood pulse oximeter is configured to output sensor data that includes a pulse waveform and a blood oxygenation index.

30. The system of claim 22, wherein the sensor patch includes a temperature sensor.

31. The system of claim 21, wherein the administrative host is configured to track the location of caregivers and to send a map to the smart device when dispatching a caregiver to a first location in response to sensor data indicative of a clinical condition that requires an intervention.

32. The system of claim 31, wherein the map includes a display showing a marked path to the first location.
